(19) United States
(12) Patent Application Publication
(10) Pub. No.: US 2004/0072325 A1
(43) Pub. Date: Apr. 15, 2004

(54) Transformation system of fungus belonging to the genus monascus

(76) Inventors: Hideharu Anazawa, Tokyo (JP); Yoko Kato, Tokyo (JP); Tadashi Nagashima, Aichi (JP); Kenzo Miyoshi, Aichi (JP); Takao Yamazumi, Aichi (JP)

Correspondence Address: FOLEY AND LARDNER, SUITE 500, 3000 K STREET NW, WASHINGTON, DC, 20007, US

(21) Appl. No.: 10/312,503

(22) PCT Filed: Jun. 28, 2001
(86) PCT No.: PCT/JP01/05596

(30) Foreign Application Priority Data
Jun. 28, 2000 (JP) 2000-195142

(51) Int. Cl. C12N 1/20

(52) U.S. Cl. 435/252.3

(57) ABSTRACT

Provided are a novel DNA which encodes a selection marker appropriate for the transformation system of filamentous fungi of the genus Monascus and for expression of a recombinant DNA, and a novel DNA which functions as a promoter and a terminator. There is also provided a novel transformation system of filamentous fungi of the genus Monascus, in which filamentous fungi of the genus Monascus is transformed with a vector comprising the DNA encoding the above selection marker and the DNA encoding a desired protein in addition to promoter and terminator sequences to obtain a transformant; culturing the transformant; and isolating at high levels the expression product of the recombinant DNA.

### TECHNICAL FIELD

The present invention relates to a reproducible transformation system in filamentous fungi belonging to the genus Monascus of the class Hemiascomycetes. More specifically, the present invention relates to transformation of filamentous fungi of the genus Monascus with recombinant DNA, and expression of recombinant DNA by the resultant, novel transformant. In addition, the present invention relates to a novel DNA sequence which can be used as a selection marker for transformation, and novel DNA sequences which can be used as a promoter and a terminator for expressing recombinant DNA.

As DNA which encodes the novel selection marker disclosed in the present invention, nitrate reductase gene and acetamidase gene which are derived from Monascus purpureus are exemplified. In addition, as the novel DNA sequences which can be used as a promoter and as a terminator for expressing recombinant DNA, glyceraldehyde-3-phosphate dehydrogenase gene, alcohol dehydrogenase gene and acid phosphatase gene which are derived from Monascus purpureus are exemplified.

### BACKGROUND ART

Development of genetic recombination techniques has realized mass production of useful protein by microorganisms. A convenient host for this purpose is a prokaryotic system, such as Escherichia coli or Bacillus subtilis. In particular, Escherichia coli is the most frequently used as a host. However, the Eschericia coli system cannot fully satisfy various needs, because most proteins produced by E. coli are insoluble, and because the E. coli system undergoes such constraints that no sugar chain can be added to the proteins when they are secreted, and the like.

In contrast, production of useful proteins in a eukaryotic microorganism system, such as yeast and filamentous fungi, is useful for the following reasons. Yeast of the genus Saccharomyces and filamentous fungi, such as Aspergillus, have been utilized for a long time for alcoholic beverages production and fermented food production. In addition to use as very safe hosts, they have been commercially used as enzyme-producing and secreting fungi. Therefore, their ability to produce and secrete a useful protein in a soluble form is high. Further, sugar chains can be added to the protein when it is produced by secretion, because they are eukaryotic organisms.

Among filamentous fungi, Aspergillus nidulans has been researched best and genetic findings of Aspergillus nidulans have been accumulated. Specifically, various selection markers and regulator genes for transformation have been reported for Aspergillus nidulans, and a transformation system using Aspergillus nidulans host has been developed. Selection marker genes that have been used for transformation of Aspergillus nidulans are orotidine-5′-phosphate decarboxylase gene Pyr4 [Biochem. Biophys. Res. Commun., 112, 284-289 (1983)] of Neurosora crassa, acetamidase gene amdS [Gene, 26, 205-221 (1983)], and ornithine carbamyl transferase gene argB [Enzyme Microb. Technol., 6, 386-389 (1984)] of Aspergillus nidulans and the like.

In addition, transformation of Aspergillus niger [EMBO J., 4, 475-479 (1985)] using amdS gene of Aspergillus nidulans, and transformation of Aspergillus oryzae [J. Ferment. Bioeng., 74, 389-391 (1992)] using amdS gene of Aspergillus oryzae have been reported. Further, transformation of Aspergillus niger [Gene, 37, 207-214 (1985)] and transformation of Aspergillus oryzae [Agric. Biol. Chem., 51, 2549-2555 (1987)] using argB gene of Aspergillus nidulans have been reported. Furthermore, transformation of Aspergillus niger using nitrate reductase gene niaD of Aspergillus niger [Gene, 78, 157-166 (1989)], and transformation of Aspergillus oryzae [Mol. Gen. Genet., 218, 99-104 (1989)] using niaD gene of Aspergillus oryzae have been reported. Moreover, transformation of Aspergillus niger [Mol. Gen. Genet., 206, 71-75 (1987), Curr. Genet., 11, 499-503 (1987)] using orotidine-5′-phosphate decarboxylase gene pyrG of Aspergillus niger has been reported.

On the other hand, the filamentous fungi belonging to the genus Monascus taxonomically belong to the family Hemiascomycetes. About 20 species or 70 different strains of the filamentous fungi belonging to the genus Monascus have been isolated and identified to date.

Filamentous fungi belonging to the genus Monascus have been used from ancient times in China, Taiwan and the like, mainly as koji for brewing and as fungi for producing a colorant or a flavoring agent. Specifically, they are used in red liquor, Chinese wine, red Chinese style cheese, pickles of flesh and of vegetables, and sauteed food. In addition, filamentous fungi contribute to the gastronomic culture of Okinawa in Japan, which is represented by tofu carbuncle, steamed rice with red beans, red rice-cake sweets and the like.

The filamentous fungus belonging to the genus Monascus produces a significant amount of red pigment, and its koji presents dark red so it is generally called red koji mold. The red pigments produced by red koji have been used as a colorant preferentially, because a red food material is scarce and the pigments are safe as they are natural pigments. Red pigments consisting of rubropunctatin, monascorubrin and the like are industrially produced as natural coloring agents which are extracted and isolated by organic solvents. Since synthetic red pigments can no longer be used because of concerns over their possible carcinogenesis, consumption of the natural red pigments is increasing. Red pigments produced by red koji have been reported to have an antiseptic effect and anti-cancerous effect in addition to their application as a coloring agent. Therefore, improvement of their productivity and their application as a pharmaceutical preparation are expected.

Further, red koji has an alcohol production ability higher than that of other koji and has been also used as a flavoring agent which adds a sweet aroma. Further, using its high alcohol production ability, application of red koji to alcohol fermentation using biomass has been attempted. In contrast to the limited assimilability of yeast, Aspergillus can be expected to have assimilability for a wider variety of substances. Thus, it is considered that Aspergillus has a high utility value. Actually, production of red alcoholic beverages has been attempted by combining red pigment production with alcohol fermentation, of red koji.

Furthermore, red koji is known to have a variety of functionalities that are not seen in other koji, such that it produces physiologically active substances comprising manifold metabolites, for example, various organic acids and peptides, in addition to enzymes, such as protease and amylase. A variety of industrial applications of red koji have been attempted.

Red koji is the sole type of koji used also as a Chinese herbal medicine. Specifically, red koji has been used widely and regularly since ancient times as a Chinese herbal medicine which helps digestion and improves blood circulation. Monacolin K having a strong hypotensive effect and inhibitory effect on cholesterol biosynthesis has been reported as a physiologically active substance produced by red koji [J. Antibiot., 32, 852-854, (1979), J. Antibiot., 33, 334-336, (1980), Japanese Patent Examined Publication No. 60-44914]. In addition to Monacolin K, the presence of a substance having a hypotensive effect has been reported [Food and Development, 28, 47-50 (1993)]. Furthermore, red koji has also been reported to produce a substance, such as Monascidin A, which has antibacterial activity against those of the genera Bacillus, Streptococcus and Pseudomonas [Fermentation and Industry, 43, 544-552 (1985)].

As described above, similarly to yellow koji mold, Aspergillus oryzae, filamentous fungus belonging to the genus Monascus (red koji mold) is recognized as a very safe filamentous fungus that has long been consumed as food, and as a fungus producing industrially and pharmaceutically useful substances [Fermentation and Industry, 43, 544-552 (1985); Science and Technology for Miso, 45, 322-328 (1997)].

The above knowledge also suggests that establishing both a transformation system of filamentous fungi belonging to the genus Monascus and a recombinant expression system using filamentous fungi of the genus Monascus is industrially very useful.

However, there is no known transformation system of the filamentous fungi belonging to the genus Monascus.

### DISCLOSURE OF THE INVENTION

An object of the present invention is to establish a transformation system of the filamentous fungus belonging to the genus Monascus, red koji mold, which has long been consumed as food similarly to yellow koji mold and which is thus very safe for the human, and provide a method for producing a protein using transformants established by the transformation system.

We have thoroughly studied the host-vector system of filamentous fungi belonging to the genus Monascus. Thus, we have completed the present invention by establishing the transformation system of this strain.

The present invention encompasses the following inventions.

(1) In a method for transforming filamentous fungi, the improvement comprising using a filamentous fungus belonging to the genus Monascus as a host.

(2) The method according to (1), wherein the filamentous fungus belonging to the genus Monascus is Monascus purpureus.

(3) The method according to (1) or (2), which comprises introducing into a host a recombinant DNA that is obtainable by incorporating into one vector a DNA encoding a marker for selecting a transformant and a DNA encoding a desired protein.

(4) The method according to (1) or (2), which comprises introducing into a host two types of recombinant DNAs, one of which is obtainable by incorporating a DNA encoding a marker for selecting a transformant into a vector, and the other of which is obtained by incorporating a DNA encoding a desired protein into a vector.

(5) The method according to (3) or (4), wherein the DNA encoding a marker for selecting a transformant is selected from the group consisting of DNA encoding the nitrate reductase of filamentous fungi, DNA encoding acetamidase of filamentous fungi, DNA encoding ornithine carbamyl transferase of filamentous fungi and DNA encoding orotidine -5′-phosphate decarboxylase of filamentous fungi.

(6) The method according to (3) or (4), wherein the DNA encoding a marker for selecting a transformant is a DNA comprising a nucleotide sequence represented by any one of SEQ ID NOS: 1,2,3,5 and 7.

(7) The method according to (3) or (4), wherein the DNA encoding a marker for selecting a transformant is a DNA hybridizing to the DNA comprising the nucleotide sequence represented by SEQ ID NO: 1 or 3 under stringent conditions, and encoding a protein having activity substantially equivalent to nitrate reductase; or a DNA hybridizing to the DNA comprising the nucleotide sequence represented by SEQ ID NO: 5 or 7 under stringent conditions, and encoding a protein having activity substantially equivalent to acetamidase.

(8) The method according to (3) or (4), wherein the recombinant DNA has a promoter which is located upstream of the DNA encoding a desired protein and is derived from a gene selected from the group consisting of an alcohol dehydrogenase gene, an acid phosphatase gene, a glyceraldehyde-3-phosphate dehydrogenase gene, a phosphoglycerate kinase gene, a glucoamylase gene, a phytase gene, a protease gene and a cellulase gene.

(9) The method according to (3) or (4), wherein the recombinant DNA has a terminator which is located downstream of the DNA encoding a desired protein and is derived from a gene selected from the group consisting of an alcohol dehydrogenase gene, an acid phosphatase gene, a glyceraldehyde-3-phosphate dehydrogenase gene, a phosphoglycerate kinase gene, a glucoamylase gene, a phytase gene, a protease gene and a cellulase gene.

(10) The method according to (8) or (9), wherein the alcohol dehydrogenase gene, the acid phosphatase gene or the glyceraldehyde-3-phosphate dehydrogenase gene is derived from the filamentous fungi belonging to the genus Monascus.

(11) The method according to (10), wherein the alcohol dehydrogenase gene comprises the nucleotide sequence represented by SEQ ID NO: 9, the acid phosphatase gene comprises the nucleotide sequence represented by SEQ ID NO: 13, and the glyceraldehyde-3-phosphate dehydrogenase gene comprises the nucleotide sequence represented by SEQ ID NO: 17 or 18.

(12) The method according to (8), wherein the promoter is capable of enhancing gene expression in the presence of lower alcohol.

(13) The method according to (12), wherein the lower alcohol is ethanol or methanol.

(14) The method according to (8), wherein the promoter comprises a DNA having a nucleotide sequence of 50 or more consecutive nucleotides between positions 1 and 615 of the nucleotide sequence of SEQ ID NO: 9.

(15) The method according to (8), wherein the promoter comprises a DNA having a nucleotide sequence of 50 or more consecutive nucleotides between positions 1 and 1013 of the nucleotide sequence of SEQ ID NO: 13.

(16) The method according to (8), wherein the promoter comprises a DNA having a nucleotide sequence of 50 or more consecutive nucleotides in the nucleotide sequence of SEQ ID NO: 17.

(17) The method according to (8), wherein the promoter comprises a DNA having a nucleotide sequence of 50 or more consecutive nucleotides between positions 1 and 1078 of the nucleotide sequence of SEQ ID NO: 18.

(18) The method according to (9), wherein the terminator comprises a DNA having a nucleotide sequence of 50 or more consecutive nucleotides between positions 1950 and 4142 of the nucleotide sequence of SEQ ID NO: 9..

(19) The method according to (9), wherein the terminator comprises a DNA having a nucleotide sequence of 50 or more consecutive nucleotides between positions 2633 and 3831 of the nucleotide sequence of SEQ ID NO: 13.

(20) The method according to (9), wherein the terminator comprises a DNA having a nucleotide sequence of 50 or more consecutive nucleotides between positions 2347 and 2456 of the nucleotide sequence of SEQ ID NO: 18.

(21) The method according to (3) or (4), wherein the DNA encoding a desired protein comprises the nucleotide sequence represented by any one of SEQ ID NOS: 1, 3, 5, 7, 9, 11, 13 and 15.

(22) The method according to (3) or (4), wherein the desired protein is selected from the group consisting of nitrate reductase, acetamidase, alcohol dehydrogenase II and acid phosphatase that are derived from filamentous fungi belonging to the genus Monascus, and phytase that is derived from Aspergillus niger.

(23) The method according to (3) or (4), wherein the desired protein comprises an amino acid sequence represented by any one of SEQ ID NOS: 4, 8, 12 and 16.

(24) The method according to (3) or (4), wherein the DNA encoding a desired protein is a DNA encoding a protein comprising a desired protein and a signal peptide of the secretory protein of a filamentous fungus which peptide has been added to the N-terminus of the desired protein.

(25) The method according to (24), wherein the signal peptide of the secretory protein of the filamentous fungus is a signal peptide of phytase of Aspergillus niger, acid phosphatase of Monascus purpureus, or Taka-amylase A of Aspergillus oryzae.

(26) A transformant of a filamentous fungus belonging to the genus Monascus, which is obtainable by any one of the methods according to (1) to (25).

(27) A method of producing a protein, which comprises culturing the transformant according to (26),until a desired protein is produced and accumulated in a culture, and recovering the protein therefrom.

(28) A DNA, which comprises a nucleotide sequence of 50 or more consecutive nucleotides between positions 1 to 615 of the nucleotide sequence of SEQ ID NO: 9.

(29) A DNA, which comprises a nucleotide sequence of 50 or more consecutive nucleotides between positions 1950 and 4142 of the nucleotide sequence of SEQ ID NO: 9.

(30) The DNA according to (28), which is capable of enhancing gene expression in the presence of lower alcohol.

(31) The DNA according to (30), wherein the lower alcohol is ethanol or methanol.

(32) A DNA, which comprises a nucleotide sequence of 50 or more consecutive nucleotides between positions 1 and 1013 of the nucleotide sequence of SEQ ID NO: 13.

(33) A DNA, which comprises a nucleotide sequence of 50 or more consecutive nucleotides between positions 2633 and 3831 of the nucleotide sequence of SEQ ID NO: 13.

(34) A DNA, which comprises the nucleotide sequence of SEQ ID NO: 17.

(35) A recombinant DNA, which comprises as a selection marker a DNA comprising the nucleotide sequence represented by SEQ ID NO: 1 or 3, or a DNA which hybridizes under stringent conditions to a DNA comprising the nucleotide sequence represented by SEQ ID NO: 1 or 3 and encodes a protein having activity substantially equivalent to that of nitrate reductase.

(36) A recombinant DNA, which comprises as a selection marker a DNA comprising the nucleotide sequence represented by SEQ ID NO: 5 or 7, or a DNA which hybridizes under stringent conditions to a DNA comprising the nucleotide sequence represented by SEQ ID NO: 5 or 7 and encodes a protein having activity substantially equivalent to that of acetamidase.

(37) A recombinant DNA, which comprises as a promoter a DNA according to (28), (32) or (34).

(38) A recombinant DNA, which comprises as a terminator a DNA according to (29) or (33).

(39) A protein, which comprises an amino acid sequence represented by any one of SEQ ID NOS: 4, 8, 12 and 16.

(40) A protein, which comprises an amino acid sequence wherein one or more amino acid residues are deleted, substituted and/or added in the amino acid sequence represented by any one of SEQ ID NOS: 4, 8, 12 and 16, and has activity equivalent to that of the protein comprising the amino acid sequence represented by any one of SEQ ID NOS: 4, 8, 12 and 16.

(41) A DNA, which encodes the protein according to (39) or (40).

(42) The DNA according to (41), which comprises a nucleotide sequence represented by any one of SEQ ID NOS: 1, 3, 5, 7, 9, 11, 13 and 15.

(43) A DNA, which hybridizes to the DNA according to (42) under stringent conditions, and encodes a protein having activity substantially equivalent to that of a protein comprising an amino acid sequence represented by any one of SEQ ID NOS: 4, 8, 12 and 16.

(44) An oligonucleotide, which comprises a nucleotide sequence that is identical to that of 15 to 60 consecutive nucleotides in a nucleotide sequence represented by any one of SEQ ID NOS: 1, 3, 5, 7, 9, 11, 13 and 15, or comprises a nucleotide sequence that is complementary to that of the oligonucleotide.

The present specification includes the contents as disclosed in the specification and/or the drawings of Japanese Patent Application No. 2000-195142, which is the priority document of the present application.

The present invention is hereinafter explained in detail.

1. Transformation Method Using a Filamentous Fungus Belonging to the Genus Monascus as a Host

The transformation method of the present invention is characterized in that filamentous fungus belonging to the genus Monascus is used as a host.

The method of transforming filamentous fungi belonging to the genus Monascus has been completed by applying a transformation method for filamentous fungi belonging to the genus Aspergillus using the standard known host-vector system [Biochem. Biophys. Res. Commun., 112, 284-289 (1983), Gene, 26, 205-221 (1983), Enzyme Microb. Technol., 6, 386-389 (1984), EMBO J., 4, 475-479 (1985), J. Ferment. Bioeng., 74, 389-391 (1992), Gene, 37, 207-214 (1985), Agric. Biol. Chem., 51, 2549-2555 (1987), Gene, 78, 157-166 (1989), Mol. Gen. Genet., 218, 99-104 (1989), Mol. Gen. Genet., 206, 71-75 (1987), Curr. Genet., 11, 499-503 (1987)].

Key techniques in the transformation method using a host-vector system of filamentous fungi belonging to the genus Monascus are hereinafter described in detail. The technique for which explanation is omitted can be implemented according to the above known methods. In addition, methods that can be used as basic genetic engineering methods are described in Molecular Cloning, A Laboratory Manual, Second Edition, Cold Spring Harbor Laboratory (1989) (hereinafter referred to as “Molecular Cloning 2nd Edition”), Current Protocols in Molecular Biology, John Wiley & Sons (1987-1997) (hereinafter referred to as “Current Protocols in Molecular Biology”), DNA Cloning 1: Core Techniques, A Practical Approach, Second Edition, Oxford University Press (1995), and the like. Further, genetic engineering methods that can be used herein and are specifically used for filamentous fungi are described in Applied Molecular Genetics of Filamentous Fungi [Applied Molecular Genetics of Filamentous Fungi, R. Kinghorn and G. Turner ed., p1-p27, Blckie Academic & Professional (1992)] and the like. The term “gene of protein X” described below in the specification includes a region encoding the protein X in genomic DNA, an untranslated region when transcribed to mRNA, an intron, and a regulatory region, such as a promoter, a terminator or an enhancer, which is normally present adjacent to upstream or downstream of the region encoding protein X in the genomic DNA, and which regulates expression of the region.

(1) Host

Filamentous fungi belonging to the genus Monascus used as hosts include all filamentous fungi identified as those belonging to the genus Monascus. Specific examples of such filamentous fungi include Monascus purpureus, Monascus pilosus, and Monascus ruber. Monasucus purpureus includes those strains previously known as Monascus anka, Monascus major, Monascus albidus, Monascus araneosus, and Monascus rubiginosus. In particular, Monascus purpureus is the most often used in industrial production, and is highly safe.

(2) Vector

As a vector, any known vector that can be used in the host-vector system of filamentous fungi can be used. Examples of the vector include pUC18 [Gene, 33, 103-119 (1985), TAKARA SHUZO CO., LTD.], pUC118 [Methods Enzymol. 153, 3-11 (1987), TAKARA SHUZO CO., LTD.], pBluescript II SK (+) and pBluescript II SK (−) [for both, Nucleic Acids Res., 17, 9494 (1989), STRATAGENE], and pBluescript SK (+) (STRATAGENE).

(3) A Selection Marker for Selecting Transformants

A preferred vector comprises DNA which encodes a selection marker for selecting a transformant. Since filamentous fungi belonging to the genus Monascus are highly resistant to known drugs, examples of a selection marker that can be preferably used herein include proteins other than those encoded by known drug-resistant genes, such as enzymes that are involved in assimilability of a carbon source or a nitrogen source, and are of metabolic system for various compounds acting as a carbon source or a nitrogen source; and enzymes that are involved in the requirement of nucleic acids and amino acids, and are of biosynthetic system of nucleic acids and amino acids.

Examples of a preferred selection marker include nitrate reductase, acetamidase, ornithine carbamyl transferase, and orotidine -5′- phosphate decarboxylase. Examples of DNAs encoding these selection markers include a nitrate reductase gene niaD, an acetamidase gene amdS, an ornithine carbamyl transferase gene argB, and an orotidine-5′-phosphate decarboxylase gene pyrG or pyr4. cDNAs derived from these genes can also be mentioned as examples of the DNAs encoding these selection markers.

The DNA encoding the above selection marker may be derived from either a host or an organism other than the host, so far as it can function as a selection marker in a host. Preferably, a gene derived from a host is used, which increases the chance of homologous incorporation of the introduced vector into the chromosome of the cell. Thus, reliable expression of a marker can be expected.

A gene as the selection marker derived from a filamentous fungus which is used as a host and belongs to the genus Monascus can be obtained from chromosomal DNA library by constructing a chromosomal DNA library of the filamentous fungus used as a host and performing plaque hybridization by use of genes derived from other filamentous fungi and having known nucleotide sequences as probes. Chromosomal DNA libraries of filamentous fungi belonging to the genus Monascus can be prepared as follows. First, chromosomal DNA of a filamentous fungus belonging to the genus Monascus is isolated, the DNA is cleaved into 5 to 20 kb in length using an appropriate restriction enzyme that enables insertion into the cloning site of a vector, such as EcoR I or BamH I, and then the vector is inserted into an arm cleaved at the cloning site. As for the chromosomal DNA, the cells of the filamentous fungus of the genus Monascus are frozen by liquid nitrogen, homogenized rapidly with a pestle, mixed with a TE buffer solution [10 mmol/l Tris-HCl (pH 8.0), 1 mmol/l EDTA] for suspension, mixed with an equivalent amount of lytic solution [2% SDS, 0.1 mol/l NaCl, 10 mmol/l EDTA, 50 mmol/l Tris-HCl (pH 7.0)], and then kept warm at 37° C. for 30 min for lysis. The resulting lysate is centrifuged (12,000×G) to collect a supernatant. Purification can be performed by subjecting the supernatant to, in sequence, phenol treatment, ethanol precipitation, RNase treatment, phenol treatment (twice), chloroform treatment and then ethanol precipitation. As a vector, lambda EMBL 3, lambda EMBL 4 [for both, J. Mol. Biol., 170, 827-842 (1983), STRATAGENE] and lambda DASH II (STRATAGENE) are used. After insertion of a chromosomal DNA fragment into a vector, in vitro packaging is performed using a kit, such as Gigapack Gold or Gigapack Gold III (both manufactured by STRATAGENE). Then, the packaging solution is infected with an Escherichia coli host which is appropriate for each vector. For example, when a vector is lambda EMBL 3, lambda EMBL 4 or lambda DASH II, the solution is infected with E. coli P 2392, E. coli XL1-Blue MRA or E. coli XL1-Blue MRA (P 2) (all manufactured by STRATAGENE) to amplify the λ phage in the library, thereby preparing chromosomal DNA libraries. According to the instructions attached to the products of STRATAGENE, insertion of chromosomes into vectors, in vitro packaging, and infection and proliferation of lambda phages can be performed. Fragments of 0.5 kb or more of the genomic DNA or cDNA of the gene of other filamentous fungi are labeled with horseradish peroxidase or radioactive isotope ³²P and then used as probes. These fragments can be isolated by digestion with restriction enzymes from the genomic DNA clones or cDNA clones of the gene, or can be isolated after amplification by PCR using primers designed based on the nucleotide sequence information of the gene, and using chromosomal DNA of the filamentous fungus as a template. When horseradish peroxidase-labeled probes are used, labeling of the probes, hybridization, and detection of hybridized spots can be performed according to ECL Direct Nucleic Acid Labeling and Detection System (Amersham Pharmachia Biotech) and its instructions. Labeling with ³²P, hybridization and detection of hybridized spots can be performed according to Molecular Cloning 2nd Edition.

The nitrate reductase gene derived from a filamentous fungus belonging to the genus Monascus can be obtained from a chromosomal DNA library of the filamentous fungus of the genus Monascus, using niaD gene of Aspergillus oryzae [Biosci. Biotech. Biochem., 59, 1795-1797 (1995): GenBank Accession No. D49701] as a probe. A specific example of the thus obtained nitrate reductase gene derived from a filamentous fungus belonging to the genus Monascus, is a nitrate reductase gene derived from Monascus purpureus having the nucleotide sequence of SEQ ID NO: 1. In addition, DNA having the nucleotide sequence of SEQ ID NO: 3 also encodes nitrate reductase of Monascus purpureus, and thus can be used as a DNA encoding a selection marker. Further, niaD gene of Aspergillus oryzae itself functions as a nitrate reductase gene in filamentous fungi belonging to the genus Monascus, and thus can be used as a DNA encoding a selection marker.

The acetamidase gene derived from a filamentous fungus belonging to the genus Monascus can be obtained from the chromosomal DNA library of the filamentous fungus belonging to the genus Monascus using amdS gene of Aspergillus oryzae [Gene, 108, 91-98 (1991): GenBank Accession No. D10492] as a probe, or using amdS gene of Aspergillus nidulans [Gene, 26, 205-221 (1983): GenBank Accession No. M16371] as a probe. A specific example of the thus obtained acetamidase gene is acetamidase gene derived from Monascus purpureus having the nucleotide sequence of SEQ ID NO: 5. In addition, DNA having a nucleotide sequence represented by SEQ ID NO: 7 also encodes the acetamidase of Monascus purpureus, and thus can be used as a DNA encoding a selection marker.

The amdS genes derived from Aspergillus nidulans and Aspergillus oryzae cannot be directly used as DNAs encoding selection markers, because the genes cannot function in transformants of filamentous fungi belonging to the genus Monascus. To use the amdS gene as a selection marker gene, the gene is modified to have a sequence appropriate for filamentous fungi of the genus Monascus based on the nucleotide sequence information represented by SEQ ID NO: 5 or 7.

The ornithine carbamyl transferase gene derived from filamentous fungi belonging to the genus Monascus can be obtained from the filamentous fungi belonging to the genus Monascus using argB gene [Enzyme Microb. Technol., 6, 386-389 (1984)] of Aspergillus nidulans as a probe.

The orotidine-5′-phosphate decarboxylase gene derived from a filamentous fungus belonging to the genus Monascus can be obtained from a chromosomal DNA library of the filamentous fungus belonging to the genus Monascus using pyrG gene [Curr. Genet., 16, 159-163 (1989)] of Aspergillus niger or pyr4 gene [Biochem. Biophys. Res. Commun., 112, 284-289 (1983)] of Neurospora crassa as a probe.

In addition, DNA which hybridizes to the whole or a part of the obtained DNA encoding a selection marker under stringent conditions can also be used as a DNA encoding a selection marker.

The DNA which hybridizes under stringent conditions means a DNA which can be obtained by colony hybridization, plaque hybridization, Southern blot hybridization or the like using as a probe the above-obtained DNA encoding a marker. Specifically, such a DNA can be identified by performing hybridization in the presence of 0.7 to 1.0 mol/l sodium chloride at 65° C. using a filter having DNA derived from a colony or a plaque immobilized thereto; and washing the filter at a temperature condition of 65° C. using SSC solution at 0.1 to 2 fold concentration (SSC solution at 1 fold concentration comprises 150 mmol/l sodium chloride and 15 mmol/l sodium citrate). Hybridization can be performed according to the methods described in Molecular Cloning 2nd Edition, Current Protocols in Molecular Biology, DNA Cloning 1: Core Techniques, A Practical Approach, Second Edition, Oxford University (1995), and the like. Hereinafter, the stringent conditions in the present specification denote the above conditions.

The DNA capable of hybridizing has, for example, at least 60% or more homology, preferably 80% or more homology, and further preferably 95% or more homology with the nucleotide sequence of the above-obtained DNA encoding the selection marker. A numerical value of homology described in the present specification may be the value calculated using a homology search program known by a person skilled in the art, such as BLAST [J. Mol. Biol., 215, 403-410 (1990)] or FASTA [Methods. Enzymol., 183, 63-98 (1990)], unless specifically stated. A preferred numerical value is calculated by BLAST using the default parameter (initial setting) or by FASTA using the default parameter (initial setting).

Whether the DNA capable of hybridizing under the above stringent conditions encodes a protein having activity substantially equivalent to that of nitrate reductase can be confirmed by introducing the DNA to express into a host of a mutant strain that is unable to use nitrate as nitrogen source (as described in (4)). The introduction confers assimilability of nitrate to the host, and the host becomes capable of assimilating nitrate. Thus, it is confirmed that the DNA encodes the protein when the host can grow in a minimal medium containing nitrate as a sole nitrogen source. Alternatively, it is confirmed that the DNA encodes the protein by using the DNA according to the methods described in 2 or 3 to allow the protein encoded by the DNA to be expressed in hosts filamentous fungi belonging to the genus Monascus or other organisms, or using in vitro translation; and measuring the activity of the protein (nitrate reductase) according to the method described in literature [Biochim. Biophys. Acta, 113, 51-56 (1966)].

Whether the DNA capable of hybridizing under the above stringent conditions encodes a protein having activity substantially equivalent to that of acetamidase can be confirmed by introducing the DNA into a host for expression. Since the capability of the host to assimilate acetamide is improved after introduction, it is confirmed that the DNA encodes the protein when the host can grow well in a minimal medium containing acetamide as a sole nitrogen or carbon source. Alternatively, it is confirmed that the DNA encodes the protein by using the DNA according to the methods described in 2 or 3 to allow the protein encoded by the DNA to be expressed in hosts filamentous fungi belonging to the genus Monascus or other organisms, or using in vitro translation; and measuring the activity of the protein (acetamidase) according to the method described in literature [J. Bacteriol., 111, 717-722 (1972)].

(4) Host for Selecting Transformants

To screen for transformants, DNA encoding a selection marker is preferably introduced into a host which lacks the marker function or has low marker function. Examples of the host include filamentous fungi belonging to the genus Monascus having properties equivalent to those of hosts appropriate for each selection marker used in the known methods of transformation in the filamentous fungi belonging to the above genus Aspergillus.

When nitrate reductase is used as a selection marker, a preferred host is of a mutant strain which is unable to use nitrate as a nitrogen source because of mutation in nitrate reductase gene. Introduction of the nitrate reductase gene into the mutant strain can give assimilability of nitrate, so that transformants can be selected.

Mutant strains which are unable to use nitrate as a nitrogen source because of mutation in nitrate reductase gene can be obtained by inoculating and culturing the conidia of a filamentous fungus belonging to the genus Monasucus in a minimal medium containing a mutagen, obtaining strains which grow after inoculation, and examining these strains for the assimilability of a nitrogen source. Mutagens may be any of chemical mutagens, radioactive isotopes and the like known by a person skilled in the art, and are not specifically limited. Preferably, a chemical mutagen, more preferably, chlorate is used. The term “minimal medium” means a medium comprising minimum components essential for the growth of cells of the wild type when microorganims are cultured. For example, a minimal medium for culturing filamentous fungi comprises a carbon source, a nitrogen source and inorganic salts. Such a minimal medium containing a mutagen can be appropriately prepared by a person skilled in the art. An example of a minimal medium is a plate medium (pH 5.5) consisting of 3% sucrose, 10 mmol/l glutamic acid, 0.2% KH₂PO₄, 0.05% MgSO₄. 7H₂O, 0.05% KCl and 470 mmol/l KClO₃. As filamentous fungi belonging to the genus Monascus, the desired species which should be used as a host can be employed. The temperature for culturing may be any temperature at which filamentous fungi to be used herein can grow, and is not specifically limited. A preferred temperature ranges from 15 to 40° C., and more preferably, is about 30° C. The time for culturing is not specifically limited because it can be appropriately set by a person skilled in the art, and is preferably from 15 to 20 days.

Whether strains having grown as described above can assimilate a nitrogen source can be examined by culturing these strains on minimal media containing various nitrogen sources and determining whether or not they grow on each medium. Nitrate, such as NaNO₃, is used as a nitrogen source in testing areas, and nitrite, ammonium salt, various amino acids and the like are used as nitrogen sources in control areas. An example of a minimal medium containing nitrate as a nitrogen source (testing area) is a basal plate medium (1% glucose, 0.1% KH₂PO₄, 0.05% MgSO₄-7H₂O, 0.05% KCl, 1.5% agar, pH 5.5) supplemented with 10 mmol/l NaNO₃; an example of a control area is a basal plate medium supplemented with 10 mmol/l NaNO₂, (NH₄)₂SO₄, proline, glutamic acid, alanine and the like. The temperature for culturing may be a temperature at which filamentous fungi to be used herein can grow, and is not specifically limited. A preferred temperature ranges from 15 to 40° C., and more preferably, is about 30° C. The time for culturing is not specifically limited because it can be appropriately set by a person skilled in the art, and is preferably about 3 days.

Nitrate reductase has activity to reduce nitrate to nitrite. Therefore, strains which do not grow in a minimal medium (testing area) containing nitrate as a nitrogen source, but grow in a minimal medium (control area) containing sources other than nitrate, such as nitrite, as a nitrogen source are selected as a mutant strain which is unable to use nitrate as a nitrogen source because of mutation in nitrate reductase gene.

When acetamidase is used as a selection marker, a wild type strain can be used as a host without obtaining the above mutant strain, because the filamentous fungi belonging to the genus Monascus has low assimilability of acetamide. Transformants can be selected using, as a marker, the host's enhanced assimilability of acetamide resulting from introduction of the acetamidase gene.

When ornithine carbamyl transferase is used as a selection marker, it is preferred to use as a host a mutant strain having ornithine carbamyl transferase gene showing arginine requirement. The strain grows to require no arginine by introducing ornithine carbamyl transferase gene into the mutant strain. Transformants can be selected in such a change from arginine requirement to arginine non-requirement as a marker. A mutant strain having ornithine carbamyl transferase gene can be obtained in the same manner as that for a mutant strain of Aspergillus niger having arginine requirement [Gene, 37, 207-214 (1985)]. Specifically, the conidia irradiated with ultraviolet rays to have gene mutation are cultured in a minimal medium containing no arginine (the medium comprises, for example, 1% glucose, 10 mmol/l urea, 0.1% KH₂PO₄, 0.05% MgSO₄.7H₂O, 0.05% KCl, pH 5.5). Conidia that grow in the medium are removed by means of glass filter filtration. Then, conidia that do not grow are inoculated over a minimal medium supplemented with arginine, so that strains that grow are obtained. By examining whether the obtained strains require arginine and intermediates for arginine biosynthesis system, mutant strains having mutations of ornithine carbamyl transferase gene can be obtained. Specifically, each strain is cultured on a minimal medium (for example, the medium consists of 1% glucose, 10 mmol/l urea, 0.1% KH₂PO₄, 0.05% MgSO₄.7H₂O, 0.05% KCl, 1.5% agar, pH 5.5) and on minimal media supplemented respectively with ornithine, citrulline and arginine. Finally, the strains which do not grow in a minimal medium and in a minimal medium supplemented with ornithine, but grow in a medium supplemented with citrulline, and in a minimal medium supplemented with arginine are selected as mutant strains having mutations of ornithine carbamyl transferase gene.

When orotidine-5′-phosphate decarboxylase is used as a selection marker, it is preferred to use as a host a mutant strain showing uridine requirement. The strain becomes to require no uridine by introducing the orotidine-5′-phosphate decarboxylase gene into the mutant strain showing uridine requirement. Transformants can be selected in such a change from uridine requirement to uridine non-requirement as a marker. A mutant strain showing uridine requirement can be obtained as a strain resistant against 5-fluoro orotic acid in the presence of uridine, in the same manner as employed to obtain a mutant strain of Aspergillus niger showing uridine requirement [Mol. Gen. Genet., 206, 71-75 (1987)]. Specifically, the conidia irradiated with ultraviolet rays to have mutation are cultured in a minimal medium (for example, the minimal medium consists of 1% glucose, 10 mmol/l urea, 0.1% KH₂PO₄, 0.05% MgSO₄.7H₂O, 0.05% KCl, 1.5% agar, pH 5.5) containing 10 mmol/L uridine and 1 mg/ml 5-fluoro orotic acid. Strains that grow are obtained. By examining whether the obtained strains require uridine, mutant strains showing uridine requirement can be obtained. When cultured in a minimal medium and in a minimal medium supplemented with uridine, strains which cannot grow in a minimal medium, but can grow in the minimal medium supplemented with uridine, are selected as mutant strains showing uridine requirement.

A mutant strain can also be obtained by disrupting a target gene of each of the above selection marker genes in the same manner as the method [Gene, 108, 91-98 (1991)] which involves disrupting a target gene of amdS gene of Aspergillus oryzae and the mutant strain is used as a host. Specifically, a selection marker gene is obtained from a filamentous fungus belonging to the genus Monascus used as a host by the method described in (3). Using restriction enzyme sites within a region encoding the selection marker of the gene, a DNA of several 100 bp to several kb is inserted or deleted, thereby disrupting the selection marker gene to cause it to lose its function. Then, the gene is inserted into an appropriate plasmid vector for use in transformation described in (2). Next, a filamentous fungus belonging to the genus Monascus for use as a host is transformed using the disrupted selection marker gene by the method described in (6), thereby causing homologous recombination by which a wild type selection marker gene is substituted with the disrupted selection marker gene. After transformation, the protoplast is cultured in a medium in which the gene-disrupted strain can grow, according to each type of disrupted target gene. For example, in the case of nitrate reductase gene and acetamidase, the protoplast is cultured on a minimal medium consisting of 1% glucose, 10 mmol/l urea, 0.1% KH₂PO₄, 0.05% MgSO₄.7H₂O, 0.05% KCl, 1.5% agar, and pH 5.5; in the case of omithine carbamyl transferase gene, the protoplast is cultured on a minimal medium supplemented with arginine; and in the case of orotidine-5′-phosphate decarboxylase gene, the protoplast is cultured on a minimal medium supplemented with uridine. Strains that grow are cultured on selection media appropriate for each type of disrupted target gene. That is, in the case of nitrate reductase gene, the strain is cultured in a minimal medium having nitrate as a nitrogen source; in the case of acetamidase, the strain is cultured on a minimal medium having acetamidase as a nitrogen source; in the case of ornithine carbamyl transferase gene, the strain is cultured in a minimal medium which lacks arginine; and in the case of orotidine-5′-phosphate decarboxylase gene, the strain is cultured in a minimal medium which lacks uridine. The strain which cannot survive is selected as a strain containing its disrupted (target) selection marker gene for use as a host. When a wild type selection marker gene is substituted with a disrupted selection marker gene by homologous recombination, the restriction enzyme map of a genome gene in the region comprising the selection marker gene becomes different from that of a wild type strain. Therefore, chromosomal DNA is prepared from the selected strain, and Southern blot hybridization is performed using the selection marker gene as a probe, so that disruption of the target gene can be confirmed.

(5) Expression of DNA Encoding Selection Marker

DNA encoding a selection marker obtained in (3) is subcloned to an appropriate vector of (2), and then introduced into a host for expression of the selection marker. To express the selection marker in the host, a promoter is required to be present upstream of the DNA encoding the selection marker. As the promoter, the selection marker gene's own promoter can be used. Further, substitution of the promoter with a stronger promoter enables the selection marker to function more effectively. A terminator is not always required to be present downstream of the DNA encoding the selection marker. However, the expression efficiency of the selection marker can be enhanced by locating a terminator downstream of the DNA.

Examples of a strong promoter include those derived from genes of filamentous fungi belonging to the genus Aspergillus which are known to have a strong promoter, such as alcohol dehydrogenase gene alc (including alcA, alcB, alcC), acid phosphatase aph, glyceraldehyde-3-phosphate dehydrogenase gene gpd, phosphoglycerate kinase gene pgk, glucoamylase gene glaA, phytase gene phy, protease gene pep, and cellulase gene cel.

Furthermore, a preferred promoter is derived from a filamentous fungus belonging to the genus Monascus which has been obtained based on such known promoter information. Examples of such promoters include a promoter derived from a glyceraldehyde-3-phosphate dehydrogenase gene gpd1 [GenBank Accession No. Z68498] of Monascus purpureus strain IFO4478; a promoter derived from a glyceraldehyde-3-phosphate dehydrogenase gene of Monascus purpureus strain IFO30873 obtained by PCR using a primer based on the sequence of the gpd1 gene; a promoter derived from a glyceraldehyde-3-phosphate dehydrogenase gene obtained by standard methods from filamentous fungi belonging to the genus Monascus using these glyceraldehyde-3-phosphate dehydrogenase genes as probes: a promoter derived from an alcohol dehydrogenase II gene obtained by standard methods from filamentous fungi belonging to the genus Monascus using alcB gene [Curr. Genetic., 29, 122-129 (1996): GenBank Accession No. Z48000] of Aspergillus nidulans as probes; and a promoter derived from an acid phosphatase gene obtained by standard methods from filamentous fungi belonging to the genus Monascus using aph gene [Gene, 133, 55-62 (1991): GenBank Accession No. L02420] of Aspergillus niger as probes. More specific examples of a promoter include those derived from genes, such as an alcohol dehydrogenase II gene derived from Monascus purpureus having a nucleotide sequence represented by SEQ ID NO: 9; an acid phosphatase gene derived from Monascus purpureus having a nucleotide sequence represented by SEQ ID NO: 13; and a glyceraldehyde-3-phosphate dehydrogenase gene derived from Monascus purpureus having a nucleotide sequence represented by SEQ ID NO: 17 or 18. Any sequence which can substantially function as a promoter can be used. More specific examples of a promoter include DNA having a nucleotide sequence of 50 or more consecutive nucleotides between positions 1 and 615 of the nucleotide sequence of SEQ ID NO: 9; DNA having a nucleotide sequence of 50 or more consecutive nucleotides between positions 1 to 1013 of the nucleotide sequence of SEQ ID NO: 13; DNA having a nucleotide sequence of 50 or more consecutive nucleotides between positions 1 and 1181 of the nucleotide sequence of SEQ ID NO: 17; and DNA having a nucleotide sequence of 50 or more consecutive nucleotides between positions 1 and 1078 of the nucleotide sequence of SEQ ID NO: 18.

Alcohol dehydrogenase gene is a type of housekeeping gene, and is highly expressed constantly. Thus the use of the promoter of this gene enables high expression of a recombinant gene. Further, expression of alcohol dehydrogenase II gene derived from Monascus purpureus is induced by addition of ethanol into the medium. Thus, the use of the promoter of the gene enables to induce by ethanol the expression of a recombinant gene.

An upstream sequence of the chromosome of a eukaryotic organism comprises a sequence which is capable of enhancing transcription, in addition to a promoter sequence comprising a transcription initiation site. To expect high expression of a gene, a DNA sequence having activity to enhance transcription is preferably located ahead of a promoter.

Examples of a DNA sequence which is capable of enhancing promoter activity include DNAs derived from genes of filamentous fungi belonging to the genus Aspergillus that are known to have strong promoters. Specifically, such DNA may be derived from an alcohol dehydrogenase gene alc (including alcA, alcB, alcC), acid phosphatase aph, glyceraldehyde-3-phosphate dehydrogenase gene gpd, phosphoglycerate kinase gene pgk, glucoamylase gene glaA, phytase gene phy, protease gene pep, or a cellulase gene cel.

Examples of a terminator that is located downstream of DNA encoding a marker include terminators derived from genes of filamentous fungi of the genus Aspergillus that are known to have strong promoters. Specifically, such a terminator that is preferably used herein is derived from an alcohol dehydrogenase gene alc (including alcA, alcB, alcC), acid phosphatase aph, glyceraldehyde-3-phosphate dehydrogenase gene gpd, phosphoglycerate kinase gene pgk, glucoamylase gene glaA, phytase gene phy, protease gene pep, or a cellulase gene cel. Furthermore, a preferred terminator is derived from a filamentous fungus belonging to the genus Monascus which has been obtained based on such known terminator sequence information. Examples of the terminator include a terminator derived from a glyceraldehyde-3-phosphate dehydrogenase gene gpd1 of Monascus purpureus; a terminator derived from a glyceraldehyde-3-phosphate dehydrogenase gene obtained by standard methods from filamentous fungi belonging to the genus Monascus using the gene as a probe; a terminator derived from an alcohol dehydrogenase II gene obtained by standard methods from filamentous fungi belonging to the genus Monascus using alcB gene [Curr. Genetic., 29, 122-129 (1996)] of Aspergillus nidulans as a probe; and a terminator derived from an acid phosphatase gene obtained by standard methods from filamentous fungi belonging to the genus Monascus using aph gene [Gene, 133, 55-62 (1991)] of Aspergillus niger as a probe. Any sequence which can substantially function as a terminator can be used. Specific examples of the terminator include those derived from Monascus purpureus-derived genes, such as an alcohol dehydrogenase II gene having the nucleotide sequence represented by SEQ ID NO: 9; an acid phosphatase gene having the nucleotide sequence represented by SEQ ID NO: 13; or a terminator derived from a glyceraldehyde-3-phosphate dehydrogenase gene having the nucleotide sequence represented by SEQ ID NO: 18. More specifically, examples include DNA having a nucleotide sequence of 50 or more consecutive nucleotides between positions 1950 and 4142 of the nucleotide sequence of SEQ ID NO: 9; DNA having a nucleotide sequence of 50 or more consecutive nucleotides between positions 2633 and 3831 of the nucleotide sequence of SEQ ID NO: 13; and DNA having a nucleotide sequence of 50 or more consecutive nucleotides between positions 2347 and 2456 of the nucleotide sequence of SEQ ID NO: 18.

(6) Transformation Method

Transformation of filamentous fungi belonging to the genus Monascus using a vector comprising DNA encoding a selection marker can be performed by methods such as electroporation and a protoplast method, which involves introducing DNA into a cell of a filamentous fungus, and preferably, it is performed by the protoplast method. The protoplast method can be performed as follows. First, the cell walls of a filamentous fungus are lysed in an appropriate buffer by allowing an enzyme to react on the cells, preparing protoplasts. Specifically, filamentous fungi belonging to the genus Monascus are cultured statically in a nutrient medium (1% dextrin, 1% peptone, 1% yeast extract, 0.05% KH₂PO₄, 0.05% MgSO₄.7H₂O, pH 5.2) at 30° C. for 4 to 5 days. Then, the cells are collected by filtration with a glass filter. After washing the cells with distilled water, 10 ml of a protoplast preparation solution [5 mg/ml lysing enzyme, 2.5 mg/ml Sumizyme C, 0.8 mol/l NaCl, 100 mmol/l phosphate buffer, pH 6.0] containing lysing enzymes [Sigma, catalog number L1412] and Sumizyme C (SHIN NIHON CHEMICAL CO., LTD) are added to 0.5 g of the cells. The mixture is incubated at 30° C. for 3 hours while gently shaking, thereby lysing the cell walls and preparing protoplasts. The prepared protoplasts are filtered with a glass filter to remove mycelial fragments, and then washed. The washing procedure involves adding 10 ml of 0.8 mol/l NaCl solution for re-suspending the protoplasts from which the supernatants have been removed by centrifugation at 700×G; repeating twice a step of removing supernatants by centrifugation at 700×G; adding 10 ml of solution 1 [10 mmol/l CaCl₂, 0.8 mol/l NaCl, 10 mmol/I Tris-HCl buffer solution (pH 7.5)] for suspension; and centrifuging the suspension at 700×G. Next, DNA is introduced into the protoplast prepared using a buffer containing polyethylene glycol (PEG) and CaCl₂. Specifically, the above prepared and washed protoplast is suspended in three-fourth volume of solution 1 such that the protoplast density is 10⁸ cells/ml. Then, one-fourth volume of solution 2 [40% (w/v) PEG 4000, 50 mmol/l CaCl₂, 50 mmol/l Tris-HCl buffer solution (pH 7.5)] is added to the suspension, thereby preparing a protoplast solution. 20 μl of the DNA (0.5 to 0.8 μg/μl) of the vector expressing a selection marker described in (5) is added to 200 μl of the protoplast solution, and then ice-cooled for 30 min. Subsequently, 1 ml of solution 2 is added to the solution, and then the solution is allowed to stand at room temperature for 20 min. Next, 10 ml of solution 1 is added to the solution to dilute PEG concentration, the solution is centrifuged at 700×G, and then the precipitate is collected. The resulting precipitate is suspended in 200 μl of solution 1, and then the suspension is mixed with a 0.5% soft agar medium (a medium having the same composition as the following plate medium for selection, except for the concentration of agar). The mixture is inoculated on the plate medium for selection, and then cultured at 30° C. for 10 to 14 days, thereby obtaining colonies of the transformant. The plate medium for selection is a minimal plate medium on which a host cannot grow or barely grow, but on which a host can grow well when the vector DNA is introduced therein and the selection marker is expressed. Examples of such a plate medium for selection that can be used herein include a medium (1% glucose, 0.1% KH₂PO₄, 0.05% MgSO₄-7H₂O, 0.05% KCl, 1.5% agar, pH 5.5) supplemented with 10 mmol/l NaNO₃ as a nitrogen source when a selection marker is nitrate reductase; a medium (1% glucose, 0.1% KH₂PO₄, 0.05% MgSO₄.7H₂O, 0.05% KCl, 1.5% agar, pH 5.5) supplemented with 10 mmol/l acetamide as a nitrogen source when an acetamidase gene is used as a selection marker; and a medium (1% glucose, 10 mmol/l urea, 0.1% KH₂PO₄, 0.05% MgSO₄.7H₂O, 0.05% KCl, 1.5% agar, pH 5.5) which lacks uridine and arginine when ornithine carbamyl transferase or orotidine-5′-phosphate decarboxylase is used as a selection marker.

2. Method for Producing a Protein by the Method for Transforming Filamentous Fungi Belonging to the Genus Monascus

Any desired protein can be produced using filamentous fungi belonging to the genus Monascus by the transformation method of 1. above. When a desired protein is a protein derived from a filamentous fungus belonging to the genus Monascus, high expression efficiency can be obtained.

First, DNA encoding a desired protein is obtained. When the DNA cannot be easily obtained, for example, it cannot be supplied through the sample furnishment, an oligonucleotide comprising a 20 to 30 bp sequence at the 5′ end and an oligonucleotide comprising a sequence complementary to a 20 to 30 bp sequence at the 3′ end of a region encoding a desired protein are synthesized based on the nucleotide sequence information of the DNA using a DNA synthesizer. Then, PCR using as a template cDNA or genomic DNA of an organism expressing the desired protein and using both the oligonucleotides as primers is performed, so that the DNA can be amplified and then isolated. Isolation of cDNA and genomic DNA can be performed by the methods described in Molecular Cloning 2nd Edition or Applied Molecular Genetics of Filamentous Fungi, R. Kinghorn and G. Turner ed., Blckie Acadenic & Professional (1992). PCR can be performed by the methods described in PCR, A Practical Approach, Oxford University Press (1991).

To express DNA encoding a desired protein in filamentous fungi belonging to the genus Monascus, it is required to locate the promoter described in 1. (5) upstream of the DNA. This promoter is used for expressing the transformation marker in filamentous fungi belonging to the genus Monascus. When a desired protein is a protein of filamentous fungi belonging to the genus Monascus, the promoter of the gene of the protein itself can be used. However, when a desired protein is of an organism other than the filamentous fungi of the genus Monascus, a promoter derived from a gene of filamentous fungi belonging to the genus Monascus is more preferably used than the promoter of the gene of the protein itself. Moreover, examples of a strong promoter of filamentous fungi belonging to the genus Monascus that are preferably used include a promoter derived from an alcohol dehydrogenase gene, an acid phosphatase gene, or a glyceraldehyde-3-dehydrogenase gene. In addition, if necessary, expression efficiency of the protein can be enhanced by locating the terminator described in 1. (5) above, which is used for expressing a transformation marker in filamentous fungi belonging to the genus Monascus, downstream of the DNA; and locating the DNA sequence described in 1. (5) above, which is capable of enhancing promoter activity and is used for expressing a transformation marker in filamentous fungi belonging to the genus Monascus, upstream of the above promoter, respectively. Hereinafter, the DNA, and the promoter, the terminator and the DNA sequence capable of enhancing promoter activity, which are located upstream or downstream of the DNA, are together referred to as a “protein expression unit.”

A vector is constructed by inserting the above protein expression unit into an expression vector for a selection marker described in 1. (5). Filamentous fungi belonging to the genus Monascus are transformed using the vector above according to the method of 1. (6), so that filamentous fungi belonging to the genus Monascus expressing desired proteins can be prepared. Alternatively, separately from the expression vector for the selection marker described in 1. (5), a vector is constructed by inserting the above protein expression unit into a vector described in 1. (2). Filamentous fungi belonging to the genus Monascus are co-transformed with both the vector and the expression vector for the selection marker according to the method of 1. (6), so that filamentous fungi belonging to the genus Monascus expressing desired proteins can be prepared. Co-transformation is performed according to the method of 1. (6) by simultaneously adding both the vectors (10 μl each) to protoplast, instead of adding 20 μl of the selection marker-expressing vector thereto. The thus prepared transformants of the filamentous fungus belonging to the genus Monascus are inoculated on DPC medium (2% dextrin, 0.3% peptone, 0.1% corn steep liquor, 0.05% MgSO₄-7H₂O), YPD medium (2% glucose, 2% polypeptone, 1% yeast extract) and phytase production medium (3% sucrose, 10 mmol/l NaNO₃, 0.05% MgSO₄.7H₂O, 0.05% KCl, 0.1% corn steep liquor), and then statically cultured at 30° C. for 10 to 14 days. Thus, the protein is produced and accumulated in the culture comprising the cells and the media. In addition, when an alcohol dehydrogenase II gene of Monascus purpureus is used as a promoter, culturing is performed in media containing lower alcohol, preferably ethanol or methanol as a carbon source. Thus, the promoter is induced and the production amount of a desired protein can be increased. The term “lower alcohol” means alcohol having alkyl chain with carbon number of 1 to 7.

When a signal peptide is present at the N-terminus of a desired protein, the signal peptide of the protein is cleaved, the mature protein is secreted extracellularly from a host, i.e., the filamentous fungus belonging to the genus Monascus, and then the protein is accumulated in the medium. Further, when no signal peptide is present at the N-terminus of a desired protein, the protein is generally accumulated intracellularly.

In the latter case, a desired protein can be produced by extracellular secretion as follows. First, DNA encoding a desired protein in which the signal peptide of the secretory protein of a filamentous fungus has been added to N-terminus of the desired protein is obtained. An expression vector is constructed using the DNA similarly to the above method. The transformation is performed, and then transformants are cultured. As a result, added signal peptides are cleaved, and then the desired protein is secreted extracellularly. Thus the protein can be produced. It is expected that filamentous fungi belonging to the genus Monascus can perform effective secretory production of a desired protein, since they produce and secrete various enzymes. DNA encoding a desired protein in which the signal peptide of the secretory protein of a filamentous fungus has been added to N-terminus of the desired protein is obtained as follows. First, oligonucleotides A to D are synthesized by a DNA synthesizer: oligonucleotide A comprising at its 3′ end a 10 to 30 bp sequence that is identical with the sequence of the 5′ terminus of a region encoding the signal peptide of a secretory protein gene of a filamentous fungus, or the sequence located upstream of the 5′ terminus of the region; oligonucleotide B in which a 15 to 30 bp sequence at the 5′ terminus of a region encoding a desired protein has been added to the 3′ end of a sequence that is identical with a 15 to 30 bp sequence at the 3′ terminus of the region; oligonucleotide C having a sequence complementary to that of oligonucleotide B; and oligonucleotide D comprising at its 3′ end a 15 to 30 bp sequence that is complementary to that at the 3′ end of a region encoding a desired protein or that located downstream of the 3′ end. PCR is performed using as a template genomic DNA, cDNA or isolated secretory protein gene of filamentous fungi, and using as primers oligonucleotides A and C. Thus, DNA (in which DNA encoding the N-terminus of a desired protein has been added to its 3′ side) encoding a signal peptide is amplified. Subsequently, PCR is performed using DNA encoding the desired protein (obtained above) as a template, and using oligonucleotides B and D as primers. Thus, DNA (in which DNA encoding the C terminus of a signal peptide has been added to its 5′ side) encoding the desired protein is amplified. Both the amplified DNA fragments and, oligonucleotides A and D, are mixed and PCR is performed. A region at the 3′ end of a sense strand of DNA encoding the signal peptide and a region at the 5′ end of an antisense strand of DNA encoding a desired protein are complementary to each other and thus hybridize to each other. Both the regions function as primers and templates to each other, thereby amplifying DNA encoding the desired protein in which the signal peptide of the secretory protein of filamentous fungi has been added to the N-terminus of the desired protein.

Examples of a signal peptide of the secretory protein of filamentous fungi that can be used for secretory production of the above desired protein include a signal peptide of phytase of Aspergillus niger, a signal peptide of acid phosphatase of Monascus purpureus, and a signal peptide of Taka-amylase A of Aspergillus oryzae.

In addition, a desired protein can also be expressed as a fusion protein with another protein or a peptide to facilitate its detection and purification. Examples of a protein or a peptide to be fused with a desired protein include β-galactosidase, protein A, IgG binding domain of protein A, chloramphenicol acetyltransferase, poly (Arg), poly (Glu), protein G, maltose binding protein, glutathione S-transferase, poly histidine chain (His-tag), S peptide, DNA-binding protein domain, Tac antigen, thioredoxin, green fluorescent protein, FLAG peptide and an epitope of any antibody [Akio YAMAKAWA, Experimental Medicine, 13, 469-474 (1995)].

A desired protein can be isolated and purified as described below from the culture of transformants of a filamentous fungus belonging to the genus Monascus.

When the protein is secreted extracellularly from transformants, the culture of the transformants is processed by, for example, filter filtration or centrifugal separation to obtain the culture supernatant. Purification from the culture supernatant can be performed by methods normally used to isolate and purify enzymes, such as a solvent extraction method, salting-out/desalination using ammonium sulfate or the like, sedimentation using an organic solvent, diethylaminoethyl (DEAE)-sepharose (Amersham Pharmacia Biotech), anion exchange chromatography using resin, e.g., DIAION HPA-75 (Mitsubishi Chemical Corporation), cation exchange chromatography using resin, such as S-SepharoseFF (Amersham Pharmacia Biotech), a hydrophobic chromatography method using resin, such as butyl sepharose or phenyl sepharose, gel filtration using a molecular sieve, an affinity chromatography method, a chromatofocusing method, and electrophoresis, such as isoelectric focusing.

When a desired protein is accumulated in a state of being lysed within the cells of transformants, the culture is centrifuged to collect cells within the culture. Then the cells are washed, and then homogenized using an ultrasonicator, french press, Manton Gaulin homogenizer, Dynomill or the like, thereby obtaining cell-free extract. Purification and isolation can be performed in the same manner similar to that employed for the above culture supernatant from the supernatant provided by centrifugation of the cell-free extract.

In addition, when a desired protein is expressed as intracellular insoluble particles, the cells are collected and homogenized in the same manner as described above and then centrifuged, thereby collecting fractions of the precipitate containing the insoluble particles of the protein. The insoluble particles of the protein is solubilized using a protein denaturation agent. Purification and isolation can also be performed by methods similar to the above methods. In this case, when the solubilization solution contains a protein denaturation agent, the solubilization solution is diluted or dialyzed until the concentration of the agent is lowered to the extent that no protein is denatured, so as to refold the protein to have the normal three-dimensional structure before isolation and purification.

Structural analysis on the target polypeptide (or partial polypeptide) purified herein can be made according to the method normally employed in protein chemistry, for example, the method described in protein structure analysis for gene cloning (Hisashi HIRANO, published by TOKYO KAGAKU DOZIN CO., LTD., 1993).

3. Protein Derived from Filamentous Fungi Belonging to the Genus Monascus

(1) DNA Encoding Protein Derived from Filamentous Fungi Belonging to the Genus Monascus

The present invention encompasses a protein encoded by a gene derived from filamentous fungi belonging to the genus Monascus which is obtained by the methods described in 1. (3) and (5) above, and DNA encoding the protein. In addition, DNAs encoding other proteins derived from filamentous fungi belonging to the genus Monascus can also be obtained as described below.

DNA encoding a protein derived from filamentous fungi belonging to the genus Monascus can be obtained by preparing genomic DNA libraries or cDNA libraries according to the standard methods described in Molecular Cloning 2nd Edition, Current Protocols in Molecular Biology, DNA Cloning 1: Core Techniques, A Practical Approach, Second Edition, Oxford University Press (1995) and the like. Such DNA can also be obtained by preparing cDNA libraries using a commercially available kit, such as SuperScript Choice System for cDNA Synthesis (Invitrogen) or ZAP-cDNA Synthesis Kit (STRATAGENE).

Any cloning vector can be used to prepare cDNA libraries, so far as it can autonomously replicate in Escherichia coli strain K12. For example, a phage vector or a plasmid vector may be used. Specific examples include ZAPExpress (STRATAGENE), pBluescript II SK (+) [Nucleic Acids Research, 17, 9494 (1989), STRATAGENE], ? ZAPII (STRATAGENE), ?gt10, ?gt11 [both from DNA Cloning, A Practical Approach, Oxford University Press (1985)], ? ExCell (Amersham Pharmacia Biotech), and pUC18 [Gene, 33, 103 (1985)].

Any microorganism belonging to Escherichia coli can be used as an microorganism into which a vector having cDNA incorporated therein can be introduced. Specifically, Escherichia coli XL1-Blue MRF′ (STRATAGENE), Escherichia coli Y1088 [Science, 222, 778 (1983)], Escherichia coli Y1090 [Science, 222, 778 (1983)], Escherichia coli NM522 [J. Mol. Biol., 166, 1 (1983)], Escherichia coli JM105 [Gene, 38, 275 (1985)] and the like can be used.

The nucleotide sequences in the thus prepared genomic libraries or cDNA libraries are determined by a DNA sequencer. Then the sequences are compared with their corresponding gene sequences of closely related filamentous fungi, so that proteins encoded by the nucleotide sequences can be specified. Specific examples include a protein derived from a filamentous fungus belonging to the genus Monascus and a gene encoding the protein that are specified as described below.

The nitrate reductase gene and nitrate reductase of Monascus purpureus can be specified based on the nucleotide sequence of nitrate reductase gene niaD of Aspergillus oryzae (GenBank Accession No. D49701) and the amino acid sequence (SEQ ID NO: 2) of nitrate reductase encoded by the gene. As preferred examples, the nucleotide sequence on the genome of the nitrate reductase gene of Monascus purpureus is shown in SEQ ID NO: 1, the DNA sequence of the coding region is shown in SEQ ID NO: 3, and an amino acid sequence of the nitrate reductase of Monascus purpureus specified from these sequences is shown in SEQ ID NO: 4.

The acetamidase of Monascus purpureus can be specified based on the nucleotide sequence of acetamidase gene amdS of Aspergillus oryzae (GenBank Accession No. D10492) and the amino acid sequence (SEQ ID NO: 6) of acetamidase encoded by the gene. As preferred examples, the nucleotide sequence on the genome of the acetamidase gene of Monascus purpureus is shown in SEQ ID NO: 5, the DNA sequence of the coding region is shown in SEQ ID NO: 7, and an amino acid sequence of the acetamidase specified from these sequences is shown in SEQ ID NO: 8.

Alcohol dehydrogenase II of Monascus purpureus can be specified based on the nucleotide sequence of the alcohol dehydrogenase II gene alcB of Monascus nidulans (GenBank Accession No.: Z48000) and the amino acid sequence (SEQ ID NO: 10) of alcohol dehydrogenase II encoded by this gene. As preferred examples, the nucleotide sequence on the genome of the alcohol dehydrogenase II gene of Monascus purpureus is shown in SEQ ID NO: 9, the DNA sequence of the coding region is shown in SEQ ID NO: 11, and an amino acid sequence of the alcohol dehydrogenase II specified from these sequences is shown in SEQ ID NO: 12.

Acid phosphatase of Monascus purpureus can be specified based on the acid phosphatase gene aph of Aspergillus niger (GenBank Accession No. L02420) and the amino acid sequence (SEQ ID NO: 14) of the acid phosphatase encoded by the gene. As preferred examples, the nucleotide sequence on the genome of the acid phosphatase gene of Monascus purpureus is shown in SEQ ID NO: 13, the DNA sequence of the coding region is shown in SEQ ID NO: 15, and the amino acid sequence of acid phosphatase specified from these sequences is shown in SEQ ID NO: 16.

The above protein derived from a filamentous fungus belonging to the genus Monascus is the protein of the present invention, and can be efficiently expressed by the method described in 2. However, the protein of the present invention does not include a protein having an amino acid sequence which is identical to that of a known protein.

The protein of the present invention also includes a protein comprising an amino acid sequence derived from the amino acid sequence of the protein by deletion, substitution or addition of one or more amino acids, and having the same activity as that of the protein. Such protein can also be efficiently expressed by the method described in 2. The protein comprising an amino acid sequence derived from that of the above protein by deletion, substitution or addition of one or more amino acids, and having the same activity as that of the protein can be obtained by, for example, introducing a site-directed mutation into DNA encoding proteins comprising amino acid sequences represented by the above Sequence ID Numbers, according to a site-directed mutagenesis method described in, for example, Molecular Cloning 2nd Edition; Current Protocols in Molecular Biology; Nucleic Acids Research, 10, 6487 (1982); Proc. Natl. Acad. Sci. USA, 79, 6409(1982); Gene, 34, 315 (1985); Nucleic Acids Research, 13, 4431 (1985); and Proc. Natl. Acad. Sci. USA, 82, 488 (1985). The number of amino acids that is deleted, substituted or added is not specifically limited, and preferred is the number of amino acids that can be deleted, substituted or added by the known method, such as the above site-directed mutagenesis. A preferred number of amino acids to be modified herein is 1 to several dozen, more preferably 1 to 20, further preferably 1 to 10, and further more preferably 1 to 5 amino acids.

An example of a protein which is modified by the above site-directed mutagenesis, and maintains its activity after modification is a protein that has at least 60% or more, preferably 80% or more, and further preferably 95% or more homology with the amino acid sequence of the unmodified protein when calculated by BLAST using initially set parameters. Alternatively, these numerical values of homology may be calculated by FASTA using default (initially set) parameters.

Examples of DNAs encoding proteins derived from a filamentous fungus belonging to the genus Monascus are those encoding the above proteins. Specific examples of such DNAs include a DNA comprising the nucleotide sequence represented by SEQ ID NO: 1, 3, 5, 7, 9, 11, 13 or 15. A DNA which hybridizes under stringent conditions to a part of or the whole of these DNAs can also be used as DNAs encoding proteins derived from a filamentous fungus belonging to the genus Monascus. The stringent conditions are similar to those in 1 (3). The DNA of the present invention also includes these DNAs. However, the DNA of the present invention does not include those comprising a nucleotide sequence which is the same as that of a known DNA.

s(2) Application of Proteins Derived from Filamentous Fungi Belonging to the Genus Monascus

Nitrate reductase of the present invention is useful for quantitative determination of nitric acid metabolism [Agric. Biol. Chem., 47, 2427-2433 (1983)]. Acetamidase of the present invention is useful for wastewater treatment because it has a degradation activity of acrylamide [Water Res., 16, 579-591, (1982), Genetica, 90, 133-145 (1993)]. The alcohol dehydrogenase II of the present invention is useful for modification of alcohol metabolism of microorganisms or living organisms [Gene, 51, 205-216 (1987)]. The acid phosphatase of the present invention is useful for improvement of the nutritional value of feed and efficient utilization of phosphorus (reduction of phosphorus excretion) by domestic animals, because it has a degradation activity of phytic acid contained in feed [J. Sci. Food Agric., 49, 315-324 (1989)].

(3) Oligonucleotides of DNAs Encoding Proteins Derived from Filamentous Fungi Belonging to the Genus Monascus

The present invention encompasses an oligonucleotide comprising a nucleotide sequence which is complementary to a part of or the whole nucleotide sequence of DNA encoding the protein of the present invention as described in (1) above. PCR using these oligonucleotides as a sense primer and antisense primer, respectively, enables specific amplification of DNA encoding the protein of the present invention, isolation of the DNA, and detection and quantitative determination of the DNA. When the oligonucleotides are used as primers for RT-PCR which involves extracting RNA from a sample, converting the RNA into cDNA, followed by PCR, the expression amount of a gene encoding the protein can be measured. Further, an oligonucleotide comprising a nucleotide sequence which is complementary to a part of the nucleotide sequence of DNA encoding the protein of the present invention can be used as an antisense oligonucleotide for regulation, such as suppression of expression of the protein.

Examples of the oligonucleotide include DNA comprising a nucleotide sequence which is the same as a sequence of 5 to 60 nucleotides, preferably 15 to 60 nucleotides, in the nucleotide sequence of DNA encoding the protein of the present invention described in (1), or DNA comprising a nucleotide sequence which is complementary to the DNA. Specific examples of the oligonucleotide include DNA comprising a nucleotide sequence which is the same as a sequence of 5 to 60 nucleotides, preferably 15 to 60 nucleotides in the nucleotide sequence represented by SEQ ID NO: 1, 3, 5, 7, 9, 11, 13 or 15, or DNA comprising a nucleotide sequence which is complementary to that of the DNA. Preferably, the oligonucleotides used as sense primers or antisense primers are the above-mentioned oligonucleotides, since the melting temperatures (Tm) and number of nucleotides of these oligonucleotides never differ significantly. Such oligonucleotides can be prepared by a DNA synthesizer from the nucleotide sequence information of the DNA.

Furthermore, the oligonucleotide of the present invention also includes derivatives of these oligonucleotides (hereinafter referred to as “oligonucleotide derivative”). Examples of the oligonucleotide derivative include an oligonucleotide derivative in which a phosphodiester bond in the oligonucleotide is converted to a phosphorothioate bond; an oligonucleotide derivative in which a phosphodiester bond in the oligonucleotide is converted to a N3′-P5′ phosphoamidate bond; an oligonucleotide derivative in which ribose phosphodiester in the oligonucleotide is converted to a peptide nucleic acid bond; an oligonucleotide derivative in which uracil in the oligonucleotide is substituted with C-5 propynyl uracil; an oligonucleotide derivative in which uracil in the oligonucleotide is substituted with C-5 thiazole uracil; an oligonucleotide derivative in which cytosine in the oligonucleotide is substituted with C-5 propynyl cytosine; an oligonucleotide derivative in which cytosine in the oligonucleotide is substituted with phenoxazine-modified cytosine; an oligonucleotide derivative in which ribose in the oligonucleotide is substituted with 2′-O-propyl ribose; and an oligonucleotide derivative in which ribose in the oligonucleotide is substituted with 2′-methoxyethoxy ribose [Saibo Kogaku(Cell Technology), 16, 1463 (1997)].

(4) Expression of Proteins Derived from a Filamentous Fungus Belonging to the Genus Monascus

The protein as described in (3) above can be expressed at high levels in the host-vector system of filamentous fungi belonging to the genus Monascus of the present invention. Further, this protein can also be expressed using host-vector systems of organisms or cells other than those of the genus Monascus. The above protein can be obtained by allowing the DNA of the present invention to be expressed in a host cell according to the methods described in Molecular Cloning 2nd Edition or Current Protocols in Molecular Biology, for example, the method as described below, in addition to the method using the host-vector system of filamentous fungi belonging to the genus Monascus of the present invention.

DNA encoding the protein of the present invention, for example, DNA comprising a sequence of any one of SEQ ID NOS: 1, 3, 5, 7, 9, 11, 13 and 15, or a DNA fragment of appropriate length including the region encoding the protein of the present invention is prepared, and the DNA is inserted downstream of a promoter of an appropriate expression vector, thereby preparing a recombinant vector. The recombinant vector is introduced into a host cell appropriate for the expression vector. If necessary, DNA is prepared to have a nucleotide sequence (section) encoding the protein of the present invention which is modified by substituting a nucleotide(s) to have optimum codons for expression in the host cell. This DNA is useful for efficient production of the protein of the present invention. As described above, the DNA of the present invention can also be applied for the purposes other than as a selection marker, expression promoter and terminator of gene recombination in host filamentous fungi.

a) Hosts and Vectors

Any cells, such as bacteria, yeast, filamentous fungi, animal cells, insect cells and plant cells, that can express a target gene can be used as host cells.

An expression vector that is used herein is capable of autonomously replicating in the above host cells or can be incorporated into a chromosome, and has a promoter at a position which enables transcription of DNA encoding the protein of the present invention.

When prokaryotes, such as bacteria, are used as host cells, DNA comprising a sequence containing no intron, for example, DNA comprising a sequence of SEQ ID NO: 3, 7, 11 or 15 is used as a DNA encoding the protein of the present invention. A preferred recombinant vector comprising the DNA encoding the protein of the present invention is capable of autonomously replicating in prokaryotes, and comprises a promoter, ribosome binding sequence, DNA encoding the protein of the present invention and a terminator. DNA regulating a promoter may also be included. Examples of the expression vector include pKK223-2, (Amersham Pharmacia Biotech), pGEX-2T (Amersham Pharmacia Biotech), pSE420 (Invitrogen), pLEX (Invitrogen), pET-3a (Novagen), pGEMEX-1 (Promega), pQE-30 (QIAGEN), pCAL-c (STRATAGENE), pEGFP (Clontech), pKYP10 (Japanese Published Unexamined Patent Application No. 110600/83), pKYP200 [Agric. Biol. Chem., 48, 669 (1984)], pTrS20 (Japanese Published Unexamined Patent Application No. 22979/91), and pTerm2 (Japanese Published Unexamined Patent Application No. 22979/91). Any promoter which can function in a host cell may be used. Examples of such a promoter include those derived from the gene of Escherichia coli or of Escherichia coli phage, such as trp promoter (Ptrp), lac promoter, P_{L} promoter, P_{R} promoter and T7 promoter. Further, artificially designed and modified promoters, such as a promoter having two Ptrp lined in series, tac promoter, lacT7 promoter and letI promoter may also be used. Preferably, a space between a Shine-Dalgarno sequence, which is a ribosome binding sequence, and the initiation codon is 6 to 18 bp. A terminator is not always necessary, but when located downstream of the DNA encoding the protein of the present invention, the expression efficiency can be enhanced.

Examples of the host cells include microorganisms belonging to the genus Escherichia, the genus Serratia, the genus Bacillus, the genus Brevibacterium, the genus Corynebacterium, the genus Microbacterium, the genus Pseudomonas and the like, such as Escherichia coli, Serratia ficaria, Serratia fonticola, Serratia liquefaciens, Serratia marcescens, Bacillus subtilis, Bacillus amyloliquefacines, Brevibacterium ammoniagenes, Brevibacterium immariophilum, Brevibacterium saccharolyticum, Brevibacterium flavum, Brevibacterium lactofermentum, Corynebacterium glutamicum, Corynebacterium acetoacidophilum, Microbacterium ammoniaphilum, Pseudomonas putida and the like, and Escherichia coli is preferred. Examples of Escherichia coli include Escherichia coli HB101, Escherichia coli JM105, Escherichia coli BL21, Escherichia coli GI724, Escherichia coli BL21 (DE3) pLysS, Escherichia coli JM109, Escherichia coli JM109 (DE 3), Escherichia coli M15 (pREP4) and Escherichia coli 13009 (pREP4). Any method for introducing a recombinant vector can be used, so far as it is a method for introducing DNA into the above host cells. Examples of such a method include a method using calcium ion [Proc. Natl. Acad. Sci. USA, 69, 2110 (1972)], a protoplast method (Japanese Published Unexamined Patent Application No. 248394/88), and a method described in Gene, 17, 107 (1982) and Mol. Gen. Genet., 168, 111 (1979).

When filamentous fungi are used as hosts, protein can be expressed in a manner similar to that employed for expression in filamentous fungi belonging to the genus Monascus in 2. A vector used herein comprises DNA encoding the protein of the present invention inserted downstream of a promoter that functions in the filamentous fungus acting as a host. Examples of promoters include promoters derived from alcohol dehydrogenase gene, acid phosphatase gene, glyceraldehyde-3-phosphate dehydrogenase gene, phosphoglycerate kinase gene, glucoamylase gene, phytase gene, protease gene, cellulase gene and the like.

Examples of a host include Aspergillus nidulans, Aspergillus niger, Aspergillus oryzae and Trichoderma reesei. Transformation can be performed by the protoplast method [Gene, 26, 205-221 (1983)].

When yeast is used as a host cell, examples of an expression vector include YEP13 (ATCC37115), YEp24 (ATCC37051), YCp50 (ATCC37419), pHS19 and pHS15. Any promoter which can be expressed in a yeast strain may be used. Examples of such a promoter include a promoter of the gene in glycolytic pathway, such as hexose kinase; PHO5 promoter, PGK promoter, GAP promoter, ADH promoter, gal 1 promoter, gal 10 promoter, heat-shock protein promoter, MFα1 promoter and CUP 1 promoter.

Examples of host cells include microorganisms belonging to the genus Saccharomyces, the genus Schizosaccharomyces, the genus Kluyveromyces, the genus Trichosporon, the genus Schwanniomyces, the genus Pichia, the genus Candida and the like, such as Saccharomyces cerevisiae, Schizosaccharomyces pombe, Kluyveromyces lactis, Trichosporon pullulans, Schwanniomyces alluvius and Candida utilis. Any method for introducing a recombinant vector may be used, so far as it is a method for introducing DNA into yeast. Examples of such a method include electroporation [Methods Enzymol., 194, 182 (1990)], a spheroplast method [Proc. Natl. Acad. Sci. USA, 75, 1929 (1978)], a lithium acetate method [J. Bacteriol., 153, 163 (1983)] and a method described in Proc. Natl. Acad. Sci. USA, 75, 1929 (1978).

When animal cells are used as host cells, examples of an expression vector include pcDNA3.1 (+) (Invitrogen), pEFI/HisA (Invitrogen), pCMV-Script (STRATAGENE), pEGFP-C1 (Clontech) pAGE103 [J. Biochem., 101, 1307 (1987)], pAGE107 [Japanese Published Unexamined Patent Application No. 22979/91; Cytotechnology, 3, 133 (1990)], pAS3-3 (Japanese Published Unexamined Patent Application No. 227075/90), and pCDM8 [Nature, 329, 840 (1987)]. Any promoter which can function in an animal cell can be used. Examples of such a promoter include a promoter of IE (immediate early) gene of cytomegalic inclusion disease virus (CMV), SV40 early promoter, promoter of retrovirus, metallothionein promoter, heat shock promoter and SRa promoter. In addition, enhancer of IE gene of human CMV may be used together with the promoter.

Examples of a host cell include Namalwa cell which is a human cell, COS cell which is a monkey cell and CHO cell which is Chinese hamster cell. Any method for introducing a recombinant vector into an animal cell may be used, so far as it is a method for introducing DNA into an animal cell. Examples of such a method include electroporation [Cytotechnology, 3, 133 (1990)], a calcium phosphate method [Japanese Published Unexamined Patent Application No. 227075/90], and a lipofection method [Proc. Natl. Acad. Sci. USA, 84, 7413 (1987), Virology, 52, 456 (1973)].

When an insect cell is used as a host, protein can be expressed by methods described in, for example, Current Protocols in Molecular Biology; Baculovirus Expression Vectors, A Laboratory Manual, W. H. Freeman and Company, New York (1992); Bio/Technology, 6, 47 (1988); and the like. Specifically, protein can be expressed by co-introducing a recombinant gene introduction vector and Baculovirus into an insect cell so as to obtain a recombinant virus in the culture supernatant of the insect cell, and allowing the recombinant virus to infect insect cells.

Examples of a vector for gene introduction used in this method include pVL1392, pVL1393 and pBlueBac4.5 (all manufactured by Invitrogen). Examples of Baculovirus include Autographa californica nuclear polyhedrosis virus and the like, which infect insects of the family Barathra.

Examples of an insect cell that can be used herein include Sf9 and Sf21 which are cells of Spodoptera frugiperda [Baculovirus Expression Vectors, A Laboratory Manual, W. H. Freeman and Company, New York (1992)] and High 5 which is a cell of Trichoplusia ni (Invitrogen). Examples of a method for co-introducing the above recombinant gene-introduced vector and the above Baculovirus into insect cells so as to prepare a recombinant virus include a calcium phosphate method (Japanese Published Unexamined Patent Application No. 227075/90) and a lipofection method [Proc. Natl. Acad. Sci. USA, 84, 7413 (1987)].

When a plant cell is used as a host cell, examples of an expression vector include Ti plasmid and tobacco mosaic virus vector. Any promoter which can be expressed in a plant cell may be used. Examples of such a promoter include ³⁵S promoter of cauliflower mosaic virus and rice actin 1 promoter. Examples of a host cell include cells of plants, such as tobacco, potato, tomato, carrot, soybean, rape, alfalfa, rice, wheat, barley and the like. Any method for introducing a recombinant vector may be used, so far as it is a method for introducing DNA into a plant cell. Examples of such a method include a method using Agrobacterium (Japanese Published Unexamined Patent Application No. 140885/84, Japanese Published Unexamined Patent Application No. 70080/85, WO 94/00977), electroporation (Japanese Published Unexamined Patent Application No. 251887/85), and a method using particle gun (a gene gun) (Japanese Patent Nos. 2606856 and 2517813).

b) Culture of Transformants and Production of Proteins

The protein of the present invention can be produced by culturing the transformants obtained as described above in media, allowing the protein to be produced and accumulated in the culture, and recovering the protein from the culture. The transformants of the present invention can be cultured in media according to the method normally employed for culturing hosts.

When transformants are obtained using prokaryotes, such as Escherichia coli, or eukaryotic microorganisms, such as filamentous fungi, yeast and the like as hosts, a medium for culturing the transformants may be either a natural or synthetic medium, so far as it contains sources and the like assimilable by the transformants, such as carbon sources, nitrogen sources and inorganic salts, so that the transformants can be efficiently cultured in the medium. Any carbon source which is assimilable by the transformants may be used. Examples of such a carbon source that can be used herein include glucose, fructose, sucrose, molasses containing these compounds, carbohydrate, such as starch and starch hydrolysate, organic acid, such as acetic acid and propionic acid, and alcohol, such as ethanol and propanol. Examples of a nitrogen source that can be used herein include ammonium salt of inorganic acid or organic acid, such as ammonia, ammonium chloride, ammonium sulfate, ammonium acetate and ammonium phosphate, other nitrogen-containing compounds, and peptone, meat extract, yeast extract, corn steep liquor, casein hydrolysate, soybean cake and soybean cake hydrolysate, and various fermentation microbial cells and digested products thereof. Examples of the inorganic salt that can be used herein include potassium dihydrogen phosphate, dipotassium hydrogen phosphate, magnesium phosphate, magnesium sulfate, sodium chloride, ferrous sulfate, manganese sulphate, copper sulfate and calcium carbonate.

Culturing is performed under aerobic conditions, such as shaking culture or submerged culture with aeration and agitation. Culturing temperature is not specifically limited, and is preferably 15 to 40° C. Culturing time is not specifically limited, and is preferably 16 hours to 7 days. pH during culturing is preferably maintained from 3.0 to 9.0. pH can be adjusted with inorganic or organic acids, alkali solution, urea, calcium carbonate, ammonia or the like. If necessary, antibiotics, such as ampicillin or tetracycline, may be added in media while culturing. When microorganisms transformed with recombinant vectors using inductive promoters are cultured, inducers may be added to media if necessary. For example, when microorganisms transformed with recombinant vectors using lac promoters are cultured, isopropyl-β-D-thiogalactopyranoside or the like may be added to media, and when microorganisms transformed with recombinant vectors using trp promoters are cultured, indole acrylic acid or the like may be added.

Examples of media that can be used for culturing transformants obtained using animal cells as hosts include generally employed RPMI1640 medium [JAMA, 199, 519 (1967)], Eagle's minimal essential medium (MEM) [Science, 122, 501 (1952)] Dulbecco's modified Eagle's medium (MEM) [Virology, 8, 396 (1959)], 199 medium [Proceeding of the Society for the Biological Medicine, 73, 1 (1950)] and these media supplemented with fetal calf serum or the like.

Various conditions for culturing are not specifically limited. Preferably, culturing is performed under conditions of pH 6 to 8 at 30 to 40° C. in the presence of 5% CO₂ and the like for 1 to 7 days. If necessary, antibiotics such as kanamycin or penicillin may be added to media while culturing. Further, production amount can be elevated using a gene amplification system with dihydrofolate reductase gene or the like according to the method disclosed in Japanese Published Unexamined Patent Application No. 227075/90.

Examples of media for culturing transformants obtained using insect cells as hosts include generally employed TNM-FH medium [Pharmingen], Sf-900 II SFM medium (Invitrogen), ExCell400, ExCell405 [both manufactured by JRH Biosciences], and an insect medium (Grace) [Nature, 195, 788 (1962)]. Various conditions for culturing are not specifically limited. Preferably, culturing is performed under conditions of pH 6 to 7 at 25 to 30° C. and the like for 1 to 5 days. If necessary, antibiotics, such as gentamycin, may be added to media while culturing.

Transformants obtained using plant cells as hosts can be cultured as cells, or cultured after their differentiation into plant cells or organs. Examples of media that can be used for culturing the transformants include generally employed Murashige and Skoog (MS) medium, and White medium, and these media supplemented with plant hormone such as auxin, cytokinin or the like. Various conditions for culturing are not specifically limited. Preferably, culturing is performed under conditions of pH 5 to 9 at 20 to 40° C. and the like for 3 to 60 days. If necessary, antibiotics, such as kanamycin or hygromycin, may be added to media while culturing.

As described above, the protein of the present invention can be produced by culturing according to standard culturing methods transformants derived from microorganisms, animal cells or plant cells comprising recombinant vectors in which DNA encoding the protein has been incorporated; allowing the transformants to produce and accumulate the protein; and recovering the protein from the culture.

As the method for producing the protein of the present invention using a plant individual, mention may be made of a method comprising cultivating by a known method [Tissue Culture, 20 (1994), Tissue Culture, 21 (1995), Trends. Biotechnol., 15, 45 (1997)] a transgenic plant to which DNA encoding the protein has been introduced; allowing the protein to be produced and accumulated in the plant; and recovering the protein from the plant.

The protein of the present invention can be produced by a method by which the protein is produced within the host cell; a method by which the protein is secreted out of the host cell; or a method by which the protein is produced on the outer membrane of the host cell. Any of these methods can be selected, depending on alteration of the structure of host cells employed or of the protein to be produced. When the protein of the present invention is produced within a host cell or on the outer membrane of a host cell, the protein can be actively secreted out of the host cell according to the method of Paulson et al [J. Biol. Chem. 264, 17619 (1989)], the method of Lowe et al [Proc. Natl. Acad. Sci. USA, 86, 8227 (1989), Genes Develop., 4, 1288 (1990)] or methods disclosed in Japanese Published Unexamined Patent Application No. 336963/93, and WO 94/23021. In other words, by means of genetic recombination techniques, the protein of the present invention can be actively secreted out of the host cells, by allowing the protein to be expressed in such a form that a signal peptide is added to and ahead of, the polypeptide comprising the active site of the protein.

Further, the protein of the present invention can be expressed as a fusion protein with another protein or a peptide in order to facilitate the detection and purification. Examples of a protein or a peptide to be fused with the protein of the present invention include β-galactosidase, protein A, IgG binding domain of protein A, chloramphenicol acetyltransferase, poly (Arg), poly (Glu), protein G, maltose binding protein, glutathione S-transferase, polyhistidine chain (His-tag), S peptide, DNA-binding protein domain, Tac antigen, thioredoxin, green fluorescent protein, FLAG peptide and an epitope of any antibody [Akio YAMAKAWA, Experimental Medicine, 13, 469-474 (1995)].

Proteins having sugar chains added thereto can be obtained by expression of the protein of the present invention in any one of a filamentous fungus, yeast, animal cell, insect cell or plant cell.

Isolation and purification of the protein produced by the transformants of the present invention can be performed by the method described in 2.

### BRIEF DESCRIPTION OF THE DRAWINGS

FIG. 1 shows detection of pMA-niaD and pANPHY1 that have been introduced into Monascus purpureus strain SN2-30-4, by Southern blotting of chromosomal DNA. Lanes 1 to 4 denote transformants in which only pMA-niaD has been introduced, lane 5 denotes host strain SN 2-30-4, and lanes 6 to 9 denote transformants in which pMA-niaD and pANPHY1 have been co-introduced. The numbers and lines on the left denote the position of each marker and chain length (kb), respectively.

FIG. 2 shows Northern blotting detection of ADH2 mRNA of Monascus purpureus strain IFO 30873 which has been cultured in various media containing different carbon sources. Culture conditions for each lane (the carbon source in a medium, static culture or shaking culture) are shown on the right side.

FIG. 3 shows a process for constructing pMGON-HLY. HLY represents a gene comprising the signal sequence of chicken lysozyme and a human lysozyme structural gene.

FIG. 4 shows a process for constructing pMGB-TAA. TAA represents Taka-amylase A gene.

FIG. 5 shows a process for constructing pMAB-PHY.

FIG. 6 shows a process for constructing pMGB-PHY.

FIG. 7 shows a process for constructing pMAPA-PHY.

FIG. 8 shows a process for constructing phytase gene having the signal peptide of APH.

FIG. 9 shows a process for constructing phytase gene having the signal peptide of Taka-amylase A.

FIG. 10 shows a process for constructing pMGB-tPHY.

### BEST MODE FOR CARRYING OUT THE INVENTION

The present invention is more specifically described by the following examples. However, these examples are given only for illustrative purposes, and do not limit the technical scope of the present invention.

### EXAMPLE 1

### Isolation of Nitrate Reductase-Deficient Strain (niaD⁻ Strain)

Using Monascus purpureus wild strain [Institute for Fermentation, Osaka (IFO) Accession No. IFO30873, hereinafter referred to as strain IFO30873], a nitrate reductase-deficient strain was isolated by the following method.

5 mm mycelial block was picked out, of Monascus purpureus strain IFO 30873 previously cultured on slant media for conservation [Bacto potato-dextrose (Difco) 24 g/l, 1.5% agar (Difco), pH 5.5], transferred to fresh potato-dextrose agar plate media (the plate medium was prepared to have the same composition as that of the above slant medium), and then cultured at 30° C. for 7 to 10 days, so that the conidia started to grow on the media. A sterilized 5 ml of 0.01% (v/v) Tween 80 aqueous solution was added to the plate medium, and then the conidia were suspended well in the solution using a inoculating loop. The suspension was filtered with G3 glass filter to recover the conidia.

The recovered conidia were inoculated into selective plate media (3% sucrose, 10 mmol/l glutamic acid, 0.2% KH₂PO₄, 0.05% MgSO₄-7H₂O, 0.05% KCl, 470 mmol/l KClO₃, pH 5.5), and then cultured at 30° C. for 15 to 20 days. Colonies that had grown were used for the following experiments. Colonies capable of growing on the selective plate media appeared at a rate of one colony per about 10,000 conidia.

### EXAMPLE 2

### Identification of Nitrate Reductase-Deficient Strain (niaD⁻ Strain)

A deficient gene locus on the nitric acid metabolic pathway in the mutant strain provided in Example 1 above was identified by examining the presence or absence of growth on six types of plates as shown below.

(1) 10 mmol/l NaNO₃+basal plate medium (1% glucose, 0.1% KH₂PO₄, 0.05% MgSO₄.7H₂O, 0.05% KCl, 1.5% agar, pH 5.5)

(2) 10 mmol/l NaNO₂+basal plate medium

(3) 10 mmol/l (NH₄)₂SO₄+basal plate medium

(4) 10 mmol/l proline+basal plate medium

(5) 10 mmol/l glutamic acid+basal plate medium

(6) 10 mmol/l alanine+basal plate medium

The mutant strains inoculated on the above 6 types of plates were cultured at 30° C. for 3 days. As a result, the mutant strains obtained in Example 1 grew on any one of plates (2) to (6); but did not grow on plate (1), suggesting a possible deficiency in their nitrate reductase activity. One of the strains was used as a host for gene introduction and transformation as Monascus purpureus strain SN2-30-4.

### EXAMPLE 3

### Preparation of a Chromosomal Library

Similarly to Example 1, 5 mm mycelial block was scraped, of Monascus purpureus strain IFO30873 previously cultured on slant media for conservation, and then transferred into a 500 ml Erlenmeyer flask with baffle containing 100 ml of dextrin-peptone medium (2% dextrin, 1% peptone, 0.5% KH₂PO₄, 0.1% MgSO₄.7H₂O), and then subjected to static culture at 30° C. for 10 days. This culture solution was filtered with G1 glass filter, and then washed twice with sterilized water, thereby collecting approximately 0.5 g of mycelia.

The collected mycelia were placed between pieces of paper towel, compressed for dewatering, and then the mycelia were put into a mortar cooled at −80° C. After liquid nitrogen was poured into the mortar, the mycelia were crushed with a pestle quickly. The crushed mycelia were put into Eppendorf tubes, and suspended by the addition of 0.3 ml of TE buffer [10 mmol/l Tris-HCl (pH 8.0), 1 mmol/l EDTA]. Further, 0.3 ml of lysis solution [2% SDS, 0.1 mmol/l NaCl, 10 mmol/l EDTA, 50 mmol/l Tris-HCl (pH 7.0)] was added to the suspension, and the solution was maintained at 37° C. for 30 min to perform lysis. The obtained lysate solution was centrifuged at 12,000×g, thereby collecting a supernatant. The supernatant was subjected in sequence to phenol treatment, ethanol precipitation, RNase treatment, phenol treatment (twice), chloroform treatment and then ethanol precipitation. Thus, chromosomal DNA was purified. Approximately 50 μg of chromosomal DNA was obtained by these procedures.

2 μg of the purified chromosomal DNA was digested with restriction enzyme BamH I at 37° C. for 2 hours. Then, the product was ligated to Lambda EMBL3-BamH I arm (STRATAGENE) using T4 ligase. The obtained phage DNA was packaged using Gigapack Gold (STRATAGENE), and then infected with Escherichia coli strain P2392 (STRATAGENE), thereby constructing chromosomal DNA libraries.

### EXAMPLE 4

### Isolation of DNA Encoding Nitrate Reductase Derived from Monascus purpureus

Nitrate reductase gene was isolated from the above chromosomal DNA library by a standard plaque hybridization method. As a probe for plaque hybridization, a 5 kb Hind III fragment of plasmid pND300 [Biosci. Biotech. Biochem., 59, 1795-1797 (1995)] comprising niaD gene derived from Aspergillus oryzae was used. To perform labeling of the probe, hybridization, and detection of signals, ECL Direct Nucleic Acid Labeling and Detection System (Amersham Pharmacia Biotech) was used. As a result, eight clones to which the above DNA probe hybridized were obtained from approximately 10,000 plaques.

Phage DNA was prepared by liquid culture from these positive plaques according to a standard method. Then, the DNA was digested with restriction enzyme BamH I, and then subjected to 0.8% agarose gel electrophoresis, thereby obtaining an about 12 kb DNA fragment. The fragment was subcloned to BamH I site of pUC 18 according to the standard method to construct pMA-niaD. Escherichia coli strain HB101 was transformed with pMA-niaD. pMA-niaD was prepared in large quantities, and then the nucleotide sequence of the DNA fragment subcloned was determined using a DNA sequencer (ABI377, Perkin Elmer).

As a result, the nucleotide sequence represented by SEQ ID NO: 1 comprising niaD gene derived from Monascus purpureus was obtained. The DNA sequence was compared with a previously reported sequence of niaD gene of Aspergillus oryzae (SEQ ID NO: 2) and the position of exon [Biosci. Biotech. Biochem., 59, 1795-1797 (1995): GenBank Accession No. D49701], so that the coding region of the protein was predicted for the DNA of Monascus purpureus (SEQ ID NO: 1). The nucleotide sequence of the coding region of the protein is shown in SEQ ID NO: 3; the amino acid sequence of nitrate reductase of Monascus purpureus that is encoded by this region is shown in SEQ ID NO: 4.

In addition, the obtained Escherichia coli transformant, Escherichia coli HB101/pMA-niaD comprising niaD gene derived from Monascus purpureus, was deposited on Mar. 2, 2000 at the International Patent Organism Depositary, National Institute of Advanced Industrial Science and Technology (Central 6, 1-1-1, Higashi, Tsukuba-shi, Ibaraki, Japan) (formerly, National Institute of Bioscience and Human-technology, Agency of Industrial Science and Technology, Japan, 1-1-3, Higashi, Tsukuba-shi, Tbaraki, Japan) under the accession No. FERM BP-7065.

### EXAMPLE 5

### Transformation Method of Filamentous Fungi Belonging to the Genus Monascus

(1) Preparation of Protoplast

100 ml of a nutrient medium (1% dextrin, 1% peptone, 1% yeast extract, 0.05% KH₂PO₄, 0.05% MgSO₄.7H₂O, pH 5.2) was dispensed into a 500 ml Erlenmeyer flask, and then the media were sterilized. 5 mm mycelial block was scraped with a inoculating loop from the plate media on which Monascus purpureus strain SN2-30-4 had been previously cultured, and then inoculated in the nutrient media. Static culture was performed at 30° C. for 4 to 5 days. The obtained culture solution was filtered with pre-sterilized G1 glass filter, thereby collecting the cells. Sterilized water was added to wash the cells, and then 0.5 g of wet cells was put into each of sterilized test tubes A and B. Next, 10 ml of protoplast preparation solution 1 [5 mg/ml lysing enzymes; Sigma, catalog number L1412, 2.5 mg/ml Sumizyme C (SHIN NIHON CHEMICAL CO., LTD), 0.8 mol/l NaCl, 100 mmol/l phosphate buffer, pH 6.0] previously sterilized by filtration was added to the test tube A; and 10 ml of protoplast preparation solution 2 [5 mg/ml lysing enzymes, Sigma, 0.8 mol/l NaCl, 100 mmol/l phosphate buffer, pH 6.0] previously sterilized by filtration was added to test tube B to suspend the cells. The cells were incubated at 30° C. for 3 hours while gently shaking, so that protoplasts were prepared.

(2) Purification of Protoplast

The above two types of enzyme reacted solutions were filtered respectively with G3 glass filter to remove mycelial fragments. Then, the protoplast suspension that had passed through the filter was centrifuged at 700×G to remove supernatant. 10 ml of 0.8 mol/l NaCl solution was added to the obtained protoplast for re-suspension, and then the mixture was centrifuged at 700×G. This procedure was repeated twice. Further, 10 ml of solution 1 [10 mmol/l CaCl₂, 0.8 mol/l NaCl, 10 mmol/l Tris-HCl buffer (pH 7.5)] was added to the precipitate for suspension. Then, the suspension was centrifuged at 700×G for washing the protoplast. The protoplast was suspended in solution 1, and then solution 2 [40% (w/v) PEG 4000, 50 mmol/l CaCl₂, 50 mmol/l Tris-HCl buffer solution (pH 7.5)] was added thereto, thereby preparing a protoplast solution. The protoplast solution consisted of three-fourths volume of solution 1 and one-fourth volume of solution 2, and had a protoplast density of 10⁸/ml.

(3) Transformation

20 μl of 0.5 to 0.8 μg/μl pMA-niaD plasmid DNA (hereinafter, all plasmid concentrations in transformation are 0.5 to 0.8 μg/μl) was added to two types of 200 μl of protoplast solutions obtained in (2) above, and then ice-cooled for 30 minutes. Next, 1 ml of solution 2 was added to the solutions, and then the solutions were allowed to stand at room temperature for 20 minutes. Next, 10 ml of solution 1 was added to dilute PEG concentration, and then the solutions were centrifuged at 700×G to collect precipitate. The resulting two types of precipitates were suspended separately in 200 μl of solution 1. The suspension was mixed with a 0.5% soft agar medium (the medium has the same composition as a plate medium described below except for the agar concentration), and then poured onto the basal plate medium containing 10 mmol/l NaNO₃ as a nitrogen source. The plate medium was incubated at 30° C. for 10 to 14 days, thereby obtaining transformants.

The transformation efficiency was 1 to 3 colonies/μg DNA when protoplast preparation solution 1 containing lysing enzymes (Sigma) and Sumizyme C (SHIN NIHON CHEMICAL CO., LTD) had been used to prepare protoplasts. However, no transformant was obtained when protoplast preparation solution 2 containing only lysing enzymes (Sigma) had been used to prepare protoplasts. Therefore, better results could be obtained when both lysing enzymes (Sigma) and Sumizyme C (SHIN NIHON CHEMICAL CO., LTD) were used for protoplast preparation in transformation.

Furthermore, chromosomal DNA was prepared from the above transformants, and then integration of the introduced pMA-niaD plasmid was confirmed by Southern hybridization. Southern hybridization was performed as follows. After digestion of 4 μg of chromosomal DNA of Monascus purpureus transformant using restriction enzyme Xba I, the product was separated by 0.8% agarose gel electrophoresis. The separated DNA was blotted on a nylon membrane filter by capillary blotting. Using ECL Direct Nucleic Acid Labeling and Detection System (Amersham Pharmacia Biotech), hybridization was performed with 100 ng of pUC18 plasmid as a probe according to the attached protocol of the kit.

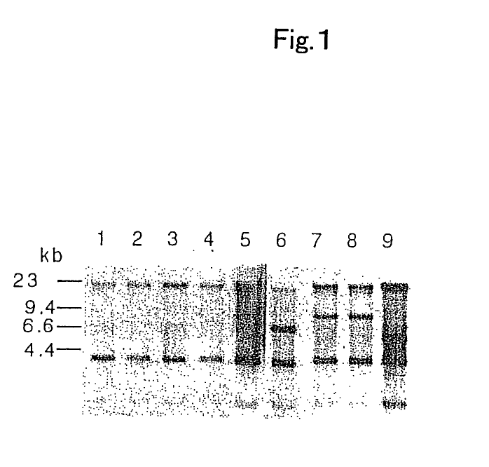

FIG. 1 shows the results. In FIG. 1, lanes 1 to 4 denote chromosomal DNA derived from transformants of Monascus purpureus strain SN2-30-4 into which pMA-niaD plasmid had been introduced. Lane 5 denotes chromosomal DNA derived from Monascus purpureus strain SN 2-30-4 used as a host. For the transformants (lanes 1 to 4), the hybridization bands were observed at around 20 kb in addition to the bands around 4 kb that were observed also for the host DNA (lane 5). Thus, it was confirmed that pMA-niaD plasmid had been introduced into the transformants.

### Example 6

### Preparation of Monascus purpureus Transformant into which Phytase Gene Derived from Aspergillus niger Introduced, and Secretory Production of Phytase by the Transformant

(1) Preparation of Protoplast

100 ml of a nutrient medium (1% dextrin, 1% peptone, 1% yeast extract, 0.05% KH₂PO₄, 0.05% MgSO₄.7H₂O, pH 5.2) was dispensed in a 500 ml Erlenmeyer flask, and then the solution was sterilized. 5 mm agar piece was excised using an inoculating loop from the plate media on which Monascus purpureus strain SN2-30-4 had been cultured. The piece was inoculated into the nutrient media, and then cultured statically at 30° C. for 4 to 5 days. The obtained culture solution was filtered with a pre-sterilized G1 glass filter, thereby recovering the cells. Further, after sterilized water was added for washing the cells, 0.5 g of wet cells was put into a sterilized test tube. Next, 10 ml of a filter-sterilized protoplast preparation solution [5 mg/ml lysing enzymes (Sigma), 2.5 mg/ml Sumizyme C (SHIN NIHON CHEMICAL CO., LTD), 0.8 mol/l NaCl, 100 mmol/l phosphate buffer, pH 6.0] was added to the tube, thereby suspending the cells. The cells were incubated while gently shaking at 30° C. for 3 to 5 hours, so that the protoplasts were dissociated.

(2) Purification of Protoplast

The above enzyme reacted solutions were respectively filtered with G3 glass filter to remove mycelial fragments. Then, the protoplast suspension that had passed through the filter was centrifuged at 700×G to remove supernatant. Further, 10 ml of 0.8 mol/l NaCl solution was added to the obtained precipitate for re-suspension, and then the suspension was centrifuged at 700×G. This procedure was repeated twice. Furthermore, 10 ml of solution 1 (10 mmol/l CaCl₂, 0.8 mmol/l NaCl, 10 mmol/l Tris-HCl buffer, pH 7.5) was added to the precipitate for suspension, and then the suspension was centrifuged at 700×G, thereby washing the protoplast. The protoplast was suspended in solution 1, and then solution 2 [40% (w/v) PEG 4000, 50 mmol/l CaCl₂, 50 mmol/l Tris-HCl buffer (pH 7.5)] was added thereto, thereby preparing a protoplast solution. The protoplast solution consisted of three-fourths volume of solution 1 and one-fourth volume of solution 2, and had a protoplast density of 10⁸/ml.

(3) Transformation

Ten μl of pMA-niaD plasmid and 10 μl of plasmid pANPHY1 (WO97/38096) comprising 4.6 kb of phytase gene phyA derived from Aspergillus niger were added to 200 μl of the protoplast solution obtained in (2), and then the solution was ice-cooled for 30 min. Next, 1 ml of solution 2 was added to the solution, and then allowed to stand at room temperature for 20 min. Then, 10 ml of solution 1 was added to dilute the protoplast solution, and then the solution was centrifuged at 700×G, thereby recovering precipitate. The precipitates were suspended respectively in 200 μl of solution 1. The suspension was placed on a basal plate medium containing 10 mmol/ NaNO₃ as a nitrogen source. Further, a 0.5% soft agar medium (the medium has the same composition as the above plate medium except for the agar concentration) was added to the suspension and mixed with it on the plate. Incubation was performed on the plate at 30° C. for 10 to 14 days, thereby obtaining transformants having both plasmids co-introduced therein.

Southern hybridization was performed using pUC18 as a probe according to the method described in Example 5 for 4 strains of the obtained transformants (strains named TF1, TF2, TF3 and TF4, respectively), 4 control strains of the transformants comprising only pMA-niaD introduced therein, and host strain SN2-30-4. (pANPHY1 is the plasmid constructed by inserting phyA gene into plasmid pUC118 derived from pUC18, and hybridizes with pUC18.) FIG. 1 shows the results. Lanes 1 to 4 denote chromosomal DNA samples extracted from the transformants comprising only selection marker plasmid pMA-niaD introduced therein; lane 5 denotes a chromosomal DNA sample extracted from host strain SN 2-30-4; and lanes 6 to 9 denote chromosomal DNA samples extracted respectively from the transformant TF1 to TF4 to which pANPHY1 plasmid comprising phyA and pMA-niaD plasmid co-introduced therein. For phyA gene-co-introduced transformants of lanes 6 to 9, novel hybridization bands could be detected at around 5 to 9 kb, while such bands were not detected for the transformants of lanes 1 to 4 comprising only pMA-niaD introduced therein. These results suggest that both plasmids, pANPHY1 comprising phyA gene and pMA-niaD, were successfully introduced.

(4) Production of Phytase by Transformant

The transformant strains TF1, TF2, TF3 and TF4 obtained in (3) were inoculated into a 500 ml Erlenmeyer flask containing 100 ml of a phytase production medium (3% sucrose, 10 mmol/l NaNO₃, 0.05% MgSO₄.7H₂O, 0.05% KCl, 0.1% corn steep liquor) and then subjected to static culture at 30° C. for 14 days. As a control, the transformants comprising only pMA-niaD introduced therein were cultured similarly. The cells were removed from the culture solution by filtration to obtain a culture supernatant, and the supernatant was used as a crude enzyme solution.

Phytase activity in the above crude enzyme solution was measured as follows. Enzyme activity was measured at 37° C., wherein 0.2 mol/l acetate buffer (pH 5.5) containing 2.5 mmol/l sodium phytate was used as a substrate, and a mixture of acetone: 2.5 mmol/l sulfuric acid: 10 mmol/l ammonium molybdate at 2:1:1 was used as a solution to stop reaction. After 0.5 ml of the substrate was incubated for 5 min, 0.5 ml of the crude enzyme solution was added to start the reaction. 10 minutes later, 2 ml of the solution to stop the reaction was added, and then the solution was stirred. Furthermore, the solution was mixed with 0.1 ml of 1 mol/l citric acid. Absorbance at 380 nm was measured by a spectrophotometer, so that the released phosphorus was determined. Under conditions of pH 5.5 and 37° C., enzymatic activity which isolates inorganic phosphorus at a rate of 1 μmol per minute was defined as 1 unit (U). As a result, as shown in Table 1, enzymatic activities of the transformants ranged from 1.4 to 18.0 mU/ml, which were 2 to 20 times greater than the control.

TABLE 1
Phytase activity of transformant

| Transformant | | Phytase activity |
|---|---|---|
| strain | Introduction plasmid | (mU/ml) |
| Control | pMA-niaD | 0.8 |
| TF1 | pMA-niaD, pANPHY1 | 18.0 |
| TF2 | pMA-niaD, pANPHY1 | 2.6 |
| TF3 | pMA-niaD, pANPHY1 | 1.4 |
| TF4 | pMA-niaD, pANPHY1 | 16.0 |

These results showed that the improved amount of secretory production of phytase was observed in the transformants of the filamentous fungus belonging to the genus Monascus in which plasmid pANPHY1 comprising phyA gene derived from Aspergillus niger had been introduced. This is because phyA gene of Aspergillus niger introduced in the transformant was expressed so that phytase of Aspergillus niger was produced by secretion.

### EXAMPLE 7

### Isolation of DNA Encoding Acetamidase Derived from Monascus purpureus

(1) Construction of a Chromosomal Library

In the same manner as in Example 3, 2 μg of chromosomal DNA purified from Monascus purpureus strain IFO30873 was digested with restriction enzyme EcoR I at 37° C. for 2 hours. Then, the digested product was ligated to lambda EMBL4-EcoR I arm (STRATAGENE) using T4 ligase. The resulting phage DNA was packaged using Gigapack Gold (STRATAGENE), and then infected with Escherichia coli strain P2392, thereby constructing a chromosomal DNA library.

(2) Isolation of DNA Encoding Acetamidase

DNA encoding acetamidase was isolated from the above chromosomal DNA library by a standard plaque hybridization method. An about 2.5 kb fragment of amdS gene [Gene, 108, 91 (1991)] derived from Aspergillus oryzae (the fragment had been amplified by PCR using genomic DNA of Aspergillus oryzae as a template and using sequences of SEQ ID NOS: 19 and 20 as DNA primers) was used as a probe for plaque hybridization. ECL Direct Nucleic Acid Labeling and Detection System (Amersham Pharmacia Biotech) was used to perform labeling of probes, hybridization and signal detection. As a result, 8 clones to which the above DNA probe hybridized were obtained from about 10,000 plaques.

Phage DNA was prepared by liquid culture according to a standard method from these positive plaques. The phage DNA was digested with restriction enzyme Sal I, and then an about 6.0 kb DNA fragment was obtained using 0.8% agarose. The fragment was subcloned to Sal I site of pUC18 according to a standard method, to construct pMA-amdS. Then, Escherichia coli strain HB101 was transformed with pMA-amdS. pMA-amdS was prepared in large quantities, and then the nucleotide sequence of the subcloned DNA fragment was determined using a DNA sequencer (ABI 377, Perkin Elmer).

The thus provided nucleotide sequence comprising amdS gene derived from Monascus purpureus is shown in SEQ ID NO: 5. The DNA sequence was compared with the previously reported sequence of amdS gene of Aspergillus oryzae (SEQ ID NO: 6) and the position of exon [Gene, 108, 91-98 (1991): GenBank Accession No. D10492], so as to assume the protein coding region of Monascus purpureus DNA of SEQ ID NO: 5. A nucleotide sequence of the protein coding region is shown in SEQ ID NO: 7, and an amino acid sequence of acetamidase of Monascus purpureus encoded by this region is shown in SEQ ID NO: 8.

In addition, the resulting Escherichia coli transformant HB101/pMA-amdS comprising amdS gene derived from Monascus purpureus was deposited under Accession No. FERM BP-7064 at the International Patent Organism Depositary, National Institute of Advanced Industrial Science and Technology, (Central 6, 1-1-1, Higashi, Tsukuba-shi, Ibaraki, Japan) (formerly, National Institute of Bioscience and Human-Technology, 1-1-3, Higashi, Tsukuba-shi, Ibaraki, Japan) on Mar. 2, 2000.

### EXAMPLE 8

### Isolation of DNA Encoding Alcohol Dehydrogenase II Derived from Monascus purpureus

(1) Construction of a Chromosomal Library

In the same manner as in Example 3, 2 μg of chromosomal DNA purified from Monascus purpureus strain IFO30873 was digested with restriction enzyme EcoR I at 37° C. for 2 hours. Then, the digested product was ligated to lambda DASHII-EcoR I arm (STRATAGENE) using T4 ligase. The resulting phage DNA was packaged using Gigapack Gold (STRATAGENE), and then infected with Escherichia coli strain P2392, thereby constructing a chromosomal DNA library.

(2) Isolation of DNA Encoding Alcohol Dehydrogenase II

DNA encoding alcohol dehydrogenase II was isolated from the above chromosomal DNA library by a standard plaque hybridization method. An about 0.8 kb fragment corresponding to position 1009 to 1776 of the nucleotide sequence of alcB gene [Curr. Genet., 29, 122-129 (1996), SEQ ID NO: 10] derived from Aspergillus nidulans (the fragment had been amplified by PCR using genomic DNA of Aspergillus nidulans as a template and using sequences of SEQ ID NOS: 21 and 22 as DNA primers) was used as a probe for plaque hybridization. ECL Direct Nucleic Acid Labeling and Detection System (Amersham Pharmacia Biotech) was used to perform labeling of probes, hybridization and signal detection. As a result, 6 clones to which the above DNA probe hybridized were obtained from about 10,000 plaques.

Phage DNA was prepared by liquid culture according to a standard method from these positive plaques. The phage DNA was digested with restriction enzyme EcoR I, and then subjected to 0.8% agarose gel electrophoresis, thereby obtaining about a 8.0 kb DNA fragment. The fragment was subcloned into EcoR I site of pUC18 according to a standard method to construct pMA-alcB. Then, Escherichia coli strain HB101 was transformed with pMA-alcB. pMA-alcB was prepared in large quantities, and then the nucleotide sequence of the subcloned DNA was determined using a DNA sequencer (ABI 377, Perkin Elmer).

The thus provided nucleotide sequence comprising alcB gene derived from Monascus purpureus is shown in SEQ ID NO: 9. The DNA sequence was compared with the previously reported nucleotide sequence of alcB gene of Aspergillus nidulans (SEQ ID NO: 10) and the position of exon [Curr. Genet., 29, 122-129 (1996): GenBank Accession No. Z48000], so as to assume the protein coding region of Monascus purpureus DNA of SEQ ID NO: 9. The assumed protein coding region is shown in SEQ ID NO: 11, and an amino acid sequence of alcohol dehydrogenase II of Monascus purpureus encoded by this region is shown in SEQ ID NO: 12.

In addition, the resulting Escherichia coli transformant JM110/pMA-alcB comprising alcB gene derived from Monascus purpureus was deposited under Accession No. FERM BP-7066 at the International Patent Organism Depositary, National Institute of Advanced Industrial Science and Technology, (Central 6, 1-1-1, Higashi, Tsukuba-shi, Ibaraki, Japan) (formerly, National Institute of Bioscience and Human-Technology, 1-1-3, Higashi, Tsukuba-shi, Ibaraki, Japan) on Mar. 2, 2000.

### EXAMPLE 9

### Transcriptional Regulation of alcB Gene Derived from Monascus purpureus

Monascus purpureus strain IFO30873 was shake-cultured at 28° C. for 4 days using potato-dextrose medium. Then the cells were recovered using 3G1 glass filter, and then washed with sterilized water. The cells were transferred into 10 mmol/l phosphate buffer (pH 6.0) and shaken at 28° C. for 24 hours, recovered using 3G1 glass filter, and then washed. Next, the cells were transferred respectively to induction media [2% carbon source (fructose, ethanol or glucose), 0.3% NaNO₃, 0.05% MgSO₄.7H₂O, 0.05% KCl, 0.1% KH₂PO₄, pH 5.5], and then cultured at 28° C. for 24 hours (shaking culture or static culture). Immediately after culturing, the cells were recovered using 3G1 glass filter, washed, and then frozen with liquid nitrogen. Total RNAs were prepared from the respective cells using ISOGEN (NIPPON GENE CO., LTD.) according to the protocols attached to the product. 20 μg of the obtained RNAs were applied to agarose gel containing formaldehyde, and then subjected to electrophoresis at 30V for 1 hour and then at 60V for 2 hours. The RNAs that had been subjected to electrophoresis were transferred to nylon membrane. According to a standard method, the RNAs were subjected to Northern hybridization using as a probe a 4 kb Hind III fragment of plasmid pMA-alcB comprising alcB gene derived from Monascus purpureus isolated in Example 8. Hybridization was performed at 65° C.

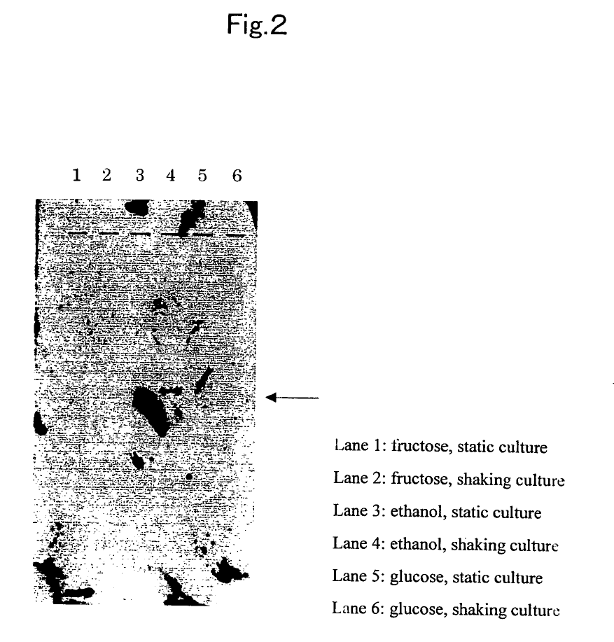

FIG. 2 shows the results. In FIG. 2, lane 1 denotes total RNA derived from Monascus purpureus strain IFO30873 that was statically cultured in a medium supplemented with fructose as a carbon source for 24 hours; lane 2 denotes total RNA derived from Monascus purpureus strain IFO30873 that was shake-cultured in a medium supplemented with fructose as a carbon source for 24 hours; lane 3 denotes total RNA of Monascus purpureus strain IFO30873 that was statically cultured in a medium supplemented with ethanol as a carbon source for 24 hours; lane 4 denotes total RNA derived from Monascus purpureus strain IFO30873 that was shake-cultured in a medium supplemented with ethanol as a carbon source for 24 hours; lane 5 denotes total RNA derived from Monascus purpureus strain IFO30873 that was statically cultured in a medium supplemented with glucose as a carbon source for 24 hours; and lane 6 denotes total RNA derived from Monascus purpureus strain IFO30873 that was shake-cultured in a medium supplemented with glucose as a carbon source for 24 hours.

As shown in FIG. 2, when a 4 kb fragment of alcB gene derived from Monascus purpureus was used as a probe, signals were detected for the RNA prepared from the cells that had been shake-cultured using ethanol as a carbon source. This result suggests that transcription of alcB gene is induced by ethanol. Since suppression of transcription of alcB gene of Aspergillus nidulans by ethanol was reported [Curr. Genet., 29, 122-129 (1996)], it was shown that alcB gene of Monascus purpureus and that of Aspergillus nidulans differ in their transcriptional regulation.

These results suggest that transcription of alcohol dehydrogenase II gene, alcB, of Monascus purpureus is induced by ethanol.

### EXAMPLE 10

### Isolation of DNA Encoding Acid Phosphatase Derived from Monascus purpureus

DNA encoding acid phosphatase was isolated by a standard plaque hybridization method from the chromosomal DNA library of Monascus purpureus strain IFO30873 obtained in Example 3. As a probe for plaque hybridization, a 1.5 kb fragment (amplified by PCR using genomic DNA of Aspergillus niger as a template and sequences of SEQ ID NOS: 23 and 24 as primers) of aph gene [Gene, 133, 55-62 (1993)] derived from Aspergillus niger (awamori) was used. ECL Direct Nucleic Acid Labeling and Detection System (Amersham Pharmacia Biotech) was used to perform labeling of probes, hybridization and signal detection. As a result, 5 clones to which the above DNA probe hybridized were obtained from an about 10,000 plaques.

Phage DNA was prepared by liquid culture according to a standard method from these positive plaques. The phage DNA was digested with restriction enzyme BamH I, and then about 10 kb DNA fragment was obtained by 0.8% agarose gel electrophoresis. The fragment was subcloned into BamH I site of pUC18 according to a standard method to construct pMA-aph. Then, Escherichia coli strain HB101 was transformed with pMA-aph. pMA-aph was prepared in large quantities, and then the nucleotide sequence of the subcloned DNA was determined using a DNA sequencer (ABI 377, Perkin Elmer).

The thus provided nucleotide sequence comprising aph gene derived from Monascus purpureus is shown in SEQ ID NO: 13. The nucleotide sequence was compared with the nucleotide sequence (SEQ ID NO: 14) of aph gene of Aspergillus niger (awamori) and the position of exon [Gene, 133, 55-62 (1993): GenBank Accession No. L02420], so as to assume the protein coding region of Monascus purpureus DNA of SEQ ID NO: 13. The assumed nucleotide sequence of the protein coding region is shown in SEQ ID NO: 15, and an amino acid sequence of acid phosphatase of Monascus purpureus encoded by this region is shown in SEQ ID NO: 16.

In addition, the resulting Escherichia coli transformant HB101/pMA-aph comprising aph gene derived from the genus Monascus was deposited under Accession No. FERM BP-7187 at the International Patent Organism Depositary, National Institute of Advanced Industrial Science and Technology, (Central 6, 1-1-1, Higashi, Tsukuba-shi, Ibaraki, Japan) (formerly, National Institute of Bioscience and Human-Technology, 1-1-3, Higashi, Tsukuba-shi, Ibaraki, Japan) on Jun. 15, 2000.

### EXAMPLE 11

### Expression of Human Lysozyme Gene in a Filamentous Fungus of the Genus Monascus

(1) Construction of Expression Plasmid of Human Lysozyme Gene

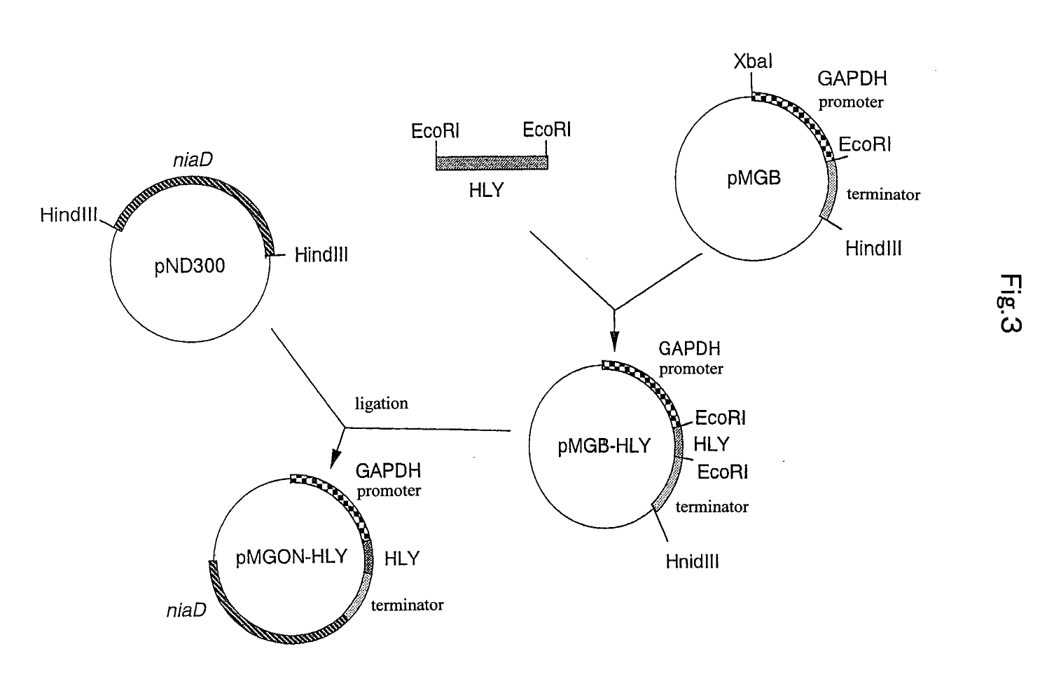

A construction process for expression plasmid pMGON-HLY of human lysozyme gene is shown in FIG. 3. At first pMGB vector having a promoter and a terminator of glyceraldehyde-3-phosphate dehydrogenase (hereinafter abbreviated as GAPDH) gene derived from Monascus purpureus was constructed. To obtain a promoter and a terminator of GAPDH gene by PCR, primers were designed based on the nucleotide sequence (GenBank Accession No. Z68498: the deposited sequence is shown in SEQ ID NO: 18. The region of gpd1 from position 1079 to 2346 of SEQ ID NO: 18 is assumed to encode GAPDH) of GAPDH gene, gpd1, of Monascus purpureus strain IFO4478 deposited with GenBank. PCR was performed using a DNA sequence of SEQ ID NO: 25 as a sense primer and a DNA sequence of SEQ ID NO: 26 as an antisense primer, and using the genomic DNA of Monascus purpureus strain IFO30873 as a template. Specifically, PCR was performed for 34 cycles, each cycle consisting of denaturation at 94° C. for 1 min, annealing at 50° C. for 2 min, and elongation at 72° C. for 3 min. Thus, a region corresponding to positions 1 to 1076 of SEQ ID NO: 18 comprising the promoter of GAPDH gene was amplified, and then Xba I site and Eco RI site were added to the 5′ side and 3′ side, respectively.

Further, PCR was performed using DNA of a nucleotide sequence of SEQ ID NO: 27 as a sense primer and DNA of a nucleotide sequence of SEQ ID NO: 28 as an antisense primer, and, as in the above amplification of the promoter section, using the genomic DNA of Monascus purpureus strain IFO30873 as a template. Specifically, PCR was performed for 34 cycles, each cycle consisting of denaturation at 94° C. for 1 min, annealing at 50° C. for 2 min, and elongation at 72° C. for 3 min. Thus, a region corresponding to positions 2350 to 2456 of SEQ ID NO: 18 comprising the terminator of GAPDH gene was amplified, and then EcoR I site and Hind III site were added to the 5′ side and 3′ side, respectively. These reaction solutions were subjected to electrophoresis, so that a 1.1 kb (promoter) fragment and a 0.1 kb (terminator) fragment were recovered and purified. The promoter fragment was digested with Xba I and EcoR I, and the terminator fragment was digested with EcoR I and Hind III. Subsequently, the digested fragments were inserted between Xba I-Hind III sites of vector pBluescript II SK (−) (STRATAGENE), thereby constructing plasmid pMGB. Determination of the nucleotide sequence of the above promoter section in pMGB revealed that it comprised a 1181 bp-long sequence shown in SEQ ID NO: 17 that differs partially from the sequence from positions 1 to 1076 of SEQ ID NO: 18. Further, comparison of the sequences of SEQ ID NO: 17 and SEQ ID NO: 18 showed insertion to 9 positions accounting for 105 nucleotides in total, and sequence substitution at 6 positions. Specifically, 9 insertions were found at position 405 (g), position 476 (g), a region from positions 680 to 770, position 779 (c), a region from positions 842 to 848, position 944 (c), position 966 (c), position 1030 (c), and position 1046 (c); and 6 sequence substitutions were found at position 76 (a→t), position 417 to 418 (cg→gc), position 851 to 852 (tt→cc), and position 855 (c→g).

Escherichia coli strain JM109 was transformed with pMGB. The thus obtained Escherichia coli transformant JM109/pMGB comprising the promoter and terminator of GDPDH gene derived from the genus Monascus was deposited on May 16, 2001 at the International Patent Organism Depositary, National Institute of Advanced Industrial Science and Technology, (Central 6, 1-1-1, Higashi, Tsukuba-shi, Ibaraki) (formerly, National Institute of Bioscience and Human-Technology, 1-1-3, Higashi, Tsukuba-shi, Ibaraki, Japan) under Accession No. FERM BP-7588.

To enable constitutive expression of a human lysozyme gene under regulation of the promoter derived from Monascus purpureus, pMGB-HLY was constructed by inserting a human lysozyme structural gene [Gene, 43, 273-279 (1986)] prepared to have EcoR I site at both of its ends and added with a chicken-lysozyme signal sequence to the EcoR I site existing at the junction between the promoter and terminator of GAPDH gene of pMGB plasmid. Next, a fragment comprising the full length niaD gene was excised by Hind III digestion from pND300 plasmid [Biosci. Biotech. Biochem., 59, 1795-1797, (1995)] comprising, as a selection marker gene for transformation of filamentous fungi of the genus Monascus, niaD gene derived from Aspergillus oryzae. The fragment was then inserted to Hind III site of pMGB-HLY, thereby constructing PMGON-HLY plasmid.

(2) Preparation and Purification of Protoplast

In the same manner as in Example 6 (1) and (2), a protoplast solution of Monascus purpureus strain SN2-30-4 was prepared.

(3) Transformation

20 μl of pMGON-HLY plasmid DNA prepared in (1) above was added to 200 μl of the protoplast solution prepared in (2) above, and then the solution was ice-cooled for 30 min. Subsequently, 1 ml of solution 2 was added to the solution, and then the solution was allowed to stand at room temperature for 20 min. Then, 10 ml of solution 1 was added to the solution to dilute PEG concentration. The solution was then centrifuged at 700×G, thereby recovering a precipitate. The precipitate was suspended in 200 μl of solution 1, and then the suspension was placed on a basal plate medium containing 10 mmol/l NaNO₃ as a nitrogen source. Further, 0.5% soft agar medium (the medium had the same composition as that of the above plate medium except for agar concentration) was added and mixed on the plate for inoculation. The above plate was incubated at 30° C. for 10 to 14 days, thereby obtaining transformants. As a result, it was confirmed that niaD gene of Aspergillus oryzae functions in filamentous fungi of the genus Monascus and can be used as DNA encoding a selection marker for transformation of filamentous fungi of the genus Monascus.

(4) Production of Human Lysozyme and Measurement of its Activity

A 5 mm agar piece was inoculated into an Erlenmeyer flask containing 25 ml of YPD medium (2% glucose, 2% poly peptone, 1% yeast extract) from the plate medium on which the transformants obtained in (3) had been cultured. After static culturing at 30° C. for 10 days, the cells were removed to obtain a culture supernatant. Using the supernatant as a crude enzyme solution, lysozyme activity was measured as follows. 800 μl of 50 mmol/l phosphate buffer (pH 6.4) and 5 μl of Micrococcus lysodeikticus suspension (20 mg/ml) were mixed in a microcell of a spectral photometer, and then 200 μl of the crude enzyme solution was added to start the enzyme reaction at 25° C. The activity was measured by measuring a decrease in absorbance at 450 nm. In this measurement, 1 unit was an enzyme level at which absorbance at 450 nm decreased by 0.001 for 1 min. In addition, protein content of lysozyme was calculated from activity measured when using 100,000 units/mg of specific activity of lysozyme [Biosci. Biotech. Biochem., 58, 1292-1296 (1994)]. As a result, the obtained transformants produced, at maximum, 20 μg/l human lysozyme. These results confirmed that DNA having the nucleotide sequence of SEQ ID NO: 17 functions as a promoter in filamentous fungi of the genus Monascus; and that the DNA can also be used for expression of the protein in filamentous fungi of the genus Monascus by inserting upstream of DNA encoding a desired protein.

### EXAMPLE 12

### Expression of Taka-Amylase A Gene Derived from Aspergillus oryzae in a Filamentous Fungus of the Genus Monascus

(1) Construction of Expression Plasmid of Taka-amylase A Gene

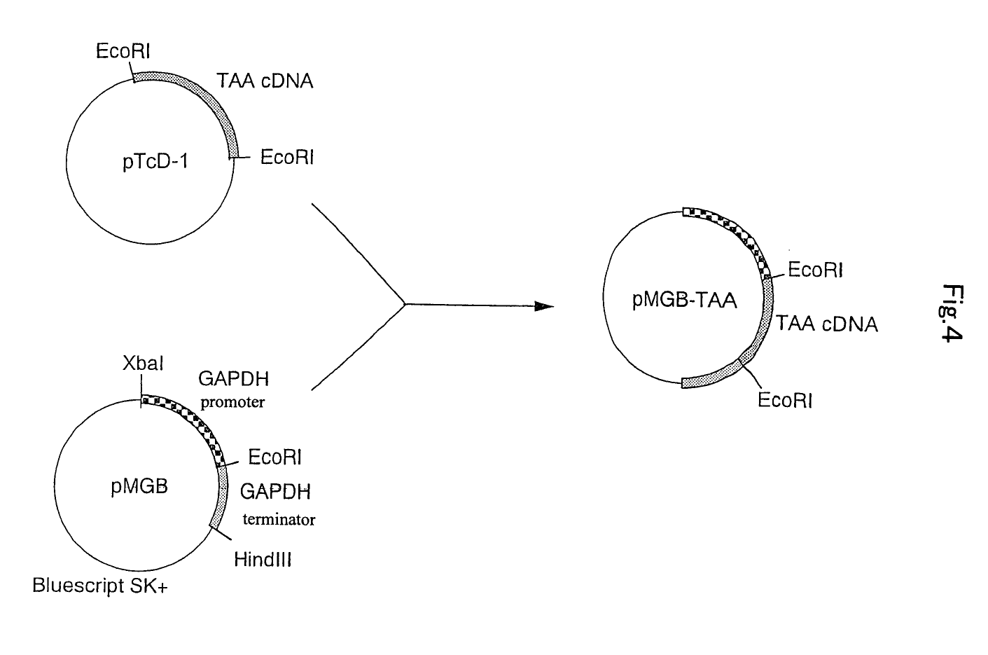

FIG. 4 shows a process for constructing expression plasmid pMGB-TAA of Taka-amylase A gene.

A fragment comprising cDNA of Taka-amylase A was excised by EcoR I digestion from plasmid pTcD-1 [Nagashima T. et al., Biosci. Biotech. Biochem., 56, 207-210 (1992)] comprising the cDNA of Taka-amylase A of Aspergillus oryzae. The fragment was then inserted to EcoR I site existing at the junction between GAPDH promoter and the terminator of pMGB plasmid, thereby constructing pMGB-TAA.

(2) Preparation and Purification of Protoplast

In the same manner as in Example 6 (1) and (2), a protoplast solution of Monascus purpureus strain SN2-30-4 was prepared.

(3) Transformation

Ten μl of pMGB-TAA plasmid prepared in (1) above and 10 μl of pMA-niaD plasmid comprising the selection marker gene, niaD, were added simultaneously to 200 μl of the protoplast solution prepared in (2) above, and then the solution was ice-cooled for 30 min. Subsequently, 1 ml of solution 2 was added to the solution, and then the solution was allowed to stand at room temperature for 20 min. Then, 10 ml of solution 1 was added to the solution to dilute PEG concentration. The solution was then centrifuged at 700×G, thereby recovering a precipitate. The precipitate was suspended in 200 μl of solution 1, and then the suspension was placed on a basal plate medium containing 10 mmol/l NaNO₃ as a nitrogen source. Further, 0.5% soft agar medium (the medium had the same composition as that of the above plate medium except for agar concentration) was added and mixed on the plate for inoculation. The above plate was incubated at 30° C. for 10 to 14 days, thereby obtaining transformants.

(4) Production of Taka-Amylase A and Measurement of its Activity

A 5 mm agar piece was inoculated into an Erlenmeyer flask containing 25 ml of DPC medium (2% dextrin, 0.3% peptone, 0.1% corn steep liquor, 0.05% MgSO₄.7H₂O) from the plate medium on which the obtained transformants had been cultured. After static culturing at 30° C. for 14 days, the cells were removed to obtain a culture supernatant. Amylase activity was measured using the supernatant as a crude enzyme solution in accordance with Official Methods of Analysis of National Tax Administration Agency, Japan (BREWING SOCIETY OF JAPAN, 1993), and the protein content was calculated using 2200 units/mg as specific activity [Biosci. Biotech. Biochem., 58, 1292-1296 (1994)]. As a result, the obtained transformant strain produced, at maximum, 104 mg/l Taka-amylase A.

### EXAMPLE 13

### Expression of Phytase Gene Derived from Aspergillus niger in a Filamentous Fungus of the Genus Monascus by Promoter of Alcohol Dehydrogenase II Gene

(1) Plasmid Construction for Expression of Phytase Gene by Promoter of Alcohol Dehydrogenase II Gene

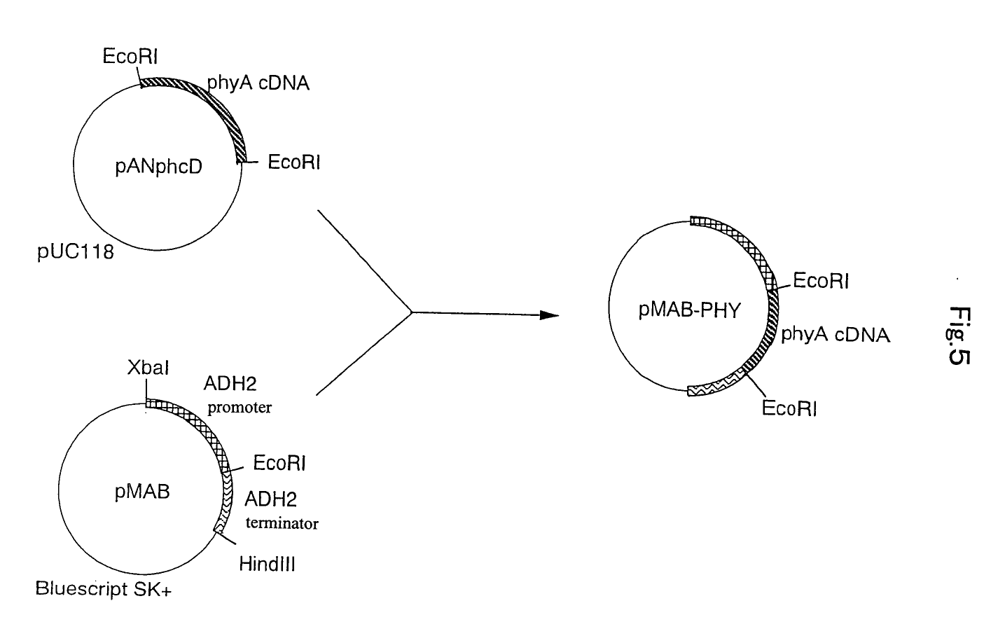

FIG. 5 shows a method for constructing expression plasmid pMAB-PHY of phytase gene. At first pMAB vector having the promoter and terminator of alcohol dehydrogenase II gene, alcB, derived from Monascus purpureus was constructed. To obtain the promoter and terminator of alcB gene by PCR, primers were designed based on the sequence (SEQ ID NO: 9) of alcB gene of Monascus purpureus. PCR was performed using a DNA sequence of SEQ ID NO: 29 as a sense primer and a DNA sequence of SEQ ID NO: 30 as an antisense primer, and using a 8 kb DNA fragment of alcB gene of Monascus purpureus comprising the sequence of SEQ ID NO: 9 obtained in Example 8 as a template. Specifically, PCR was performed for 35 cycles, each cycle consisting of denaturation at 94° C. for 1 min, annealing at 52° C. for 2 min, and elongation at 72° C. for 3 min. Therefore, the promoter region of alcB gene (a region comprising a sequence from position 1 to 611 of SEQ ID NO: 9) was amplified, and then BamH I site and EcoR I site were added to the 5′ end and 3′ end, respectively.

Further, PCR was performed using a DNA sequence of SEQ ID NO: 31 as a sense primer and a DNA sequence of SEQ ID NO: 32 as an antisense primer, and similar to amplification of the above promoter region, using a 8 kb DNA fragment of alcB gene of Monascus purpureus comprising the sequence of SEQ ID NO: 9 as a template. Specifically, PCR was performed for 35 cycles, each cycle consisting of denaturation at 94° C. for 1 min, annealing at 52° C. for 2 min, and elongation at 72° C. for 3 min. Thus, the terminator region of alcB gene was amplified. The reaction solution was subjected to electrophoresis, and then the 1.2 kb (promoter) and 0.3 kb (terminator) fragments were recovered and purified. The promoter fragment was digested using EcoR I and BamH I, and the terminator fragment was digested using EcoR I and Xho I. Both the fragments were inserted between BamH I-Xho I sites of pBluescriptSK (+) vector (STRATAGENE), thereby constructing pMAB vector.

Plasmid pANphcD prepared by subcloning cDNA of phyA from Aspergillus niger (WO 97/38096) to pUC118 (TAKARA SHUZO CO., LTD.) was digested using EcoR I. A fragment comprising cDNA of phyA was excised, and then inserted to EcoR I site existing at the junction between the promoter and terminator of alcB gene of pMAB vector, thereby constructing plasmid pMAB-PHY.

(2) Preparation and Purification of Protoplast

In the same manner as in Example 6 (1) and (2), a protoplast solution of Monascus purpureus strain SN2-30-4 was prepared.

(3) Transformation

Ten μl of pMAB-PHY plasmid prepared in (1) above and 10 μl of pMA-niaD plasmid comprising the selection marker gene, niaD, were added simultaneously to 200 μl of the protoplast solution prepared in (2) above, and then the solution was ice-cooled for 30 min. Subsequently, 1 ml of solution 2 was added to the solution, and then the solution was allowed to stand at room temperature for 20 min. Then, 10 ml of solution 1 was added to the solution to dilute PEG concentration. The solution was then centrifuged at 700×G, thereby recovering a precipitate. The precipitate was suspended in 200 μl of solution 1, and then the suspension was placed on a basal plate medium containing 10 mmol/l NaNO₃ as a nitrogen source. Further, 0.5% soft agar medium (the medium had the same composition as that of the above plate medium except for agar concentration) was added and mixed on the plate for inoculation. The above plate was incubated at 30° C. for 10 to 14 days, thereby obtaining transformants.

(4) Production of Phytase and Activity Measurement

A 5 mm agar piece was inoculated into an Erlenmeyer flask containing 25 ml of DPC medium (2% dextrin, 0.3% peptone, 0.1% corn steep liquor, 0.05% MgSO₄.7H₂O) from the plate medium on which the transformants obtained in (3) had been cultured. After static culturing at 30° C. for 14 days, the cells were removed to obtain a culture supernatant. Phytase activity was measured using the supernatant as a crude enzyme solution by the method described in Example 6, and the protein content was calculated using 150 units/mg [Appl. Environ. Microbiol., 65, 4682-4684 (1999)] as the specific activity of phytase. As a result, the obtained transformants produced, at maximum, 0.64 mg/l phytase.

### EXAMPLE 14

### Expression of Phytase Gene Derived from Aspergillus niger in a Filamentous Fungus of Monascus by Promoter of GAPDH Gene

(1) Plasmid Construction for Expression of Phytase Gene by Promoter of GAPDH Gene

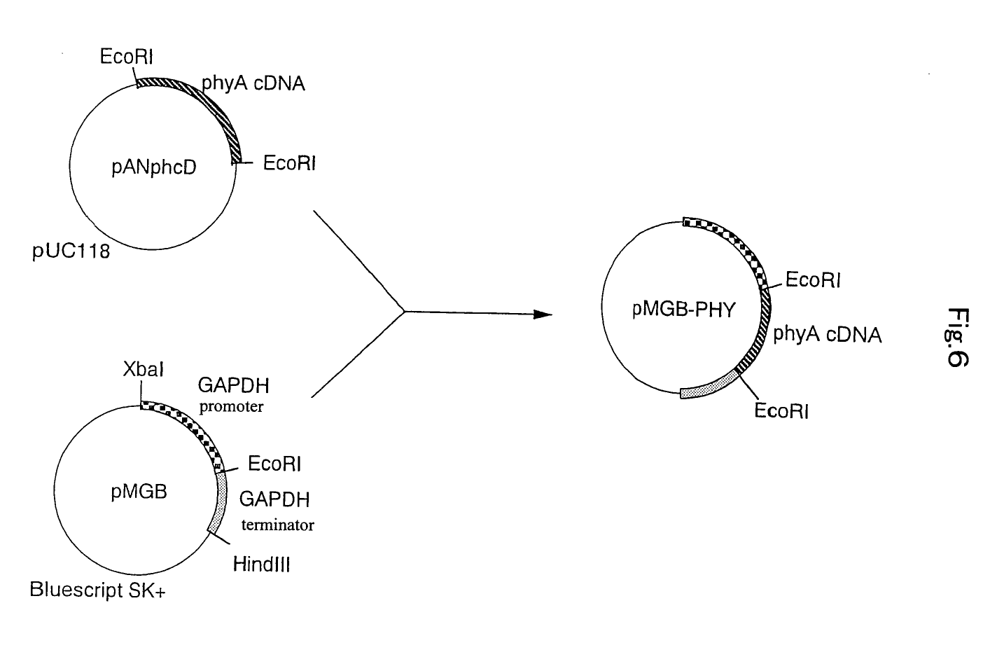

FIG. 6 shows a method for constructing expression plasmid pMGB-PHY of phytase gene. Plasmid pANphcD comprising cDNA of phyA from Aspergillus niger was digested with EcoR I, and a fragment comprising the cDNA of phyA was excised. The fragment was inserted at EcoR I site existing at the junction between the promoter and terminator of GAPDH gene of pMGB plasmid, thereby constructing pMGB-PHY.

(2) Preparation and Purification of Protoplast

In the same manner as in Example 6 (1) and (2), a protoplast solution of Monascus purpureus strain SN2-30-4 was prepared.

(3) Transformation

Ten μl of pMGB-PHY plasmid prepared in (1) above and 10 μl of pMA-niaD plasmid comprising the selection marker gene, niaD, were added simultaneously to 200 μl of the protoplast solution prepared in (2) above, and then the solution was ice-cooled for 30 min. Next, 1 ml of solution 2 was added to the solution, and then the solution was allowed to stand at room temperature for 20 min. 10 ml of solution 1 was then added to dilute PEG concentration. The diluted solution was centrifuged at 700×G to recover a precipitate. The precipitate was suspended in 200 μl of solution 1. The suspension was placed on a basal plate medium containing 10 mmol/l NaNO₃ as a nitrogen source, and then 0.5% soft agar medium (the medium had the same composition as that of the above plate medium except for agar concentration) was added and mixed on the plate for inoculation. The above plate was incubated at 30° C. for 10 to 14 days, so as to obtain transformants.

(4) Production of Phytase and Activity Measurement

A 5 mm agar piece was inoculated into an Erlenmeyer flask containing 25 ml of DPC medium (2% dextrin, 0.3% peptone, 0.1% corn steep liquor, 0.05% MgSO₄.7H₂O) from the plate medium on which the transformants obtained in (3) had been cultured. After static culturing at 30° C. for 14 days, the cells were removed, thereby obtaining a culture supernatant. Phytase activity was measured using the supernatant as a crude enzyme solution by the method described in Example 6. The protein content was calculated in the manner same as in Example 13. As a result, the obtained transformants produced, at maximum, 6.9 mg/l phytase.

### EXAMPLE 15

### Expression of Phytase Gene Derived from Aspergillus niger in Filamentous Fungi of the Genus Monascus by Promoter of Acid Phosphatase Gene

(1) Plasmid Construction for Expression of Phytase Gene by Promoter of Acid Phosphatase Gene

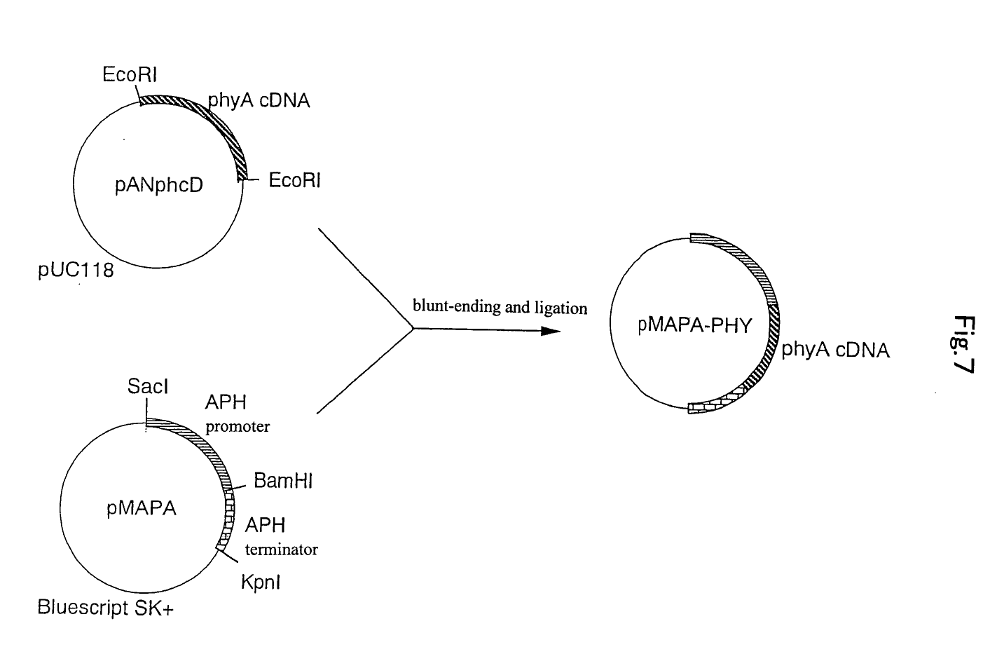

FIG. 7 shows a method for constructing expression plasmid pMAPA-PHY of phytase gene. First, vector pMAPA having a promoter and terminator of acid phosphatase gene aph derived from Monascus purpureus was constructed. To obtain the promoter and terminator of aph gene by PCR, primers were designed based on a nucleotide sequence (a region encoding acid phosphatase is present at positions 1014 to 2732 of SEQ ID NO: 13) of aph gene of Monascus purpureus. PCR was performed using a DNA sequence of SEQ ID NO: 33 as a sense primer, and a DNA sequence of SEQ ID NO: 34 as an antisense primer, and using a 10 kb DNA fragment of aph gene of Monascus purpureus comprising the sequence of SEQ ID NO: 13 obtained in Example 10 as a template. Specifically, PCR was performed for 35 cycles, each cycle consisting of denaturation at 94° C. for 1 min, annealing at 52° C. for 2 min, and elongation at 72° C. for 3 min. Thus, a region from positions 14 to 1013 of SEQ ID NO: 13 comprising the promoter of aph gene was amplified, and then Sac I site and BamH I site were added to the 5′ side and 3′ side, respectively. In addition, PCR was performed using a DNA sequence of SEQ ID NO: 35 as a sense primer, and a DNA sequence of SEQ ID NO: 36 as an antisense primer, and similar to amplification of the above promoter region, using a 10 kb DNA fragment of aph gene of Monascus purpureus comprising the sequence of SEQ ID NO: 13 as a template. Specifically, PCR was performed for 35 cycles, each cycle consisting of denaturation at 94° C. for 1 min, annealing at 52° C. for 2 min, and elongation at 72° C. for 3 min. Thus, a region from positions 2741 to 3011 of SEQ ID NO: 13 comprising the terminator of aph gene was amplified, and then BamH I site and Kpn I site were added to the 5′ side and 3′ side, respectively. The reacted solutions were subjected to electrophoresis, and then 1.0 kb (promoter) and 0.3 kb (terminator) fragments were recovered and purified. The promoter fragment was digested using Sac I and BamH I, and the terminator fragment was digested with Kpn I and BamH I. The digested products were inserted between Sac I-Kpn I sites of pBluescriptSK (+) vector, thereby constructing pMAPA vector.

Next, pMAPA vector was cleaved at BamH I site existing at the junction of the promoter and terminator of aph gene, and then blunt-ended. Then, a fragment comprising cDNA encoding phytase of Aspergillus niger as obtained by digesting pANphcD with EcoR I and allowing both ends of the digested pANphcD to be blunt-ended was inserted to the vector, thereby constructing pMAPA-PHY.

(2) Preparation and Purification of Protoplast

In the same manner as in Example 6 (1) and (2), a protoplast solution of Monascus purpureus strain SN2-30-4 was prepared.

(3) Transformation

Ten μl of pMAPA-PHY plasmid prepared in (1) above and 10 μl of pMA-niaD plasmid comprising the selection marker gene, niaD, were added simultaneously to 200 μl of the protoplast solution prepared in (2) above, and then the solution was ice-cooled for 30 min. Next, 1 ml of solution 2 was added to the solution, and then the solution was allowed to stand at room temperature for 20 min. 10 ml of solution 1 was then added to dilute PEG concentration. The diluted solution was centrifuged at 700×G to recover a precipitate. The precipitate was suspended in 200 μl of solution 1. The suspension was placed on a basal plate medium containing 10 mmol/l NaNO₃ as a nitrogen source, and then 0.5% soft agar medium (the medium had the same composition as that of the above plate medium except for agar concentration) was added and mixed on the plate for inoculation. The above plate was incubated at 30° C. for 10 to 14 days, so as to obtain transformants.

(4) Production of Phytase and Measurement of its Activity

A 5 mm agar piece was inoculated in an Erlenmeyer flask containing 25 ml of DPC medium (2% dextrin, 0.3% peptone, 0.1% corn steep liquor, 0.05% MgSO₄-7H₂O) from the plate medium on which the transformants obtained in (3) had been cultured. After static culturing at 30° C. for 14 days, the cells were removed, thereby obtaining a culture supernatant. Phytase activity was measured using the supernatant as a crude enzyme solution by the method described in Example 6. The protein content was calculated in the same manner as in Example 13. As a result, the obtained transformants produced, at maximum, 25 mg/l phytase. These results confirmed that DNA having a nucleotide sequence from positions 14 to 1013 of SEQ ID NO: 13 functions as a promoter in filamentous fungi of the genus Monascus; and that the DNA can also be used for expression of the protein in filamentous fungi of the genus Monascus by inserting the DNA upstream of a DNA encoding a desired protein.

### EXAMPLE 16

### Expression of Phytase Gene Derived from Aspergillus niger in a Filamentous Fungus of the Genus Monascus Using Signal Peptide of Acid Phosphatase

(1) Construction of a Plasmid for Expressing Phytase using a Signal Peptide of acid Phosphatase of Monascus purpureus

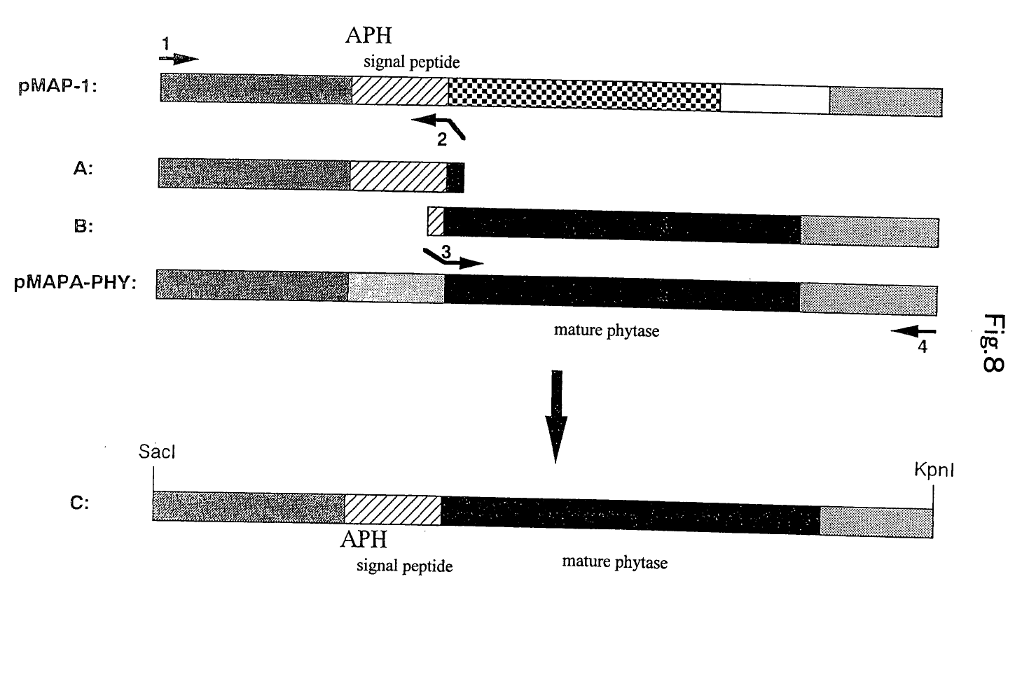

FIG. 8 shows a method for constructing phytase gene having the signal peptide of acid phosphatase. First, DNA encoding the signal peptide of acid phosphatase derived from Monascus purpureus was amplified by PCR as described below. PCR was performed using a DNA sequence of SEQ ID NO: 37 as sense primer 1, a DNA sequence of SEQ ID NO: 38 as antisense primer 2, and a 10 kb DNA fragment of aph gene of Monascus purpureus comprising the sequence of SEQ ID NO: 13 obtained in Example 10 as a template. Specifically, PCR was performed for 34 cycles, each cycle consisting of denaturation at 94° C. for 1 min, annealing at 52° C. for 1 min, and elongation at 72° C. for 2 min. The thus amplified DNA fragment (A) had Sac I site at its 5′ end and comprised the promoter and signal peptide encoding region of acid phosphatase gene of Monascus purpureus.

Further, PCR was performed using a DNA sequence of SEQ ID NO: 39 as sense primer 3, and a DNA sequence of SEQ ID NO: 40 as antisense primer 4, and using the plasmid pMAPA-PHY obtained in Example 15 for expressing phytase gene of Aspergillus niger as a template. Specifically, PCR was performed for 34 cycles, each cycle consisting of denaturation at 94° C. for 1 min, annealing at 52° C. for 1 min, and elongation at 72° C. for 2 min. The DNA fragment (B) comprising Kpn I site at its 3′ end, and comprising a region which encodes mature polypeptide of phytase was amplified. The reaction solution was subjected to electrophoresis, and then a 1.0 kb fragment [DNA fragment (A)] and a 1.5 kb fragment [DNA fragment (B)] were recovered and purified. Using both the purified fragments, sense primer 1 and antisense primer 4, PCR was performed for 34 cycles, each cycle consisting of denaturation at 94° C. for 1 min, annealing at 42° C. for 1 min, and elongation at 72° C. for 2 min. The DNA fragment (C) comprising Sac I site at its 5′ end, Kpn I site at its 3′ end, and a region that encodes mature phytase fused downstream of the promoter and signal peptide encoding region of acid phosphatase gene of Monascus purpureus was amplified.

The obtained fusion DNA fragment (C) was digested with Sac I and with Kpn I, and then inserted between Sac I-Kpn I sites of pUC18, thereby constructing pMAPA-aPHY.

(2) Preparation and Purification of Protoplast

In the same manner as in Example 6 (1) and (2), a protoplast solution of Monascus purpureus strain SN2-30-4 was prepared.

(3) Transformation

10 μl of pMAPA-aPHY plasmid prepared in (1) above and 10 μl of pMA-niaD plasmid comprising the selection marker gene, niaD, were added simultaneously to 200 μl of the protoplast solution prepared in (2) above, and then the solution was ice-cooled for 30 min. Next, 1 ml of solution 2 was added to the solution, and then the solution was allowed to stand at room temperature for 20 min. 10 ml of solution 1 was then added to dilute PEG concentration. The diluted solution was centrifuged at 700×G to recover a precipitate. The precipitate was then suspended in 200 μl of solution 1. The suspension was placed on a basal plate medium containing 10 mmol/l NaNO₃ as a nitrogen source, and then 0.5% soft agar medium (the medium had the same composition as that of the above plate medium except for agar concentration) was added and mixed on the plate for inoculation. The above plate was incubated at 30° C. for 10 to 14 days, so as to obtain transformants.

(4) Production of Phytase and Measurement of its Activity

A 5 mm agar piece was inoculated into an Erlenmeyer flask containing 25 ml of DPC medium (2% dextrin, 0.3% peptone, 0.1% corn steep liquor, 0.05% MgSO₄.7H₂O) from the plate medium on which the obtained transformants had been cultured. After static culturing at 30° C. for 14 days, the cells were removed, thereby obtaining a culture supernatant. Phytase activity was measured using the supernatant as a crude enzyme solution by the method described in Example 6. The protein content was calculated in the same manner as Example 13. As a result, the obtained transformants produced, at maximum, 16 mg/l phytase. These results confirmed that the signal peptide of acid phosphatase of Monascus purpureus can be used for secretory production of proteins other than acid phosphatase in filamentous fungi belonging to the genus Monascus.

### EXAMPLE 17

### Expression of Phytase Gene Derived from Aspergillus niger in a Filamentous Fungus of the Genus Monascus Using Signal Peptide of Taka-Amylase A

(1) Plasmid Construction for Expression of Phytase Gene Using Signal Peptide of Taka-Amylase A

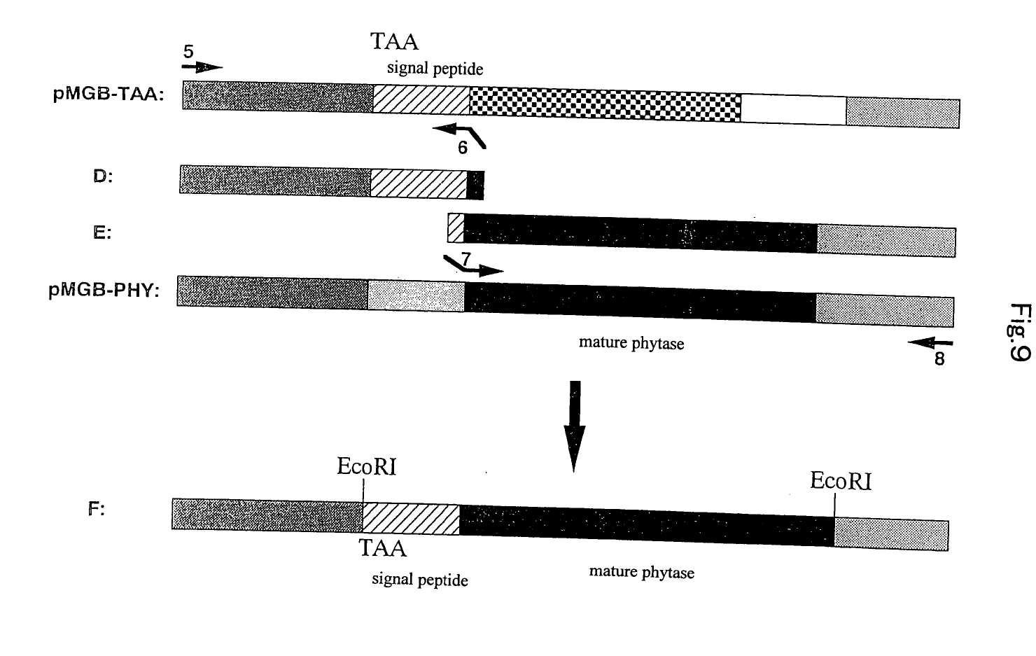

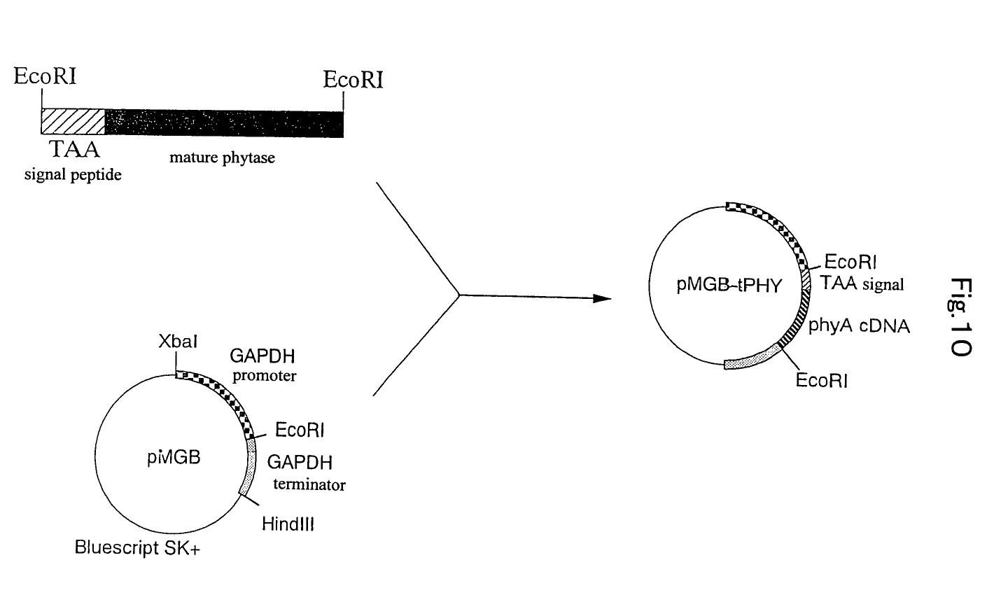

FIG. 9 shows a method for constructing phytase gene having the signal peptide of Taka-amylase A, and FIG. 10 shows a method for constructing pMGB-tPHY plasmid for expressing the gene. First, DNA encoding the signal peptide of Taka-amylase A derived from Aspergillus oryzae was amplified by PCR as follows. PCR was performed using a DNA sequence of SEQ ID NO: 41 as sense primer 5, and a sequence of SEQ ID NO: 42 as antisense primer 6, and using plasmid pMGB-TAA for expression of Taka-amylase A gene as a template. Specifically, PCR was performed for 34 cycles, each cycle consisting of denaturation at 94° C. for 1 min, annealing at 48° C. for 1 min, and elongation at 72° C. for 2 min. The DNA fragment (D) comprising a region encoding the signal peptide of Taka-amylase A was amplified.

Further, PCR was performed using a sequence of SEQ ID NO: 43 as sense primer 7 and a sequence of SEQ ID NO: 44 as antisense primer 8, and using plasmid pMGB-PHY for expression of phytase gene as a template. Specifically, PCR was performed for 34 cycles, each cycle consisting of denaturation at 94° C. for 1 min, annealing at 48° C. for 1 min, and elongation at 72° C. for 2 min. The DNA fragment (E) comprising a region which encodes mature polypeptide of phytase was amplified. The reaction solution was subjected to electrophoresis, and then a 1.1 kb fragment [DNA fragment (D)] and a 1.5 kb fragment [DNA fragment (E)] were collected and purified. Using both the purified fragments, sense primer 5 and antisense primer 8, PCR was performed for 34 cycles, each cycle consisting of denaturation at 94° C. for 1 min, annealing at 48° C. for 1 min, and elongation at 72° C. for 2 min. The DNA fragment (F) comprising a region which encodes the mature polypeptide of phytase fused downstream of a region encoding the signal peptide of Taka-amylase A was amplified.

The amplified DNA fragment (F) was digested with EcoR I (EcoR I site is present at the 5′ end of a region encoding the signal peptide of Taka-amylase A, and at the 3′ end of a region encoding the mature polypeptide of phytase), and then inserted at EcoR I site existing at the junction between the promoter and terminator of GAPDH gene of pMGB plasmid, thereby constructing pMGB-tPHY.

(2) Preparation and Purification of Protoplast

In the same manner as in Example 6 (1) and (2), a protoplast solution of Monascus purpureus strain SN2-30-4 was prepared.

(3) Transformation

10 μl of pMGB-tPHY plasmid prepared in (1) above and 10 μl of pMA-niaD plasmid comprising the selection marker gene, niaD, were added simultaneously to 200 μl of the protoplast solution prepared in (2) above, and then the solution was ice-cooled for 30 min. Next, 1 ml of solution 2 was added to the solution, and then the mixture was allowed to stand at room temperature for 20 min. 10 ml of solution 1 was then added to dilute PEG concentration. The diluted solution was centrifuged at 700×G to recover a precipitate. The precipitate was then suspended in 200 μl of solution 1. The suspension was placed on a basal plate medium containing 10 mmol/l NaNO₃ as a nitrogen source, and then 0.5% soft agar medium (the medium had the same composition as that of the above plate medium except for agar concentration) was added and mixed on the plate for inoculation. The above plate was incubated at 30° C. for 10 to 14 days, so as to obtain transformants.

(4) Production of Phytase and Measurement of its Activity

A 5 mm agar piece was inoculated into an Erlenmeyer flask containing 25 ml of DPC medium (2% dextrin, 0.3% peptone, 0.1% corn steep liquor, 0.05% MgSO₄.7H₂O) from the plate medium on which the obtained transformants had been cultured. After static culturing at 30° C. for 14 days, the cells were removed, thereby. obtaining a culture supernatant. Phytase activity was measured using the supernatant as a crude enzyme solution by the method described in Example 6. The protein content was calculated in the same manner as Example 13. As a result, the obtained transformants produced, at maximum, 50 mg/l phytase. These results confirmed that the signal peptide of Taka-amylase A of Aspergillus oryzae can be used for secretory production of proteins other than Taka-amylase A in filamentous fungi belonging to the genus Monascus, and that the production amount and secretion amount are high.

Table 2 summarizes plasmids used for expression of foreign genes in Examples 6 and 12 to 17 and the production amount of heterologous proteins.

TABLE 2

| | | | | Produc- |
|---|---|---|---|---|
| | | | | tion |
| Expression | | | Signal | amount |
| Plasmid | Promoter | Foreign gene | sequence | (mg/l) |
| pMGON-HLY | M. purpureus | human | chicken | 0.02 |
| | GAPDH | lysozyme | lysozyme | |
| pMGB-TAA | M. purpureus | A. oryzae | Same as | 104 |
| | GAPDH | Taka-amylase A | left | |
| pANPHY1 | A. niger | A. niger | Same as | 0.18 |
| | PhyA | Phytase | left | |
| pMAB-PHY | M. purpureus | A. niger | Same as | 0.64 |
| | alcB | Phytase | left | |
| pMGB-PHY | M. purpureus | A. niger | Same as | 6.9 |
| | GAPDH | Phytase | left | |
| pMAPA-PHY | M. purpureus | A. niger | Same as | 25 |
| | aph | Phytase | left | |
| pMAPA-aPHY | M. purpureus | A. niger | M. | 16 |
| | aph | Phytase | purpureus | |
| | | | Aph | |
| pMGB-tPHY | M. purpureus | A. niger | A. oryzae | 50 |
| | GAPDH | Phytase | Taka- | |
| | | | amylase A | |

### INDUSTRIAL APPLICABILITY

Using a filamentous fungus host belonging to the genus Monascus, red koji mold, which has been consumed as a food for a long time and is highly safe, we have developed reproducible transformation methods, including a method for introducing a gene and a method for selecting a recombinant strain, and thus established a novel efficient system for producing a useful substance. Further, we have isolated niaD gene and amdS gene derived from Monascus purpureus and shown their applicability as a selection marker used for transformation of filamentous fungi. We have also isolated alcB gene, aph gene and GAPDH gene having a novel promoter sequence derived from Monascus purpureus, and have shown the possibility of their application as a gene comprising a strong promoter and terminator to an expression system using a filamentous fungus host. These novel DNAs can be used not only for selecting a recombinant strain and expressing a recombinant DNA, but also as a gene for producing Monascus purpureus-derived nitrate reductase, alcohol dehydrogenase II, acetamidase and acid phosphatase.

According to the present invention, a method for expressing recombinant proteins at high levels using a filamentous fungus belonging to the genus Monascus as a host can be provided.

All publications, patents and patent applications cited herein are incorporated herein by reference in their entirety.

Sequence Listing Free Text

SEQ ID NO: 19-sense primer for amplification of Aspergillus oryzae amdS gene

SEQ ID NO: 20-antisense primer for amplification of Aspergillus oryzae amdS gene

SEQ ID NO: 21-sense primer for amplification of Aspergillus nidulans alcB gene

SEQ ID NO: 22-antisense primer for amplification of Aspergillus nidulans alcB gene

SEQ ID NO: 23-sense primer for amplification of Aspergillus niger aph gene

SEQ ID NO: 24-antisense primer for amplification of Aspergillus niger aph gene

SEQ ID NO: 25-sense primer for amplification of the promoter region of Monascus

purpureus GAPDH gene

SEQ ID NO: 26-antisense primer for amplification of the promoter region of Monascus

purpureus GAPDH gene

SEQ ID NO: 27-sense primer for amplification of the terminator region of Monascus

purpureus GAPDH gene

SEQ ID NO: 28-antisense primer for amplification of the terminator region of Monascus

purpureus GAPDH gene

SEQ ID NO: 29-sense primer for amplification of the promoter region of Monascus purpureus alcB gene

SEQ ID NO: 30-antisense primer for amplification of the promoter region of Monascus purpureus alcB gene

SEQ ID NO: 31-sense primer for amplification of the terminator region of Monascus purpureus alcB gene

SEQ ID NO: 32-antisense primer for amplification of the terminator region of Monascus purpureus alcB gene

SEQ ID NO: 33-sense primer for amplification of the promoter region of Monascus purpureus aph gene

SEQ ID NO: 34-antisense primer for amplification of the promoter region of Monascus purpureus aph gene

SEQ ID NO: 35-sense primer for amplification of the terminator region of Monascus purpureus aph gene

SEQ ID NO: 36-antisense primer for amplification of the terminator region of Monascus purpureus aph gene

SEQ ID NO: 37-sense primer for amplification of the promoter region and signal sequence of aph gene of Monascus purpureus

SEQ ID NO: 38-antisense primer for amplification of the promoter region and signal sequence of Monascus purpureus aph gene

SEQ ID NO: 39-sense primer for amplification of DNA encoding a mature polypeptide of Aspergillus niger phytase

SEQ ID NO: 40-antisense primer for amplification of DNA encoding a mature polypeptide of Aspergillus niger phytase

SEQ ID NO: 41-sense primer for amplification of DNA encoding a signal peptide of Aspergillus oryzae Taka-amylase A

SEQ ID NO: 42-antisense primer for amplification of DNA encoding a signal peptide of Aspergillus oryzae Taka-amylase A

SEQ ID NO: 43-sense primer for amplification of DNA encoding a mature polypeptide of Aspergillus niger phytase

SEQ ID NO: 44-antisense primer for amplification of DNA encoding a mature polypeptide of Aspergillus niger phytase

1. In a method for transforming filamentous fungi, the improvement comprising using a filamentous fungus belonging to the genus Monascus as a host.

2. The method according to claim 1, wherein the filamentous fungus belonging to the genus Monascus is Monascus purpureus.

3. The method according to claim 1 or 2, which comprises introducing into a host a recombinant DNA that is obtainable by incorporating into one vector a DNA encoding a marker for selecting a transformant and a DNA encoding a desired protein.

4. The method according to claim 1 or 2, which comprises introducing into a host two types of recombinant DNAs, one of which is obtainable by incorporating a DNA encoding a marker for selecting a transformant into a vector, and the other of which is obtainable by incorporating a DNA encoding a desired protein into a vector.

5. The method according to claim 3 or 4, wherein the DNA encoding a marker for selecting a transformant is selected from the group consisting of DNA encoding nitrate reductase of filamentous fungi, DNA encoding acetamidase of filamentous fungi, DNA encoding ornithine carbamyl transferase of filamentous fungi and DNA encoding orotidine -5′-phosphate decarboxylase of filamentous fungi.

6. The method according to claim 3 or 4, wherein the DNA encoding a marker for selecting a transformant is a DNA comprising a nucleotide sequence represented by any one of SEQ ID NOS: 1, 2, 3, 5 and 7.

7. The method according to claim 3 or 4, wherein the DNA encoding a marker for selecting a transformant is a DNA hybridizing to the DNA comprising the nucleotide sequence represented by SEQ ID NO: 1 or 3 under stringent conditions, and encoding a protein having activity substantially equivalent to nitrate reductase; or a DNA hybridizing to the DNA comprising the nucleotide sequence represented by SEQ ID NO: 5 or 7 under stringent conditions, and encoding a protein having activity substantially equivalent to acetamidase.

8. The method according to claim 3 or 4, wherein the recombinant DNA has a promoter which is located upstream of the DNA encoding a desired protein and is derived from a gene selected from the group consisting of an alcohol dehydrogenase gene, an acid phosphatase gene, a glyceraldehyde-3-phosphate dehydrogenase gene, a phosphoglycerate kinase gene, a glucoamylase gene, a phytase gene, a protease gene and a cellulase gene.

9. The method according to claim 3 or 4, wherein the recombinant DNA has a terminator which is located downstream of the DNA encoding a desired protein and is derived from a gene selected from the group consisting of an alcohol dehydrogenase gene, an acid phosphatase gene, a glyceraldehyde-3-phosphate dehydrogenase gene, a phosphoglycerate kinase gene, a glucoamylase gene, a phytase gene, a protease gene and a cellulase gene.

10. The method according to claim 8 or 9, wherein the alcohol dehydrogenase gene, the acid phosphatase gene or the glyceraldehyde-3-phosphate dehydrogenase gene is derived from the filamentous fungi belonging to the genus Monascus.

11. The method according to claim 10, wherein the alcohol dehydrogenase gene comprises the nucleotide sequence represented by SEQ ID NO: 9, the acid phosphatase gene comprises the nucleotide sequence represented by SEQ ID NO: 13, and the glyceraldehyde-3-phosphate dehydrogenase gene comprises the nucleotide sequence represented by SEQ ID NO: 17 or 18.

12. The method according to claim 8, wherein the promoter is capable of enhancing gene expression in the presence of lower alcohol.

13. The method according to claim 12, wherein the lower alcohol is ethanol or methanol.

14. The method according to claim 8, wherein the promoter comprises a DNA having a nucleotide sequence of 50 or more consecutive nucleotides between positions 1 and 615 of the nucleotide sequence of SEQ ID NO: 9.

15. The method according to claim 8, wherein the promoter comprises a DNA having a nucleotide sequence of 50 or more consecutive nucleotides between positions 1 and 1013 of the nucleotide sequence of SEQ ID NO: 13.

16. The method according to claim 8, wherein the promoter comprises a DNA having a nucleotide sequence of 50 or more consecutive nucleotides in the nucleotide sequence of SEQ ID NO: 17.

17. The method according to claim 8, wherein the promoter comprises a DNA having a nucleotide sequence of 50 or more consecutive nucleotides between positions 1 and 1078 of the nucleotide sequence of SEQ ID NO: 18.

18. The method according to claim 9, wherein the terminator comprises a DNA having a nucleotide sequence of 50 or more consecutive nucleotides between positions 1950 and 4142 of the nucleotide sequence of SEQ ID NO: 9.

19. The method according to claim 9, wherein the terminator comprises a DNA having a nucleotide sequence of 50 or more consecutive nucleotides between positions 2633 and 3831 of the nucleotide sequence of SEQ ID NO: 13.

20. The method according to claim 9, wherein the terminator comprises a DNA having a nucleotide sequence of 50 or more consecutive nucleotides between positions 2347 and 2456 of the nucleotide sequence of SEQ ID NO: 18.

21. The method according to claim 3 or 4, wherein the DNA encoding a desired protein comprises the nucleotide sequence represented by any one of SEQ ID NOS: 1, 3, 5, 7, 9, 11, 13 and 15.

22. The method according to claim 3 or 4, wherein the desired protein is selected from the group consisting of nitrate reductase, acetamidase, alcohol dehydrogenase II and acid phosphatase that are derived from filamentous fungi belonging to the genus Monascus, and phytase that is derived from Aspergillus niger.

23. The method according to claim 3 or 4, wherein the desired protein comprises an amino acid sequence represented by any one of SEQ ID NOS: 4, 8, 12 and 16.

24. The method according to claim 3 or 4, wherein the DNA encoding a desired protein is a DNA encoding a protein comprising a desired protein and a signal peptide of the secretory protein of a filamentous fungus which peptide has been added to the N-terminus of the desired protein.

25. The method according to claim 24, wherein the signal peptide of the secretory protein of the filamentous fungus is a signal peptide of phytase of Aspergillus niger, acid phosphatase of Monascus purpureus, or Taka-amylase A of Aspergillus oryzae.

26. A transformant of a filamentous fungus belonging to the genus Monascus, which is obtainable by any one of the methods according to claims 1 to 25.

27. A method for producing a protein, which comprises culturing the transformant according to claim 26, until a desired protein is produced and accumulated in a culture, and recovering the protein therefrom.

28. A DNA, which comprises a nucleotide sequence of 50 or more consecutive nucleotides between positions 1 to 615 of the nucleotide sequence of SEQ ID NO: 9.

29. A DNA, which comprises a nucleotide sequence of 50 or more consecutive nucleotides between positions 1950 and 4142 of the nucleotide sequence of SEQ ID NO: 9.

30. The DNA according to claim 28, which is capable of enhancing gene expression in the presence of lower alcohol.

31. The DNA according to claim 30, wherein the lower alcohol is ethanol or methanol.

32. A DNA, which comprises a nucleotide sequence of 50 or more consecutive nucleotides between positions 1 and 1013 of the nucleotide sequence of SEQ ID NO: 13.

33. A DNA, which comprises a nucleotide sequence of 50 or more consecutive nucleotides between positions 2633 and 3831 of the nucleotide sequence of SEQ ID NO: 13.

34. A DNA, which comprises the nucleotide sequence of SEQ ID NO: 17.

35. A recombinant DNA, which comprises as a selection marker a DNA comprising the nucleotide sequence represented by SEQ ID NO: 1 or 3 or a DNA which hybridizes under stringent conditions to a DNA comprising the nucleotide sequence represented by SEQ ID NO: 1 or 3 and encodes a protein having activity substantially equivalent to that of nitrate reductase.

36. A recombinant DNA, which comprises as a selection marker a DNA comprising the nucleotide sequence represented by SEQ ID NO: 5 or 7 or a DNA which hybridizes under stringent conditions to a DNA comprising the nucleotide sequence represented by SEQ ID NO: 5 or 7 and encodes a protein having activity substantially equivalent to that of acetamidase.

37. A recombinant DNA, which comprises as a promoter the DNA according to claim 28, 32 or 34.

38. A recombinant DNA, which comprises as a terminator the DNA according to claim 29 or 33.

39. A protein, which comprises an amino acid sequence represented by any one of SEQ ID NOS: 4, 8, 12 and 16.

40. A protein, which comprises an amino acid sequence wherein one or more amino acid residues are deleted, substituted and/or added in the amino acid sequence represented by any one of SEQ ID NOS: 4, 8, 12 and 16, and has activity equivalent to that of the protein comprising the amino acid sequence represented by any one of SEQ ID NOS: 4, 8, 12 and 16.

41. A DNA, which encodes the protein according to claim 39 or 40.

42. The DNA according to claim 41, which comprises a nucleotide sequence represented by any one of SEQ ID NOS: 1, 3, 5, 7, 9, 11, 13 and 15.

43. A DNA, which hybridizes to the DNA according to claim 42 under stringent conditions, and encodes a protein having activity substantially equivalent to that of a protein comprising an amino acid sequence represented by any one of SEQ ID NOS: 4, 8, 12 and 16.

44. An oligonucleotide, which comprises a nucleotide sequence that is identical to that of 15 to 60 consecutive nucleotides in a nucleotide sequence represented by any one of SEQ ID NOS: 1, 3, 5, 7, 9, 11, 13 and 15, or a nucleotide sequence that is complementary to that of the oligonucleotide.


SEQUENCE LISTING (44 sequences)

SEQ ID NO: 1 - DNA, 4527 bp, Monascus purpureus
  exon (1222)..(1498)
  exon (1574)..(1673)
  exon (1759)..(2017)
  exon (2072)..(2307)
  exon (2372)..(2595)
  exon (2658)..(3281)
  exon (3338)..(4239)
gggacagacgtttacctacatgaacgaaatggccaccattcccagcccaactaccacgatcctttggcgtctgccgccttcctggctctgctcgctgccattgtcgcggctgacgtgactgtctgaatcgctggattccccgattctatctaagccattgggagtattgccagcgttgccatggacaggcctaccgtccgtctgctcatccagtggcattgctgcaaagtgaaaacttgggtgaggatttcgggtgaatatttaggtaggtaggttcctacaaagagcatccagggagctccctcgctaataaacggagtatagcctgctaccgagtagactacagaaacgccgttggccaggcacaagatctctttgagaaccagcggaccacgcaattgcgcagccaggggaaccaggctgatgattcgcctgaagaatacggttgacattctgttctgggcatccagccttccttgccatgatttgcatctccgcggggagttggcatgaagataattacgcataatgaaggcgcaagcaaatcaatacgacacgtcttcctcggagatccgacttggaatcatgttagtgatgttgtttgcttatcagaccacatcgtgcagcagcgctttgccggctttcatcgcactgtttaggtttatttcatgccgtaaaggcgaaacctggcgatgctcctgaagccgttgtctcgccagaagagtatctgctatgctgtacggagggatttgctgtcaaatcccggctgtcatatcccggtgccgatactgttgattcaagccttctctttgcacttcgtttctgtcgggttcaaccgcatcgataatacggagaaatattagccattaaccaggggtcgccttatcgctgtgcagaatactctattctctgagcattccccgcggagatagtcaatgtgagtacggagtatatcacctccaagacgactgtggagacagatgtttccccaaaaatgatcatgatattgttcaattttcattacgggatgcttccgtttttcacatcaaatgttatgaaatcatttttgttccatttttcggcattggaactacaattagtcaatatacttgatccctgcacatacacggacatcctactctcagctacatggatctactagtttcgtgaagagcctccatcgctttaacagagtactgttctaattgcctctgtaaaaccatgggctccctcactcaagtccaaacggaaccggtcaccaccgcaacccattccatcgggtcagttcagttgaaggtcgaagaagactcctcgatgtcttctcctgatctagcggaaataatcccgctccctccgccatcgagagaaccctctgaggttttaaatatagacaagaacactcccgacggacacgttcctcgcgatcctcgactactgagacttacgggtgcgcatccgttcaattgtgagccaccgcttacggagctctacagccaaggtagttatatctttgagccttgttgtgtttcccctccgtagacaaggaaacgacgtctgatgtaccagggaaaaggttttctcacaccacccgaattgcattttgtcagaaatcacggacccgtcccccatgttcaggatgatgatattccggattgggaattaagcatcgaagggttagttgaccttgatgacatgtgtatttgtttctttagccttttgaaaaatgaagggggaaaaaatctgctaatcctcccgcagtcttgtggagagacccgttgtctggacattccaacagattttagacgaatttgagcaaataacggcccctgtcactctcgtatgtgctggtaatcgccgaaaggaacaaaatcaagtgcggaaaacgaaaggattttcctggggttctgcaggcatttctacgtcgctctacacaggacccttgatgggagacatcctacgacgagcaaagcctcttcgtcgagctaaatatgtatgcatggagggggctgatgtgttagtatgttatcattcttaagaaggaggaggacagaagaaaaactgactctcatagcctaatggacactatggcacatcgatcaagctcaactgggccctggatttcaatcgaggtatcatgctcgcccacaaaatgaacggggaacctctccgtccagatcacggtcgtcctttgcgcgttgttgttcccggccaaataggcggacgcagtgtcaaatggctgaagagactgatcctcacagactccccgagtacgaattggtatcatatcaatgacaaccgattgctaccgtgagttcttccacttcagatataatttgtttgcgggaacctgacaatatcgccaaatgtgaagaacgatggtctccccagagatggcatccgaagaccccaagtggtggcgagatgaccgatatgccatcttcgacctaaatgtcaattcttccgttgtatatccagaaaacaacgaggagcttgtgatagcttcagccccttcgacatataccgttaaaggatatgcttactccggtggtggccgacggattacaagagttgagatttccttggataagggaagatgtatgtacaagtgcatacgtatcccgaaaagtgaatttctgcctaactaattatcaatttagcatggcaccttgcgcacattgattacgccgaagacaagtatcgcaactttgaaggcgacctttttggcgggaaagtagacatgtactggcgggaaacttgcttctgctggtctttctggtcccttgacattccagtgtcggatctacaggctagtgatgccattttggtgcgggcaatggacgagtctttggctgttcaaccccgtgatatgtattggtctgtccttggaatgatgaacaatccgtggttccgcgtcaccatcacaaatgaaaacggaagattgaaatttgagcatccgacacacccaactaagactggtggctggatggaacgggtcaagaaagccggaggagatctggcgaacggttactggggagagacagttcaaggggaagcaccggcccagcaggagtctgcgaaagagataaatatgagaagggaagggctgagtaggctgatcgaactacaagagctcaaggatcatgttagcaatggagaaccttggtttatagtcaacggtgaggtatatgacggcactgaatttttaagggatcacccaggaggcgctcagagcatcatttcttctgccggaatggacgtttcggaggagttcctcgccattcgtaagtcgtctgaatggaaaccggcaagttatcacaagactaatattcgattatagacagtgaaactgcaaggattatgatgccgggttatcacatcggaacattaagcacatcagccctggctgttcttcaagataatggcctggaggaacagaacaactcaactgaacctcgcaagacatttctccagtctcgatactggtccaaaacaacactggtacggaagaagattgtatcctcggatagtcggatttttacgttcgagcttgaacatccaaaacagaccctgggtctaccagtcggccggcatcttatgatcagagtcccagacccaaccaagaagaacgagtgtatcatcagatcttatactcctatttctggtattacacaggagggaaccatggatatcctagtcaaagtttactttgatactgctacccaaccaggcggcaaaatgacaacggcccttgatagacttcccttgggctccacgatcgattgcaaaggtccaactggcaggtttgagtacctcggcaatggcaacatcctaataggtgaccaggagcgtcatgtcaagtccttccggatgatttgtggaggtagtggggtcacaccaatcttccaggttctgcgtgccgtgatgcaagatccggacgatccaacgacctgcgtggtgcttaatggaaacagacgcgaggaggacattctttgtcgggctgagctagacgctcttgtcgctctcaacaacgcaaaatgcaccatgattcacactttgaccaaggcgcctgagacatgggctggccatcgtggccgtatctccgagacgctgctgaaggaatatgctatgctcaatgacgactgtatggtgctggtttgtggtccagagagcatggagcgcgatgttcaaaagatactacttggtcttggatgggaagagtcgaatctacatttcttctaggatctttatttaccttcagataccatggatgggttagacgacatgagcccgaagagcatgcacgtatgttggatagagcggtatatactccggactacaactctaatatctgcatagaaatacatgaatgagagagccaggatggtcaatgtagttcattgatggcaatgatggaatgaccctaataatcccaatcggaagtcatttagggcatgtattacacgtgatcagagccaatcagggtcctgagatctctatcttcagttgcaagtttaagttcctcactga

SEQ ID NO: 2 - DNA, 5140 bp, Aspergillus oryzae
  exon (1200)..(1470)
  exon (1569)..(1668)
  exon (1719)..(1977)
  exon (2026)..(2261)
  exon (2320)..(2543)
  exon (2603)..(3226)
  exon (3283)..(4175)
aagcttaacaggccccaaattcaattaattgcacctgttattattagtcttactacaagtttgcatatcggcatctcaataaaaacccgcatactacgaaagggtctattacatcacgagctcttcggggtatggtgtgggtaccatccctgttcctcttaagaatagatgaaagggaaggtcatctcattccacccatgagacctacaattaagcactgtattggtatgtgaacgccagtctggtaaatcgcgcccccttgttgcctcaggtttcaccacaggtcataaatattgtacgtaagacaaatcctaagttagatgccgatatatccggcactcttcaagcatcatagcaatggcgttttaaatcacgcagttaggttggtgttcttctcatgtggtaaatcctcaagggtgtaactacaagtatacggtagacttccaagattggcaaaaaaagccagatcgagcgatttttgcctggatttaaaggatcctggagtgccaattaaacgtgagcaatatccctctaacaaacttaccgagaattcctttgaagggtacgtacagagtagtagctccgctctaacagccgtgagctcccatctggcccattctccacgcgaaccaggctcattcggacgataagcaacaacacttctaattggaagttgcacatggcttcacggcattgtctccatcattcttcaaagaaccaattaatatgacgaaaaagaaatcctgcaaggttgagcggacaaatggctggagcctcgagagtgttgtgtgggtcaacgcgatttatttgcctcaacgcttggttaagcggtgggacgccgtccagcctgaaggcttgccctaatttcgagcgtcccacctcccagaatgagctgattttgggactccgcggaaatcgctggtgggttcctaggagcattgcttattgtgacctttctccgaggcgttatatggtaacaaggagttactgccgtgacctaatagaatgtccactgcccgcgcatgggtaacacgtactgcgtgccatcatacgataggcaagagtattttagtgtggtggattgcctccttatttggagttaatgcactggctgaccattcgacttattcatactcagcatccttgttcatgctccctccctcgtaattttactttcattggccttccctgccggtaacatggcaaccatcaccgaggtgcggacggatgcgctcgtcccaactgacctcgttcttaagacaggtcagatcaaaatcgaaagcgaagagatctcgacgagagacctgtctgatatccctctgccaccgccatcaaaacggccgacagaagtgctgagcgtagataaaggaactccagatagccatgttcctcgtgaccctcggcttatcagattaacgggtgttcatccgtttaatgttgagccacctctcacagatctgtataaagaaggtatgagttataactgctccactcctatccttatcaggttgcttgcaccggctgtcatgcttgtccccttgagccgttacattctcacactctgaaagggtttttaacatcgccggagctcttctatgttcgaaatcatggcccagtccctcatatcaaggatgaagatatccctcactgggaaattagcatcgaagggttagtatagtgctaggttctctgccaaacatccgttaaccaaagtatagactggtagagaagcctttggtactaaacttccgacaagtgttgcagcagtacgaccaaataacagcgcctatcaccctcgtatgtgctggcaatcgacgcaaagagcaaaacaatgtacgtaaaacgaaaggtttttcctggggatcggcgggactatcgactgccctcttcactggcccattgctggcggatattctccgcagtgcgaaacccctgcgtaaagcgaaatacgtctgtatggaaggagcggataagctggtatgctgtacctctatcttatgatgataattgctaagttcgccgcagcccaatggtcactacggcacatctattaaattgaactgggccctggaccccaacagggggatcatgcttgcacataaaatgaacggggagtctcttcgcccagatcatggtcgtccgctgagggccgtcgtgcccggtcaaataggaggacgaagtgttaagtggctgaagaggctgatcttgaccgatgcaccaagcgacaactggtaccatatcaatgacaaccgcgtcttaccgtatgtctaaagggcacttattttatatttccatcatttgtctaactccctaacccagaacaatggtctcgcctgagatggcatcaaataaccgaaattggtggcacgatgagcggtatgcgatttatgacctaaacaccaactccgccgttgcatatccccaaaacaatgaggtcttaaatctcctggtcgcagggccgtcatatactgtcagaggatatgcatacgccggtgggggccgcagggttaccagggtagaaatatccctagacaaaggcaaatgtacgcaccctcgctcgctcgatgtgtgagaatgcttatcaaagctaacggacttatagcttggagattggcggaaatcgaatatgccgaagacaagtatcgtgattttgaaggcgagctttttggaggcaaagtagatatgtactggcgcgaaacttgcttctgctggtgtttttggtctctaagcatcaccatcccagagcttgagaacagtgatgccatccttgtaagagccatggacgaagcattgggcgtgcagcctcgcgatatgtactggtccgttctcggaatgatgaacaacccgtggttccgggttacaattacgaaggaaaacgggaacttgagattcgagcaccctacccaccctagtatgcctacaggatggatggaacgcgtcaaaaaggctgggggtgacctgacgaatggtaactggggagaaagacacgaaggagaggagccgacggagccggagcccgtgcaagacattaatatgaagaaagacgggctaagccgagtgattggttttgaagaattcaaggagaattcctgcgatgagaagccatggttcatcgtgaatggagaagtgtatgatggtcaagcatttcttgaaggccaccctggcggagcgcagagtattatctcctctgctggtctggatgtctctgaggaattccttgctattcgtgagtcccaaaaatatcacactgcaattgtaccatctattgacacctatccatagatagcgagacggcaaaggcgatgatgcctgagtaccatattggaacgatggatccggaaggtttaaaagcactcaaggatgatgcatcatcctccaccgatgaaattcgcccagtgttcctccaatcacggtcttggacaaaggcaacattgaaagaaaggaaagacatatcctgggatacacgaatatttagtttcaaattggaacacgaagatcaaacattgggtttaccagtcggccagcatcttatgatcaaagtcctcgacacatcatccaacaacgaagccatcatccgctcatacaccccaatttctgaaaccagccagaaagggaccgtggacttgctggttaaagtatactttgcaacagccacctcggcaggcggcaagatgacgatggccctggataggctgccattgggctccgtggtggaatgcaagggtccgacaggcagattcgaataccttggaaatggacgagttgtcataagtgggaaggaacgccatgttcggtcgtttaagatgatttgtggaggaaccggtatcacaccgatcttccaggtcttgcgcgccgtggttcaggaccggcaagatcccacctcttgtacagtcctcaatggaaacagacaggaggaagatatcctttgccgggctgagctcgacggcttcatggcaaccgacagcagaaggtgtaatataatacacaccctatccaaagcgccggactcatggactggccgccgaggacgcatatccgaagagctcctaaaggagtacgcggctccagaagatgagagtatggtcctgatttgtggtccgccagccatggaagaatcggctcggaggatactgttggcggaaggatggaaagaatcagaccttcacttcttctaaattgggattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcagtttcgtggcgcacgccgctcgttccatccatcctcgacaatggagcaaatgtcaaaatcgtaccgaaaatactttccagcagcttcgctgcatcagcatgtcttttgctgagaaagagcgcaaaaagcatttgatcgagaatatcttcatgataatctctaagtctagggacagaatgtgctgcttctatcgtgccatcaatatcaccgcggtcgaggcagcgttcaatcttagccaggctatcttggaaccgctgccaagtcgagccaatgccgacatgaaagcaataatcactcaatgagagcacgaaatgctggcagtcaatgcgaaatttctggtacacgtttcgagggtgcccagatagggagtctctccccgtagaatcacgaatgagacctttgacgaccgaaaccattcgaaggagtcgaagcagatgcttgaaaagacgatcatacttgttaagcgatcgcgacgtaatgatagcttccaggacgtctgatggtttgtattgaagacgtaggaaatccaaagctt

SEQ ID NO: 3 - DNA, 2622 bp, Monascus purpureus
atgggctccctcactcaagtccaaacggaaccggtcaccaccgcaacccattccatcgggtcagttcagttgaaggtcgaagaagactcctcgatgtcttctcctgatctagcggaaataatcccgctccctccgccatcgagagaaccctctgaggttttaaatatagacaagaacactcccgacggacacgttcctcgcgatcctcgactactgagacttacgggtgcgcatccgttcaattgtgagccaccgcttacggagctctacagccaaggttttctcacaccacccgaattgcattttgtcagaaatcacggacccgtcccccatgttcaggatgatgatattccggattgggaattaagcatcgaaggtcttgtggagagacccgttgtctggacattccaacagattttagacgaatttgagcaaataacggcccctgtcactctcgtatgtgctggtaatcgccgaaaggaacaaaatcaagtgcggaaaacgaaaggattttcctggggttctgcaggcatttctacgtcgctctacacaggacccttgatgggagacatcctacgacgagcaaagcctcttcgtcgagctaaatatgtatgcatggagggggctgatgtgttacctaatggacactatggcacatcgatcaagctcaactgggccctggatttcaatcgaggtatcatgctcgcccacaaaatgaacggggaacctctccgtccagatcacggtcgtcctttgcgcgttgttgttcccggccaaataggcggacgcagtgtcaaatggctgaagagactgatcctcacagactccccgagtacgaattggtatcatatcaatgacaaccgattgctaccaacgatggtctccccagagatggcatccgaagaccccaagtggtggcgagatgaccgatatgccatcttcgacctaaatgtcaattcttccgttgtatatccagaaaacaacgaggagcttgtgatagcttcagccccttcgacatataccgttaaaggatatgcttactccggtggtggccgacggattacaagagttgagatttccttggataagggaagatcatggcaccttgcgcacattgattacgccgaagacaagtatcgcaactttgaaggcgacctttttggcgggaaagtagacatgtactggcgggaaacttgcttctgctggtctttctggtcccttgacattccagtgtcggatctacaggctagtgatgccattttggtgcgggcaatggacgagtctttggctgttcaaccccgtgatatgtattggtctgtccttggaatgatgaacaatccgtggttccgcgtcaccatcacaaatgaaaacggaagattgaaatttgagcatccgacacacccaactaagactggtggctggatggaacgggtcaagaaagccggaggagatctggcgaacggttactggggagagacagttcaaggggaagcaccggcccagcaggagtctgcgaaagagataaatatgagaagggaagggctgagtaggctgatcgaactacaagagctcaaggatcatgttagcaatggagaaccttggtttatagtcaacggtgaggtatatgacggcactgaatttttaagggatcacccaggaggcgctcagagcatcatttcttctgccggaatggacgtttcggaggagttcctcgccattcacagtgaaactgcaaggattatgatgccgggttatcacatcggaacattaagcacatcagccctggctgttcttcaagataatggcctggaggaacagaacaactcaactgaacctcgcaagacatttctccagtctcgatactggtccaaaacaacactggtacggaagaagattgtatcctcggatagtcggatttttacgttcgagcttgaacatccaaaacagaccctgggtctaccagtcggccggcatcttatgatcagagtcccagacccaaccaagaagaacgagtgtatcatcagatcttatactcctatttctggtattacacaggagggaaccatggatatcctagtcaaagtttactttgatactgctacccaaccaggcggcaaaatgacaacggcccttgatagacttcccttgggctccacgatcgattgcaaaggtccaactggcaggtttgagtacctcggcaatggcaacatcctaataggtgaccaggagcgtcatgtcaagtccttccggatgatttgtggaggtagtggggtcacaccaatcttccaggttctgcgtgccgtgatgcaagatccggacgatccaacgacctgcgtggtgcttaatggaaacagacgcgaggaggacattctttgtcgggctgagctagacgctcttgtcgctctcaacaacgcaaaatgcaccatgattcacactttgaccaaggcgcctgagacatgggctggccatcgtggccgtatctccgagacgctgctgaaggaatatgctatgctcaatgacgactgtatggtgctggtttgtggtccagagagcatggagcgcgatgttcaaaagatactacttggtcttggatgggaagagtcgaatctacatttcttctag

SEQ ID NO: 4 - protein, 873 aa, Monascus purpureus
MGSLTQVQTEPVTTATHSIGSVQLKVEEDSSMSSPDLAEIIPLPPPSREPSEVLNIDKNTPDGHVPRDPRLLRLTGAHPFNCEPPLTELYSQGFLTPPELHFVRNHGPVPHVQDDDIPDWELSIEGLVERPVVWTFQQILDEFEQITAPVTLVCAGNRRKEQNQVRKTKGFSWGSAGISTSLYTGPLMGDILRRAKPLRRAKYVCMEGADVLPNGHYGTSIKLNWALDFNRGIMLAHKMNGEPLRPDHGRPLRVVVPGQIGGRSVKWLKRLILTDSPSTNWYHINDNRLLPTMVSPEMASEDPKWWRDDRYAIFDLNVNSSVVYPENNEELVIASAPSTYTVKGYAYSGGGRRITRVEISLDKGRSWHLAHIDYAEDKYRNFEGDLFGGKVDMYWRETCFCWSFWSLDIPVSDLQASDAILVRAMDESLAVQPRDMYWSVLGMMNNPWFRVTITNENGRLKFEHPTHPTKTGGWMERVKKAGGDLANGYWGETVQGEAPAQQESAKEINMRREGLSRLIELQELKDHVSNGEPWFIVNGEVYDGTEFLRDHPGGAQSIISSAGMDVSEEFLAIHSETARIMMPGYHIGTLSTSALAVLQDNGLEEQNNSTEPRKTFLQSRYWSKTTLVRKKIVSSDSRIFTFELEHPKQTLGLPVGRHLMIRVPDPTKKNECIIRSYTPISGITQEGTMDILVKVYFDTATQPGGKMTTALDRLPLGSTIDCKGPTGRFEYLGNGNILIGDQERHVKSFRMICGGSGVTPIFQVLRAVMQDPDDPTTCVVLNGNRREEDILCRAELDALVALNNAKCTMIHTLTKAPETWAGHRGRISETLLKEYAMLNDDCMVLVCGPESMERDVQKILLGLGWEESNLHFF

SEQ ID NO: 5 - DNA, 2948 bp, Monascus purpureus
  exon (414)..(677)
  exon (748)..(888)
  exon (945)..(1215)
  exon (1275)..(1424)
  exon (1473)..(1630)
  exon (1680)..(1829)
  exon (1886)..(2398)
ggatcccggatatgtctaagctgtttctctctttttttccccggctcccaagagggctactcttaattatagatgcatgcatcctggagtatagaccatgcaatgcgtatcacagcgctaccggcgctcatcaacctcaacgatccgacgccctgccgagcagcaacaggagataataattattaatgcgcggacaagaacagaagaacatccatgacaaatgtttctgaagcttatcatgccgcccgatacgccccgcatggcccctcttctaacattctaccctgcagctttaactgcccctgaaacccgaaaagcattaacataaacgccgtgtagacggttcaatcgacagcacttctactacagcgtacaatcatagttcggctttctcaaacgagaatagcacagcaatgaccgccacctgggaggaacgggctgccgataagagacaccggatcgagcagtcgattccggcggaatggaaggtcaagtcccttcccactgcagactctgttttcgattttccggagaagtcggggctgttatccgagaaggaacgggaaatcacacagtcttctgcgacggatctcgtggcgaagctggcgaagggagaactgaagtcggtggatgttactttggcgttttgtaagagggctgctttggcgcatcaattggtaggtcttccggctctccctgacggaatcatatatcagcagaaatggactctgctgatggacaaggcaggtcaactgtgttcttgaattcttcccggaagccgccctggcgcaggcgaaagggctcgatgcatattttgaggagcacaagaagcctgttgggccgctccatgggcttcctatttccctaaaggaccagttgcgaatcaaggtgcgatcgtgtgattatcgcaggacttggaatggttacattctaacgtggtccagggccttgaaacatccatgggatacgtctcctggctgggaaaatatgaaacccgtgattccatactcacggccctcctgcgcaaagccggcgcggttttctacgtgaagacaagcgtccctcaaactctgatggtatgcgagacaatcaacaacatcaccggtcgaactcttaacccgcggaacaagaattggtcgtgtggtggcagctctggaggcgagggcgccatggttggcatccgtggcagtattattggagttggcactgatattggtcagaatgctaccatttcgcaacgtcagccttgcagctgactgtgatttctgttctaggcggctctattcgggttccctctgcgttcaatttcctctacgggattcgaccgagccatggtaggatgccgtatggctacatggcgaacagtatggagggacaggagaccgtccatagcgttgttggtcccattgcgcattctgcttcgggtaagctgggtagccttattcgggcttgtattctctaacagattctagatctgagacttttcttgacgtcggttctcagcgaggaaccatggaagtatgactcgaaagtagttccgctaccgtggaggtctcatgaagaagaggccattcgcactaaactgcagtccgggggtcttacattggggttctttaattgcgacggaaacgtaagatagcatgaagtactggcaaacatcccttttaacaatcctacaggttcttccccatccaccagttctacgaggccttgatacggttgtctcggtgctgaagaagaatgggcacagcattgttccctggacaccatacaaacatgactttgccgttgacctgatctacggaatttattctgctgatggaagcacggtatgttatttttatcttgtttagtggaataccggagactgataggtgctactcaggacgtactacgagacatcaacgcctctggagagcccgctattccaaacatcaaagatctcctcaacccgagcgtcaggaaagccgacctaaacgatgtctggaatgtgcaactccagaaatggaagtatcagtgcgagtatctcgacaaatggcgtgtgctagaggaagagctcggcaaggagctcgatgccatcatagcgcccatcaccccgactgctgctattagacacaaccagttccggtactatggctacgcttcggtgatcaacttgctggacttcaccagcgtcgtggttcctgtgacgtttgcggacaagactatcgatgtgaagaatgaggagtataagccgttgaacgagctggatgcgctggtcaacaaggagtatgatgcggaggcgtaccatggcgcccccgtagcggtgcagattatcgggaggaagttgagtgaggaacggacattggcgattgcagaggaaataggaaggctattgggtaactctgtgacaccttgaaatagcattttgacgtttccggggtttggttcatggtggaggtattcctcaattatccttgtttccatgaaatgatacttttcacaggaatattgtatcgcttgatcgtttctatcagctatgggccatagaccaaccacactgtctcgaaactagctgtaaaatcagggttacgatatagaccacgtataagaacgcccggaaggaatgggacatgctgtcaatgttgatgcgtcttaaaatgtagagggccagtggagcacattcccctgcctccagagcccatggatgcagaaaatcctaaacacctggaccgctatatccaggtatatgaagccgatagatacatccggcgttatcagccccatcagctctacgctgcggggatgggagatcttattgcagctccatgaatcccgcctgatacgcccggtggatcgtcgaagacagaatcctcttgatttccgagacgaatgctttgtttgccatttgattctataaaagcacctcgagatgcggtattggcatgcaagcttggcactggccgtcg

SEQ ID NO: 6 - DNA, 2661 bp, Aspergillus oryzae
  exon (360)..(626)
  exon (678). .(818)
  exon (871)..(1141)
  exon (1194)..(1343)
  exon (1392)..(1549)
  exon (1603)..(1752)
  exon (1801)..(2301)
aatcagtctgtagaatgctggaccagcccgaaagcactcccaggcttggtgaatttataatggtaaagcaatggttatctcatgtgggtccagacaactcccagaaccacatggccaagataaacacgagataaccggttttatcgcccaattaaaagctaaaaagcctcacttatgcttccatttctccttgcgcggtccgtatggccgcgatgcaaagttgaggttgcggggtaaccccaaatttgagacttggagattggagatataaagcaacgtactccatggtcaatgttctgtggaaacaccagggtgattgctctaccatcgtctacagctgtatttatcactttgtcactatgccatctgccagctgggaagatctcgctgccgacaagagggcacgtttggagaagtccatccccgacgaatggaaattcaagtcagtcccaatagaaggctcggtcatcgatctacctgagaagtctgggattctgtcgccttctgaaataaagattacaaactcgtctgccacagaacttgtcgctcaattagccaatggcacgttgaagtccgtggatgtgacactcgcattctgtaaaagagctgcactggctcatcaacttgtgggtataaccttcgcctcgatcggagatacatgaaactaatgagaataggttaattgcgcacatgacttcttcccagagctagcactagcccaggccagggaacttgatcggtatttcgagacgcacaagaaacccgtgggaccattgcatggattaccgatttctttgaaagaccaattacgagtcaaggtaagacgagcttcctacactactgtgtgcatctcttctaacatagaactagggaactgaaacatgcatggcctatatctcttggctgggtaagcgcgacaccagcgattcgatattgactgccctcttgagaaaagcgggcgcagtattccttgttaagacgagtgtaccacaaacactgatggtatgtgagaccgtcaataatattatcggtcggacatcgaacccaaggaatctcaacctttcttgcggtggtagttcgggaggcgaaggtgccatgattgcaatgcgtggaggcgccatcggtataggaactgatatcggtagctatccatacttggttcatcagttattctggcgactaatgatatccaggtggatctattcgtgtcccagccgcattcaactccttgtatgggattcgtccaagtcacgatcgtctgccttacggtggtatgacgaacagcatggaaggtcaggaaacgatacacagcgtcgttggaccaattgcgcattctgctcaaggtagggatctgggatatttcttcgcgtcgagatactgatgctttctagatgtcagactcttccttcagtctgtccttaaggaggaaccttggaagtatgattcgaaagtcataccgcttccttggagggaggccgaggagaacgccgcccaagcaaaaattgctgagaagagtctaaatttcgcattttacgattttgatggcgttgtaagtattagtcgctcctcctccttcgcaatcatgcctgacagttggataaggtacgtcctcaccctccgattactcgtggcgttgagatcgtccggtctacgctcgagaaggacggacataccgtggcaccctggacaccctacaagcatgcatttgccgtagatttagccaacaaaatctacgctgcagatggaagcacggtaagtagcccccctaagaaaattagtatacgtgctaacatattgtaggatgtttacaagcacatcaacgcctcaggagaacccgctattccgaacatcaaggacctcatgaatcccaacctacccaaggcagatttgaatgaggtatgggacgcgcagctgcaaaaatggcgttatcagtgtgaataccttgacaagtggcgcgaatgggaggaacggacgggcaaggagcttgacgctatcatcgccccggtggcggcgacagctgcagtccgccacaaccaattccggtactatgggtatgctactgtctttaacgtgttagattacaccagtgttgttgtcccggttacctatgcagacaaggcggtggatcacagattggcggattatcagccggttagtgatatggataaggcggtttatgcggagtatgatcccgaggtttatcatggcgcacccgttgccgtgcagattatcggcagacgtcttagtgaggagcggaccctggctattgcggagtatgttgggaagttgttaggtcactagctttcaagctcagttttagccactacttgaagcgtctttgtcgagtgagactactgtaattagcataccatttacgaggccacgccgagtatttcgccattcaaagcagcttatacgtacctatgggttgttctgatatatgggtagcttataatctccactcctcgtattccgaagatacccaaaaatcctttacaatctgtctaatgccttgctgaatggcgccacatccacaaggtcggtgtcatctttcgagccgtaatgtttctaccaaggatgtactaatgacactccttgacagcttagacctactacgcacgcctcgttatgtgtgtcagagacaaataagcactatctaga

SEQ ID NO: 7 - DNA, 1647 bp, Monascus purpureus
atgaccgccacctgggaggaacgggctgccgataagagacaccggatcgagcagtcgattccggcggaatggaaggtcaagtcccttcccactgcagactctgttttcgattttccggagaagtcggggctgttatccgagaaggaacgggaaatcacacagtcttctgcgacggatctcgtggcgaagctggcgaagggagaactgaagtcggtggatgttactttggcgttttgtaagagggctgctttggcgcatcaattggtcaactgtgttcttgaattcttcccggaagccgccctggcgcaggcgaaagggctcgatgcatattttgaggagcacaagaagcctgttgggccgctccatgggcttcctatttccctaaaggaccagttgcgaatcaagggccttgaaacatccatgggatacgtctcctggctgggaaaatatgaaacccgtgattccatactcacggccctcctgcgcaaagccggcgcggttttctacgtgaagacaagcgtccctcaaactctgatggtatgcgagacaatcaacaacatcaccggtcgaactcttaacccgcggaacaagaattggtcgtgtggtggcagctctggaggcgagggcgccatggttggcatccgtggcagtattattggagttggcactgatattggcggctctattcgggttccctctgcgttcaatttcctctacgggattcgaccgagccatggtaggatgccgtatggctacatggcgaacagtatggagggacaggagaccgtccatagcgttgttggtcccattgcgcattctgcttcggatctgagacttttcttgacgtcggttctcagcgaggaaccatggaagtatgactcgaaagtagttccgctaccgtggaggtctcatgaagaagaggccattcgcactaaactgcagtccgggggtcttacattggggttctttaattgcgacggaaacgttcttccccatccaccagttctacgaggccttgatacggttgtctcggtgctgaagaagaatgggcacagcattgttccctggacaccatacaaacatgactttgccgttgacctgatctacggaatttattctgctgatggaagcacggacgtactacgagacatcaacgcctctggagagcccgctattccaaacatcaaagatctcctcaacccgagcgtcaggaaagccgacctaaacgatgtctggaatgtgcaactccagaaatggaagtatcagtgcgagtatctcgacaaatggcgtgtgctagaggaagagctcggcaaggagctcgatgccatcatagcgcccatcaccccgactgctgctattagacacaaccagttccggtactatggctacgcttcggtgatcaacttgctggacttcaccagcgtcgtggttcctgtgacgtttgcggacaagactatcgatgtgaagaatgaggagtataagccgttgaacgagctggatgcgctggtcaacaaggagtatgatgcggaggcgtaccatggcgcccccgtagcggtgcagattatcgggaggaagttgagtgaggaacggacattggcgattgcagaggaaataggaaggctattgggtaactctgtgacaccttga

SEQ ID NO: 8 - protein, 548 aa, Monascus purpureus
MTATWEERAADKRHRIEQSIPAEWKVKSLPTADSVFDFPEKSGLLSEKEREITQSSATDLVAKLAKGELKSVDVTLAFCKRAALAHQLVNCVLEFFPEAALAQAKGLDAYFEEHKKPVGPLHGLPISLKDQLRIKGLETSMGYVSWLGKYETRDSILTALLRKAGAVFYVKTSVPQTLMVCETINNITGRTLNPRNKNWSCGGSSGGEGAMVGIRGSIIGVGTDIGGSIRVPSAFNFLYGIRPSHGRMPYGYMANSMEGQETVHSVVGPIAHSASDLRLFLTSVLSEEPWKYDSKVVPLPWRSHEEEAIRTKLQSGGLTLGFFNCDGNVLPHPPVLRGLDTVVSVLKKNGHSIVPWTPYKHDFAVDLIYGIYSADGSTDVLRDINASGEPAIPNIKDLLNPSVRKADLNDVWNVQLQKWKYQCEYLDKWRVLEEELGKELDAIIAPITPTAAIRHNQFRYYGYASVINLLDFTSVVVPVTFADKTIDVKNEEYKPLNELDALVNKEYDAEAYHGAPVAVQIIGRKLSEERTLAIAEEIGRLLGNSVTP

SEQ ID NO: 9 - DNA, 4142 bp, Monascus purpureus
  exon (616)..(740)
  exon (827)..(875)
  exon (936)..(1092)
  exon (1177)..(1949)
aagcttacactttcacttctaggacatggaaaagccagcccctggagtaacgctcgcgacaacatgcatagatacatcacaccatgcacatgcacagttgaaacacataccttacgtatttcctgagcttccgcatctgcttcccacggtaccgcacgaagatctgccaacccaccacgtaaccgaggtgctgcttgcagtcacaaactgccgacagctcgggacaacccctttccccgcagtactacctccgcattccccagcaagaccgactttgaccagtggcatgatatcattcatgtgcatgcagaggagtgtaggagtacagctgaagggctggtgacccctcccagcccccgacaggaagatgcaggagagctgagtcgatcgagaacactgggaggggccggtacaaagtacggaatactggctctgtctggagatataaaatgaactataaaatgccggtcatttcccccaattgactcagaactgggctatcagaagttttactacctttatcgaactgcctgcatttctctaactgtacttctgtttgcaggctttcatatcatctgcgtctttataccttcttctcattgttcactcttcaacatggctgaacctcaaatcccaaccaagcagaaagctgcgatctatgataaaccgggtacggtctccaccaaagttgtggagatagatgtgcctgaacccggaccgggagaagttcttgtcaatttgtatgtcttgtacacccagtcaatgccctcatgccttgcgaattattattgagagtatacatatagctgactacaggatgctgcaggactcactcgggtatatgccactcggattatggtgtcatgacaaactctgttcgtcttcatctatactgcgcatacactgtagtccaattctaacaatgatatcaccagtggaaactgctcccctacccaacccagcccggacagattggcggccatgaaggagtcggaaaggtagtcaagttcggccctggagccaatggaaccgggttgaagattggcgatagggtaggaatcaaatgggtgtccagtgcatgcgggaactgtcgtacgtccggccttcatttcttatgaaaaggaaaaggaaaaatcctttctcttggaccaattcattaaatacaataatacacagacccatgccacgcaggcgcagacggaatctgcctcaaccagaaaatctccggctactacaccccgggcacattccaacaatacgcgacaggcccagctaactacgtaaccccgatccccgaaaacctctcttcggccgaggccgcgccccttctctgcgccggcgtcaccgtctacgcagccctgaaacgcagcaaagcccaaccaggccagtggattgtgatttccggcgcagacggcggcctgggccacctagccgtccaaatagccagtcgaggcatgggcctgcgcgtgatcggcatcgaccacggcagtaaagcagccctcgttaaggaatcaggtgcggagcatttcgtcgatatcaccgcattcccgaaagacgacaatggcgctgccatcgcggcacacgtgaagtccctcacgacagagaagttgggggcccacgccgttatcgtctgcacggcgtcaaacgcggcgtacgcgcaggcatttctcttcctgcgcttcaatggtacgctggtctgtgtggggatgccggaacatgagtcccaggctattgctacggcatatccggctgccatcgttttcaaccaggctactattaccggttcggcagtggggaatcggatagaggcgattgaggtgctagattttgccgctaggggtattatcaaatctcatgttaggatggctaagttggaggatttgacagatttgtttaatgagatggcagagggaaagttgcaggggagagtggtcttggatctttcttagttgggttggatggttgacgagatgggaaacagtcaattttatgattgaaattgattaattgatttgtacctgaatgtcttggggtttgcctataaggagtagtttattaaggaaaccatataactaactagcagcccagtcaaggtaaaagcaagataacaggcgacagcaacacaaagaaaaagaaggcaaggtttcagcgcgaaaagcactaacgcatgccatatcaatcttcatcaaatccacgagcccgtttactgtcctctcgaaacgaagctagagctgggtgtttctgcgcagaactcgaggaagcctctgcgcccgactcagcaacatggatcagggtagccagcttgcgatgtacagctcaggagcctgattgacctcaatcctgcgcaggatatcctttaacttggacgtgtactgacggagatccgacgtagggagcaattcaaggacatcgagaatgaagaacaggagcctcatctggccttttctcagatccgataagctgatggtacaagttgcgatggtcttcctcttcgccgtgaaatgtggctcggccccacatgttaccgtccagagcgtccttgttgcgattgctgatatcctccagcaaggtgacaagggctgttgtggctcccccggtgccatatgggaatcgggctagaatgttttctcggatacgatggacgttgtaccggatttgtttgatgacgttgctgacgtcgacgacaacgacacttgttgcgccgtccgtgggtaactggcctgtctggcagtatcagtcgaaatcggagagtaatttctgtgatttctgcagaatcttgtcgaagtagatagctgcgcaggctccagcgggcattgccttggagaggctgaagtagacgtcgtcgtgcacggctagtcgaaacatggtggcttcgaagtctccttgaacgacgcactgctctggcttacgaacttgtctcgctatcgctaccaagtcaggacggaaatcttcagggagggtgattttgttgaacgcagacatgatgtctcgcgctctgtccgatctgcttaaacacgagaaaccctccgtttcactgtcaacatattgtcaatttaattgatctgtgactactcccagcttgccatctaggagttgttcaatgtgtgagaaccctggagtatatccattgctcgacaagggaacggcagtggtagggggttgggccaggagacagccatggggttgatcgagaagccattatcaacgggaagaaatcagttcttcatttctcttccccttgccaacatgcacggagagggactgtacttccaaaaatgtgctgaaaaggcacgctgggccgattcaagaagaccgcacaagtgcatttatacccatccggctgatagtaaggacctgcggccgctccgacagtcgaaagccataccctatatgcatggcgcgacagcccacaacgtcgaattgggaagtaaggccgacgagagctttggacattataagtaatgccggatcttgctagaactctcgacaatttagtttcttcgctggtattatcatttctcgggccggaagtctctacacactgcatgttgatctggattaaaaggtctcggaactgtcttgttggagtagacgtagcgagaaatgattaggagggtttgctattttcgtagaccccgagaaggtcctatcccggtatgtataagggcctcatgggattcatggtccctaacgtgagcagcgtatgaaccaaggtatagctggtaatgagtactcacaagctattgaatagatttagagtaaattcgggattctcagtcaaatggcatgatcaggatagggtgcatttcccccgaggcgtaggacatgaagccaaatttacagacccagaccctgcctcattaggtttcttgtgcagtgaagggccaatgaacactgagaaatagcaatgacggcattatcatgcccgggacctattccgacaaacaggatgaaaattagccgatgccccttgggaactgtgaacaatccaacttggccatattgcctacatttcatcattggttgcacggcaaaaagtcatagacaagagcgcttaagaagcgtagaactaaatcaacgaataccagtccttccagatatgcaaaggaaaagtatcgtatccatatagatgacccaagctt

SEQ ID NO: 10 - DNA, 2190 bp, Aspergillus nidulans
  exon (776)..(900)
  exon (999)..(1047)
  exon (1114)..(1270)
  exon (1325)..(2097)
tggccataggcattacggcttgacatcattggcatagacttgttcaagaaggttcattatatagtgagtgacgcttggagtccagggtatctcacttcgacgcgatataaagtcagaccttgtcctagacatcgtctgcggtcctgctatggttcctcctcctggacccccgcgttttccccgccatcacatcgagcaattcgacaccaagttccgcagcgtttccgtcccaaccttgaacctgtcggatcccattggcgaagaccacattacgaacactctcaagtcagctagtttactgttacggactcaaaagactatgaaacgcgtcttatttgtctaccgtaaccgaggctgttccaccacggcacctcctcggccgccggcgcccctcacgctgcctcgagactgattggatgaatcatgataccacgttccatgaatgcgatattctagcttagctgcgtcatgctgaaacttcgctctctcagactcctgctttgggctggcctaaggcccactgctctctcctccttggatcagccactctcccctccctgcggagatgccctttcccctcattttcatatcagcaatcagactacctgactgcttctaaggttatggtcaggctagagatgatagtcgacttggcgttacgttgctctggtctgggatagccatataaagagaggcttgtccttcctctacttctttcaccttcccacatttatcttatcaacgatacaagagaagccccgcgaaatctgtcagaatggctgctcctgaaatccccaagaagcaaaaggctgtcatctacgacaaccccggtaccgtctctaccaaggtcgtcgagctggatgtacctgagcccggcgacaatgaagtcctgatcaatctgtgcgtgccctggatatacctgttaccttggaggaatgggatatccaagccgagtcgagtactggcatgtaccaattactaacgttataacattgtagcactcattccggcgtttgccactcagattttggtattatgaccaacacggtaggcacttagttcctgtcctggagtctctatcaagggcgaacgcatgctaatggtatgatacagtggaagatactacccttccctactcagcccggacaagtcggtggccatgaaggcgttggcaaagtggtgaagctcggcgcgggcgctgaagcatcaggattgaagatcggggacagagtcggtgttaagtggatttccagcgcctgtgggcagtgccgtacgtacatacaccttagacgcattttaaccaaaacttgctaacaacgcaaagctccatgccaggacggcgccgacggcctctgcttcaaccaaaaggtatcaggttactacacccctggcacattccagcaatacgtgctcggtcctgcgcaatacgttaccccaattcccgatggcctcccatcagccgaagcggcgccccttctctgtgccggtgtcacagtctacgcttctcttaagcgcagtaaagcccaaccaggtcaatggatcgtcatctccggcgctggcggcggccttggccacttagccgtccagatcgcagccaagggcatgggcctgcgtgtgattggcgttgaccacgggagtaaagaagagctcgtcaaggcgtcaggcgccgagcacttcgtggatatcaccaagttcccaacgggcgataaattcgaggccatctcctcgcacgtcaaatcgcttacaacgaagggtcttggtgcgcatgctgtcatagtttgcacggcgtccaatattgcttacgctcagtctttgctcttcctccggtacaacggaacgatggtctgcgtgggtatccccgagaacgagccgcagcgtatcgcaagtgcgtacccaggcctgtttatccagaagcatgtgcatgtcactgggtcggctgtcggaaataggaacgaggcgattgagactatggagtttgcggcgaggggtgtcattaaggcgcacttccgggaggagaagatggaggccttgactgaaattttcaaagagatggaggaggggaagttgcaggggcgggtggtgcttgatctttcttagtagctcttagcttggatgccctgcatggcctgacttgttatgtttatttcaatgttaacttctcttgaaatatgctcttgttagtcaaccctt

SEQ ID NO: 11 - DNA, 1104 bp, Monascus purpureus
atggctgaacctcaaatcccaaccaagcagaaagctgcgatctatgataaaccgggtacggtctccaccaaagttgtggagatagatgtgcctgaacccggaccgggagaagttcttgtcaatttgactcactcgggtatatgccactcggattatggtgtcatgacaaactcttggaaactgctcccctacccaacccagcccggacagattggcggccatgaaggagtcggaaaggtagtcaagttcggccctggagccaatggaaccgggttgaagattggcgatagggtaggaatcaaatgggtgtccagtgcatgcgggaactgtcacccatgccacgcaggcgcagacggaatctgcctcaaccagaaaatctccggctactacaccccgggcacattccaacaatacgcgacaggcccagctaactacgtaaccccgatccccgaaaacctctcttcggccgaggccgcgccccttctctgcgccggcgtcaccgtctacgcagccctgaaacgcagcaaagcccaaccaggccagtggattgtgatttccggcgcagacggcggcctgggccacctagccgtccaaatagccagtcgaggcatgggcctgcgcgtgatcggcatcgaccacggcagtaaagcagccctcgttaaggaatcaggtgcggagcatttcgtcgatatcaccgcattcccgaaagacgacaatggcgctgccatcgcggcacacgtgaagtccctcacgacagagaagttgggggcccacgccgttatcgtctgcacggcgtcaaacgcggcgtacgcgcaggcatttctcttcctgcgcttcaatggtacgctggtctgtgtggggatgccggaacatgagtcccaggctattgctacggcatatccggctgccatcgttttcaaccaggctactattaccggttcggcagtggggaatcggatagaggcgattgaggtgctagattttgccgctaggggtattatcaaatctcatgttaggatggctaagttggaggatttgacagatttgtttaatgagatggcagagggaaagttgcaggggagagtggtcttggatctttcttag

SEQ ID NO: 12 - protein, 367 aa, Monascus purpureus
MAEPQIPTKQKAAIYDKPGTVSTKVVEIDVPEPGPGEVLVNLTHSGICHSDYGVMTNSWKLLPYPTQPGQIGGHEGVGKVVKFGPGANGTGLKIGDRVGIKWVSSACGNCHPCHAGADGICLNQKISGYYTPGTFQQYATGPANYVTPIPENLSSAEAAPLLCAGVTVYAALKRSKAQPGQWIVISGADGGLGHLAVQIASRGMGLRVIGIDHGSKAALVKESGAEHFVDITAFPKDDNGAAIAAHVKSLTTEKLGAHAVIVCTASNAAYAQAFLFLRFNGTLVCVGMPEHESQAIATAYPAAIVFNQATITGSAVGNRIEAIEVLDFAARGIIKSHVRMAKLEDLTDLFNEMAEGKLQGRVVLDLS

SEQ ID NO: 13 - DNA, 3831 bp, Monascus purpureus
  exon (1014)..(1806)
  exon (1866)..(1983)
  exon (2039)..(2140)
  exon (2197)..(2632)
ttagtcctgggggatcattggaatgccgcagtaatcacagagtgagtacagagtaacactgtcaagcattgagaataatatgctctccggttgagaggggagcgatagttcgttagttacatcgctgtggcttatgtaatcacaccgggtgcgatgccacggttcaatccctcccaagttccaactgtggattcgacaatttcagctttccacagttcagctctacaggatcgatccatcgccaaggattccccggatcgatcggtgcggtggttcgtgagaacaaactcaatcgtatctaacaagtccagccagctaattcccgcggtagcaggattactcaataaatcccaacgaaaaacgtgaatatttattgtccttttcgaagagaaaaacagcagtattgtcaatacaaaccgactgaaataccttcgcgaggacacggtggactggcgcaggtattctggagcaagtgaggccctacgcccgagatacatcgatcatcccactgacagcggtttctagaagtgtcataaacacctcgcccattacgcacgtgggcacaagaggcacggtgaaaggccttgtggaccgtctgctgctgcaaggggccgtcgaaggatgaacctccggagatgagctggtggcttctcgtccggatcgcgtcggctttgccgtgcaaagcttgttccgtggggctgttatcaatcaatcgagtcccgtctgaccagcgccctattcatctacgaataatggtctgatccatgtctagcatcggacaacaacgcgagcaatgcgacgttccatcgccgacttgaacgtgtcctgcacgtgggtgcctgcagttgctcccattcctcctccggcgaattctcctcagtcatcatgatcggcacactactatcctggtatacagctgctgctatgcaagattgctaccccgaaccactataagaacctgctacgcccggtgtgaatgatgtgaaaaacagcagagcggccttcatctctatatgcctctcttctcttttctctccgcgacaccaacagtcatgcagcttatcttcacggttgcagccatcgcgagtgtggcagccgggtttcagtcggtgattagcgaaaagcaattttctcaggaatttctcgacaactacagcatcctgaagcactatggcggtaatggcccttactccagccgccggtcctacgggatctcgcgcgaacctcccgactcgtgctccgtcgaccaggtcatcatgatcatgcgtcatggcgagcgatatccgtctccagacctcggagcgagcatcgaagcagctctcgccaagatcaagtcctcgaacgtatccacataccagggcgacctggatttcctaaattcctggacctactatgtccccaatcactgtgcctacaatgccgagacctccaccggaccgtatgctggtctcctcgagggattcaagcgaggtagcgactaccgcgctcgttacggtcatttgtgggacggggagtcgattgtcccaatcttcgctgctggttaccagcgtatcattgctacttctcgaaaattcggcgagggtttcttcggcgccaactactccaccaatgcggccatcaatattatctcagaggcaaaggagatgggtgcaaatagcctcacacccacttgcgaccacgacaatgacaccagcacttgcaactccctgacaacggtgtggccacagttcaaagtcgctgcagcccgtttgaattctcagaaccccggtttggatctgaatgccactgacatctactatctgatgtgtatgtatagtctttcccatcctgcccctattgccgtatggacgacttactggttgcagccatggcttcctttgaattgaacgctcggccgtactccgactggatcaatgtttttacccttgatgagtgggtgacgtttggttacgttcaggacctgaattattattactgcgccgggtatgggtctctggttgtcccaggtctgggtcacagtcgctgatctttcacgcagcccaggagacaagaacatggccgctgtgggggccgtatatgtaaatgcatctctgactctcctcaaccaaggcccgtcagctggcacattatggtttaacttgttagtaccctcttcccagcaatggagcccctattcccggctgacccatgttgcagtgcccatgatacaaacatcacccccattcttgcggccctcggcgtcctcaccccggagcgtgatcttcccaccgaccgtgttgtcttcgatagcaagtggtcctccggggacatcgtcccccaggccggccacctgacaatcgagcgactgaactgcacgagcactgccgcatcacctgccggcgtctatgtccgcttggtccaaaatgaggccgtcatcccggttgaagcgtgccaatcaggtcctgggtactcttgctccctggccgacttcaccgaaattatgtccaagcagcttcccgatttcgtgtcgacttgcagtattcgctcgtcgtaccctcagtacctagacttctggtggaactacaatacgaccaccgatctgaactatccgaagggcccggttccctgtgccgagggagtggcgacaagctagtaggctctatgctgtcctcacggacatgtattccacaccactcctcccatcacaaccaggacgatgcttactgaacagtccgtttcatcatatgggggtgtctctgtagtatataatatgtcagtcttttccgcaatcttaggaatagaaggaattcaccgtcctggtccacagtcctctagtgggcaggaaaaatggcaatgctgacgtgtgtggaaaaataatatccagcggcgagcggccgctgggccgcccagcacctgtgtagcatcgctattctgcacctgccaagaactttttagtcacacgtagcttgatctactatatgaagtggagaacaaggctactttccctgggttgattggaaatgttgagatagtcacgagctgctatgaatgttttgttttgactacattaaagtacataggtgtacatggctgactgcttgctgctcctgggcaggatcttactgaagtaggtaaggaagagcaacatgaggccgtatgggcagataacctgaacgtgtgctacgtgagccccgacgggattatctttgatgatgcatgagtcttgccagggaatacaacatcataatacccagctccagacttgaaatccagtgttgaactcattatgcaggagttttgacattgtagttggccatgcaatctgtgtcagctaccctggcaatgcaacctctgaggtcaattcctatgccaccagtgcaatgggggcgacggctacctctgctgccccaataccaagcaatgtaattgcctgggaccaacgtgtactgcgggagatattattcaacccaaggtaatccaaggctttctctgtgggtttgggacctcaccgctgactgaaccaagaaaatgattactgccaggccatcgcaatgaaggagggaatcagccttgacgcagcaacttgtacttgaattatagctattgtactggcccgtcattccagattatagacgttccggggctaattcaagtcaatctctgggtgtgcagccggttggaaacattgagatgtatcctggctcgcgcctggcaggcgatgctccagtatcggcttgtgcggacccactgggtgttaccgtatccaaacgactgacgcttctggagtcatggcacggcactccatggactagggtggagacggctactcacttgggtcagggatcctctaga

SEQ ID NO: 14 - DNA, 2071 bp, Aspergillus niger
  exon (136)..(916)
  exon (971)..(1088)
  exon (1141)..(1245)
  exon (1305)..(1740)
gcatgctggaccgcaatctccgatcgccgggtataaaaggtcctccaaacccctctcggtcgatatgtaccccgctcgtcatctccaatcctctcgagagcaccttctccagcttttgtcaattgtaccttcgcaatgcctcgcacctctctcctcaccctggcctgtgctctggccacgggcgcatccgctttctcctacggcgctgccattcctcagtcaacccaggagaagcagttctctcaggagttccgcgatggctacagcatcctcaagcactacggtggtaacggaccctactccgagcgtgtgtcctacggtatcgctcgcgatcccccgaccagctgcgaggtcgatcaggtcatcatggtcaagcgtcacggagagcgctacccgtccccttcagccggcaaggacatcgaagaggccctggccaaggtctacagcatcaacactactgaatacaagggcgacctggccttcctgaacgactggacctactacgtccctaatgagtgctactacaacgccgagaccaccagcggcccctacgccggtttgctggacgcgtacaaccatggcaacgattacaaggctcgctacggccacctctggaacggtgagacggtcgtgcccttcttttctagtggctacggacgtgtcatcgagacggcccgcaagttcggtgagggtttctttggctacaactactccaccaacgctgccctcaacatcatctccgagtccgaggtcatgggcgcggacagcctcacgcccacctgtgacaccgacaacgaccagaccacctgcgacaacctgacttaccagctgccccagttcaaggtcgctgctgcccgcctaaactcccagaaccccggcatgaacctcaccgcatctgatgtctacaacctgatgggtatgtgattacggtacaatcattggctcaaacctccagctgacagcatcctagttatggcctcctttgagctcaatgctcgtcccttctccaactggatcaacgcctttacccaggacgaatgggtcagcttcggttacgttgaggatttgaactactactactgcgctgggtgagtttaccatttgatccattattgtcttggatcagctaacgatcgatagtcccggtgacaagaacatggctgctgtgggtgccgtctacgccaacgccagtctcaccctcctgaaccagggacccaaggaagccggctccttgttcttcaacttgtacgttctcggcagaatcagagtctcacaaaaagaaactcttcactaacatatagtagtgcccacgacaccaacatcacccccatcctcgccgccctaggcgtcctcatccccaacgaggaccttcctcttgaccgggtcgccttcggcaacccctactcgatcggcaacatcgtgcccatgggtggccatctgaccatcgagcgtctcagctgccaggccaccgccctctcggacgagggtacctacgtgcgtctggtgctgaacgaggctgtactccccttcaacgactgcacctccggaccgggctactcctgccctctggccaactacacctccatcctgaacaagaatctgccagactacacgaccacctgcaatgtctctgcgtcctacccgcagtatctgagcttctggtggaactacaacaccacgacggagctgaactaccgctctagccctattgcctgccaggagggtgatgctatggactagatgcagaggggtaggtcccgggatactttagtgatgattgatattcaagtttggtggtgacgatcaccttgttaatagtcttgtacagtcatacggtgaatgtaaataatgataatagcaatgatacatgttggaatctcgttttgttctttgtgtgcataggcgctttgggggtgtatttttaggcgttagacttattttcaattcgtgtataatgcggtcagtaaatgaatcatcaattattcaaatgcaatgctgtatacgtgaaactattgggttaagacgcagctactagctgactgcttggttactttctgtgtacaccgcatgc

SEQ ID NO: 15 - DNA, 1449 bp, Monascus purpureus
atgcctctcttctcttttctctccgcgacaccaacagtcatgcagcttatcttcacggttgcagccatcgcgagtgtggcagccgggtttcagtcggtgattagcgaaaagcaattttctcaggaatttctcgacaactacagcatcctgaagcactatggcggtaatggcccttactccagccgccggtcctacgggatctcgcgcgaacctcccgactcgtgctccgtcgaccaggtcatcatgatcatgcgtcatggcgagcgatatccgtctccagacctcggagcgagcatcgaagcagctctcgccaagatcaagtcctcgaacgtatccacataccagggcgacctggatttcctaaattcctggacctactatgtccccaatcactgtgcctacaatgccgagacctccaccggaccgtatgctggtctcctcgagggattcaagcgaggtagcgactaccgcgctcgttacggtcatttgtgggacggggagtcgattgtcccaatcttcgctgctggttaccagcgtatcattgctacttctcgaaaattcggcgagggtttcttcggcgccaactactccaccaatgcggccatcaatattatctcagaggcaaaggagatgggtgcaaatagcctcacacccacttgcgaccacgacaatgacaccagcacttgcaactccctgacaacggtgtggccacagttcaaagtcgctgcagcccgtttgaattctcagaaccccggtttggatctgaatgccactgacatctactatctgatgtccatggcttcctttgaattgaacgctcggccgtactccgactggatcaatgtttttacccttgatgagtgggtgacgtttggttacgttcaggacctgaattattattactgcgccggcccaggagacaagaacatggccgctgtgggggccgtatatgtaaatgcatctctgactctcctcaaccaaggcccgtcagctggcacattatggtttaactttgcccatgatacaaacatcacccccattcttgcggccctcggcgtcctcaccccggagcgtgatcttcccaccgaccgtgttgtcttcgatagcaagtggtcctccggggacatcgtcccccaggccggccacctgacaatcgagcgactgaactgcacgagcactgccgcatcacctgccggcgtctatgtccgcttggtccaaaatgaggccgtcatcccggttgaagcgtgccaatcaggtcctgggtactcttgctccctggccgacttcaccgaaattatgtccaagcagcttcccgatttcgtgtcgacttgcagtattcgctcgtcgtaccctcagtacctagacttctggtggaactacaatacgaccaccgatctgaactatccgaagggcccggttccctgtgccgagggagtggcgacaagctag

SEQ ID NO: 16 - protein, 482 aa, Monascus purpureus
MPLFSFLSATPTVMQLIFTVAAIASVAAGFQSVISEKQFSQEFLDNYSILKHYGGNGPYSSRRSYGISREPPDSCSVDQVIMIMRHGERYPSPDLGASIEAALAKIKSSNVSTYQGDLDFLNSWTYYVPNHCAYNAETSTGPYAGLLEGFKRGSDYRARYGHLWDGESIVPIFAAGYQRIIATSRKFGEGFFGANYSTNAAINIISEAKEMGANSLTPTCDHDNDTSTCNSLTTVWPQFKVAAARLNSQNPGLDLNATDIYYLMSMASFELNARPYSDWINVFTLDEWVTFGYVQDLNYYYCAGPGDKNMAAVGAVYVNASLTLLNQGPSAGTLWFNFAHDTNITPILAALGVLTPERDLPTDRVVFDSKWSSGDIVPQAGHLTIERLNCTSTAASPAGVYVRLVQNEAVIPVEACQSGPGYSCSLADFTEIMSKQLPDFVSTCSIRSSYPQYLDFWWNYNTTTDLNYPKGPVPCAEGVATS

SEQ ID NO: 17 - DNA, 1181 bp, Monascus purpureus
gatatcaatgagtacaactatatcaggctggttctgagacatgaatcgagcaatcagttgataatgcaactagtgtcgtgcagagcagacatgctcgaggaatgatgcaagtatggtctgataatcgagtgcaatcagcccagaagaatatctacatggactttttgagacgtatggaacatcaggtcatggccagtgccagataatcagacaatgacgagagggcaaagacatgaggagaggatgcattgactcttgagatagtagcatgatggggataacgttgtatggcttaattcatttctgacatctgatagtaatgccatgaatcatggaacaagaaaaatagcaccagaaagcacttcggtgtggcagatatgtagtgcagccggtgagtgctttgaggaatcccatcagcagacgcgatggaagcatctggaagcattatataagccattggtgcctgaggggaccagggcccgggccaactgttgttccgtgggtagatcaggtgacctgccatagccccttctagcggggataatcgtacattttatagttcattaggtatataccgtgttacatcaccactggggccgcgacgaagatccccttgtccctacggagcgacggaggacgttggcgggggattatcgcgtagagaatagcctaggcagagcgctgcagggaatacccgagaaatcaggcaagaagagcagcggcgatggatctatgatgcgtggcagtcacctgattggcgggcgtggagtctgcagcgcgcaaaatatcgtgattcttcctgctctgcccggtgtctgaaaccggaaaaccagctccaggcaggccattgccgcgtccgcggcacaactcggggcacggcagtgtcaaggacaacgaggcgaatcactggctgcgtttggccacccggtgtgtgcggtgtgcgccggtggaagacaaccagcagacgcgcgctgtttgatgacccctccgttgcgtcagtccattcctccccctccctgccccgccccgcgctgcctggcgacgtcccatactatattactcccgtcctcctctcttcctggctctctctttttcagattcatcaatccatcacatctcatcaacccatctcgatccatctcgatacatctcgatttcgtctaatatctatcactctatca

SEQ ID NO: 18 - DNA, 2456 bp, Monascus purpureus
  exon (1079)..(1129)
  intron (1130)..(1194)
  exon (1195)..(1208)
  intron (1209)..(1272)
  exon (1273)..(1324)
  intron (1325)..(1408)
  exon (1409)..(2090)
  intron (2091)..(2149)
  exon (2150)..(2346)
  CDS join(1079..1129, 1195..1208, 1273..1324, 1409..2090,2150..2346)
gatatcaatgagtacaactatatcaggctggttctgagacatgaatcgagcaatcagttgataatgcaactagtgacgtgcagagcagacatgctcgaggaatgatgcaagtatggtctgataatcgagtgcaatcagcccagaagaatatctacatggactttttgagacgtatggaacatcaggtcatggccagtgccagataatcagacaatgacgagagggcaaagacatgaggagaggatgcattgactcttgagatagtagcatgatggggataacgttgtatggcttaattcatttctgacatctgatagtaatgccatgaatcatggaacaagaaaaatagcaccagaaagcacttcggtgtggcagatatgtagtgcagccggtgagtgctttgagaatcccatcacgagacgcgatggaagcatctggaagcattatataagccattggtgcctgaggggaccaggcccgggccaactgttgttccgtgggtagatcaggtgacctgccatagccccttctagcggggataatcgtacattttatagttcattaggtatataccgtgttacatcaccactggggccgcgacgaagatccccttgtccctacggagcgacggaggacgttggcgggggattatcgcgtagagaatagcctaggcagagctgcagcggcaaaatatcgtgattcttcctgctttgcccggtgtctgaaaccggaaaaccagctccaggccattgcctccgcggcacaactcggggcacggcagtgtcaaggacaacgaggcgaatcactggctgcgtttggccacccggtgtgtgcggtgtgcgcggtggaagacaaccagcagagcgcgctgtttgatgacccctccgttgcgtcagtccattcctccccctccctgccccgccccggctgcctggcgacgtccatactatattactcccgtcctcctctcttcctggctctctctttttcagattcatcaatccatcacatctcatcaacccatctcgatccatctcgatacatctcgatttcgtctaatatctatcactctatcaaaatggttgtccccaaggttggaatcaacggcttcggtcgtatcggccgtattgtaagttccctctccctatcctcttgctcgtctcgtgccaataaatccctaacaatgtttccaaggtcttccgtaacgcgtaagtagctttctcgggcgttttccctggatccccagagagtatcctaacccttcgcctacagtatcgagcacgagggtgttgacatcgttgccgtcaacgaccccttcattgaggtccactatgctgtacgttccgttccattcctgcccagcatcgtcacctcgctcgagaagctaaccagacacgatatcgattaggcctacatgctcaagtatgacagcacccacggccgcttcaacggagccgtcgagttcgacggcaacacgctcatcgtcaacggcaagaagatcaagttctacgcagagagggaccccgctcagatcccctggagcgagactggccagtacgtcgttgagtccactggtgtcttcaccaagcaggagaaggcctcccttcacctgagagggtgtgccaagaaggtcatcatctccgctccctcttccgactcccccatgtttgtcatgggtgtcaacaacgaccaatacaccaaggacatcaccgtcctttccaacgcctcttgcaccaccaactgcttggctccccttgccaaggtcatcaatgacaagttcggcatcgtcgagggtctgatgaccacagtccactcctacactgctacccagaaggtcgtcgatggcccctccaacaaggactggagaggtggccgtaccgctgcccagaacatcatccccagctccaccggtgtgcctaaggcagtcggcaaggtcattccttccttgaacggcaagctcactggcatgtctatgcgtgtgcctacttccaacgcctccgttgtcgaccttactgcccgtctcgagaaggccgccacctacgacgagatcaagcaggccgtcaagaaggcctctgagcgccctctgaagggtgagtttaaaatgaccctcgatatgttgcacacggactcgattactgactaggactaggcatcctcggctacactgaggatgacgttgtctcctccgatctcaacggagacccccactcctccatcttcgatgccaaggctggtatcgccctcaactcgaacttcgtcaagctgttttcctggtacgacaacgagtggggttactcccgccgtgttatcgacctcattgcctatgcccaggtcgatgcccagtaattattagacgggctcctgagacgaaaagtctcctatgaaatcagaatgagcaatccctcaacgtactatccccacttcagctgaagtccctgcgcgaccggcagaggcct

SEQ ID NO: 19 - DNA, 21 bp, Artificial Sequence
  sense primer for amplification of Aspergillus oryzae amdS gene
aatcagtctgtagaatgctgg

SEQ ID NO: 20 - DNA, 21 bp, Artificial Sequence
  antisense primer for amplification of Aspergillus oryzae amdS gene
tctagatagtgcttatttgtc

SEQ ID NO: 21 - DNA, 29 bp, Artificial Sequence
  sense primer for amplification of Aspergillus nidulan salcB gene
gggaattcggcgtytgccayacagatctt

SEQ ID NO: 22 - DNA, 29 bp, Artificial Sequence
  antisense primer for amplification of Aspergillus nidulan salcB gene
ccgaattcgagkatgacrgcrtgcgcacc

SEQ ID NO: 23 - DNA, 26 bp, Artificial Sequence
  sense primer for amplification of Aspergillus niger aph gene
gggaattcatgcctcgcacctctctc

SEQ ID NO: 24 - DNA, 26 bp, Artificial Sequence
  antisense primer for amplification of Aspergillus niger aph gene
ccgaattcctagtccatagcatcacc

SEQ ID NO: 25 - DNA, 28 bp, Artificial Sequence
  sense primer for amplification of promoter region of Monascus purpureus GAPDH gene
ggtctagagatatcaatgagtacaacta

SEQ ID NO: 26 - DNA, 28 bp, Artificial Sequence
  antisense primer for amplification of promoter region of Monascus purpureus GAPDH gene
acaaccgaattctgatagagtgatagat

SEQ ID NO: 27 - DNA, 28 bp, Artificial Sequence
  sense primer for amplification of terminator region of Monascus purpureus GAPDH gene
caggaattcttagacgggctcctgagac

SEQ ID NO: 28 - DNA, 30 bp, Artificial Sequence
  antisense primer for amplification of terminator region of Monascus purpureus GAPDH gene
ccaagcttaggcctctgccggtcgcgcgca

SEQ ID NO: 29 - DNA, 27 bp, Artificial Sequence
  sense primer for amplification of promoter region of Monascus purpureus alcB gene
ggctcaggatccttcctcgtgagaagg

SEQ ID NO: 30 - DNA, 27 bp, Artificial Sequence
  antisense primer for amplification of promoter region of Monascus purpureus alcB gene
aggttcagccatgaattcaagagtgaa

SEQ ID NO: 31 - DNA, 30 bp, Artificial Sequence
  sense primer for amplification of terminator region of Monascus purpureus alcB gene
ctgaattcggttggatggttgacgagatgg

SEQ ID NO: 32 - DNA, 30 bp, Artificial Sequence
  antisense primer for amplification of terminator region of Monascus purpureus alcB gene
tcctcgagttctgcgcagaaacacccagct

SEQ ID NO: 33 - DNA, 27 bp, Artificial Sequence
  sense primer for amplification of promoter region of Monascus purpureus aph gene
ttagtcctgagctcatcattggaatgc

SEQ ID NO: 34 - DNA, 27 bp, Artificial Sequence
  antisense primer for amplification of promoter region of Monascus purpureus aph gene
gagaagagaggatcctagagatgaagg

SEQ ID NO: 35 - DNA, 27 bp, Artificial Sequence
  sense primer for amplification of terminator region of Monascus purpureus aph gene
acaagctagtaggatccatgctgtcct

SEQ ID NO: 36 - DNA, 27 bp, Artificial Sequence
  antisense primer for amplification of terminator region of Monascus purpureus aph gene
gttcttggcaggtaccgaatagcgatg

SEQ ID NO: 37 - DNA, 27 bp, Artificial Sequence
  sense primer for amplification of promoter region and signal sequence of Monascus purpureus aph gene
ttagtcctgagctcatcattggaatgc

SEQ ID NO: 38 - DNA, 48 bp, Artificial Sequence
  antisense primer for amplification of promoter region and signal sequence of Monascus purpureus aph gene
atcgcaagtggattgatttctcgacccggctgccacactcgcgatggc

SEQ ID NO: 39 - DNA, 49 bp, Artificial Sequence
  sense primer for amplification of DNA encoding mature polypeptide of Aspergillus niger phytase
gccatcgcgagtgtggcagccgggtcgagaaatcaatccacttgcgata

SEQ ID NO: 40 - DNA, 27 bp, Artificial Sequence
  antisense primer for amplification of DNA encoding mature polypeptide of Aspergillus niger phytase
gttcttggcaggtaccgaatagcgatg

SEQ ID NO: 41 - DNA, 28 bp, Artificial Sequence
  sense primer for amplification of DNA encoding signal peptide of Aspergillus oryzae Taka-amylase A
ggtctagagatatcaatgagtacaacta

SEQ ID NO: 42 - DNA, 48 bp, Artificial Sequence
  antisense primer for amplification of DNA encoding signal peptide of Aspergillus oryzae Taka-amylase A
atcgcaagtggattgatttctcgaagccaaagcaggtgccgcgacctg

SEQ ID NO: 43 - DNA, 48 bp, Artificial Sequence
  sense primer for amplification of DNA encoding mature polypeptide of Aspergillus niger phytase
caggtcgcggcacctgctttggcttcgagaaatcaatccacttgcgat

SEQ ID NO: 44 - DNA, 30 bp, Artificial Sequence; identical to SEQ ID NO: 28
  antisense primer for amplification of DNA encoding mature polypeptide of Aspergillus niger phytase